SEQUENCE LISTING (6 sequences)

SEQ ID NO: 1 - protein, 410 aa, Artificial Sequence
  Synthetic primer
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 2 - DNA, 28 bp, Artificial Sequence
  Synthetic primer
ttcttgagcaacaccctcttctgcagcc

SEQ ID NO: 3 - DNA, 32 bp, Artificial Sequence
  Synthetic primer
tcgccatataccggtcaaagaaatcttgtgcc

SEQ ID NO: 4 - DNA, 25 bp, Artificial Sequence
  Synthetic primer
tcacccacactgtgcccatctacga

SEQ ID NO: 5 - DNA, 22 bp, Artificial Sequence
  Synthetic primer
tgaggtagtcagtcaggtcccg

SEQ ID NO: 6 - protein, 6 aa, Artificial Sequence
  Synthetic peptide
RSRKRK

(19) United States
(12) Patent Application Publication
(10) Pub. No.: US 2012/0114636 A1
(43) Pub. Date: May 10, 2012

(54) LOW MOLECULAR WEIGHT CYCLIN E (LMW-E) AS A BIOMARKER FOR PERSONALIZATION OF CANCER THERAPIES

(75) Inventors: Khandan Keyomarsi, Houston, TX (US); Nikki Delk, Houston, TX (US); Kelly K. Hunt, Houston, TX (US); Hannah Wingate, Humble, TX (US); Elizabeth A. Mittendorf, Houston, TX (US)

(73) Assignees: The Board of Regents of the University of Texas System, Austin, TX (US); Research Development Foundation, Carson City, NV (US)

(21) Appl. No.: 13/255,380

(22) PCT Filed: Mar. 10, 2010
(86) PCT No.: PCT/US10/26781

§ 371 Date: Jan. 20, 2012

Related U.S. Application Data
(60) Provisional application No. 61/295,061, filed on Jan. 14, 2010.
(60) Provisional application No. 61/159,331, filed on Mar. 11, 2009.

(51) Int. Cl. A61K 39/395; A61P 35/00; A61K 31/566; A61K 31/553; G01N 33/577; A61K 31/4196

(52) U.S. Cl. 424/133.1; 435/7.23; 514/383; 514/177; 514/211.08

(57) ABSTRACT

Methods are disclosed for predicting a patient response to anti-Her2 therapy or anti-aromatase therapy. In certain embodiments, the methods involve the identification of low molecular weight cyclin E (LMW-E) in cancers, such as breast cancer cells, as a predictive and prognostic marker. In further embodiments, LMW-E expression by a cancerous or pre-cancerous cell may be used to predict response to an aromatase inhibitor and/or CDK2 inhibitor, and determination of LMW-E expression may be used in the personalization of cancer therapies.

This application claims priority to U.S. Application No. 61/159,331 filed on Mar. 11, 2009, and U.S. Application No. 61/295,061 file on Jan. 14, 2010, the entire disclosure of which are specifically incorporated herein by reference in its entirety without disclaimer.

This invention was made with government support under grants CA87458 and CA009599 awarded by the National Institutes of Health, and P50CA116199 awarded by the National Cancer Institute. The government has certain rights in the invention.

### BACKGROUND OF THE INVENTION

I. Field of the Invention

The present invention relates to methods and kits for treating cancer, especially breast cancer, and for identifying individuals likely to respond to a particular cancer treatment regimen. More specifically, the invention discloses the use of cyclin E and its low molecular weight isoforms (LMW-E), their expression and/or cytoplasmic accumulation in cancer cells, as a predictive and prognostic tool in cancer treatment.

II. Background and Description of Related Art

Cyclins are cell cycle regulatory proteins which have a central role in the control of cell proliferation in eukaryotic cells through their association with and activation of cyclin-dependent protein kinases (CDKs (Coates et al., 2007; Coombes et al., 2007; Forbes et al., 2008; Ellis and Ma, 2007; Thurlimann et al., 2005; Viale et al., 2008; Normanno et al., 2005)). Cyclins were first identified in marine invertebrates and have since been divided into several classes of cyclins, currently designated as cyclins A-H. Cyclins are distinguished on the basis of conserved sequence motifs, patterns of appearance and apparent functional roles during specific phases and regulatory points of the cell cycle in a variety of species.

The correlation between the deranged expression of cyclins and the loss of growth control in cancer is well established (Buckley, et al., 1993; Keyomarsi et al., 1993). The expression of cyclin E, in particular, is altered both qualitatively and quantitatively in many cancer cell types, being both overexpressed and present in low molecular weight isoforms (LMW-E). Full-length cyclin E (EL) regulates the cell cycle by promoting G1/S transition and inducing gene transcription, DNA replication, histone biosynthesis, and centrosome duplication in S phase (Moroy and Geisen, 2004). Overexpression of cyclin E is associated with advanced tumor stage and grade, poor response to therapy, and decreased survival (Dong et al., 2000; Keyomarsi et al., 2002; Lindahl et al., 2004; Melck et al., 2007; Potemski et al., 2006; Salani et al., 2007; Salon et al., 2007; Scuderi et al., 1996; Yasui et al., 1996). LMW-E isoforms have a more profound effect on cell cycle deregulation than full length cyclin E (EL) (Akli et al., 2004; Bedrosian et al., 2004; Corin et al., 2006; Porter et al., 2001; Wingate et al., 2003; Wingate et al., 2005) and are strongly correlated with aggressiveness and metastatic potential in breast and ovarian cancer (Davidson et al., 2007; Keyomarsi et al., 2002; Akli et al., 2007).

The variability of individual responses to chemotherapeutic regimens has long been a complication in effectively treating disease. Even within a relatively homogenous population, some patients respond well to a particular regimen, while other subjects respond poorly. This is especially difficult in the case of cancer treatment, which is generally expensive, inconveniently administered, and associated with numerous, often severe side-effects. The advent of molecularly-targeted drugs and personalized treatment regimens has intensified the need for reliable and sensitive methods of predicting individual responsiveness to chemotherapy. Such methods would allow physicians and other medical professionals to select, among potential treatments, those which a particular subject is likely to benefit from, thus reducing the subject's expense from and exposure to ineffective treatments and their side-effects.

### SUMMARY OF THE INVENTION

The present invention overcomes limitations in the prior art by providing methods of treating cancer, such as breast cancer, as well as predicting tumor aggressiveness and patient responsiveness to particular therapies. The overexpression and subcellular localization of cyclin E and its isoforms (LMW-E) are disclosed as indicators of tumor development and metastasis and as biomarkers of responsiveness to anti-Her2 single and combination therapies.

In addition, part of the invention is also based on the inventors' studies of anti-aromatase therapy including letrozole treatment. Although letrozole treatment of postmenopausal estrogen receptor-positive breast cancer reduces risk of early metastasis, resistance develops with time. The inventors contemplated that inhibition of cyclin E/CDK2 kinase activity through increased binding of the cell cycle inhibitor p27 to the complex may be a key mediator of the antiproliferative effects of letrozole. The inventors found that overexpression of LMW cyclin E can bypass this process and renders letrozole ineffective in mediating growth arrest and also that treatment of the cells with CDK2 inhibitor roscovitine overcomes the LMW cyclin E-mediated letrozole resistance. Lastly, the inventors showed that breast cancer patients whose tumors overexpress LMW cyclin E are more likely to recur after anti-aromatase treatment compared to those with low or no expression of LMW cyclin E. The inventors' data suggest responsiveness of CDK2 inhibitor therapy for cancer patients with LMW cyclin E expression, particularly postmenopausal women with ER-positive breast cancer.

Therefore, there may be provided a method of assessing a cancer patient's responsiveness to an anti-Her2 therapy or an anti-aromatase therapy, the method comprising determining LMW-E expression in cells of the patient's cancer for assessing a cancer patient's responsiveness to an anti-Her2 therapy or an anti-aromatase therapy, wherein: a) an overexpression of LMW-E in such cells relative to a normal control is an indication that the patient will more likely exhibit a favorable response to an anti-Her2 therapy; b) an overexpression of LMW-E in such cells relative to a normal control is an indication that the patient is less likely to exhibit a favorable response to an anti-aromatase therapy; and/or c) an expression level of LMW-E in such cells that is not overexpressed relative to a normal control is an indication that the patient is more likely to exhibit a favorable response to an anti-aromatase therapy.

In one embodiment for predicting response to an anti-Her2 therapy, the present method may be further defined as a method of assessing a cancer patient's responsiveness to anti-Her2 therapy, wherein an overexpression of LMW-E in such cells relative to a normal control is an indication that the patient will more likely exhibit a favorable response to anti-Her2 therapy. In particular, the cancer patient may have a Her-2 positive cancer.

In a further embodiment if the patient has or is determined to have the LMW-E overexpression, the method may further comprise treating the patient with an anti-Her2 therapy, an anti-CDK2 therapy, or a combination of an anti-Her2 therapy and an anti-CDK2 therapy.

In embodiments related to anti-aromatase therapy, the method may comprise such an assessment wherein a) an overexpression of LMW-E in such cells relative to a normal control is an indication that the patient is less likely to exhibit a favorable response to an anti-aromatase therapy; and/or b) an expression level of LMW-E in such cells that is not overexpressed relative to a normal control is an indication that the patient is more likely to exhibit a favorable response to an anti-aromatase therapy.

In certain embodiments, the method may further comprise treating the patient with an anti-CDK2 therapy if the patient has the LMW-E overexpression. In other embodiments, if the patient does not have the LMW-E overexpression, then the method may further comprise treating the patient with an anti-aromatase therapy. Non-limiting examples of the anti-aromatase therapy include letrozole, exemestane, or anastrozole.

The method may further comprise providing a report indicative of the results of such an assessment, wherein the report may be stored in a tangible medium. The report may be in a computer-readable format. For example, the report may be generated by a computer, a densitometer or an imaging device. In certain embodiments, the method may comprise using an immunologic reaction device, such as a photo-detection reader.

The assessment method of the present invention may also comprise determining the normal control level of LMW-E expression. In some aspects, the normal control level of LMW-E expression is the level of LMW-E in normal cells.

For determining the level of LMW-E expression, the method may further comprise subjecting the cells, or cellular material of such cells, to an assay that determines the level of LMW-E. In certain aspects, such an assay may comprise using an antibody that binds LMW-E, and determining the level of antibody binding.

Such an antibody may bind LMW-E only but not cyclin E. In other aspects, the assay may comprise using an antibody that binds both cyclin E and LMW-E, such as an antibody that recognize C-terminal of cyclin E. In such an embodiment, the assay may further comprise: a) separating cyclin E and LMW-E in the cells or cellular material, into essentially distinct preparations stratified by one or more of subcellular localization, molecular mass, net molecular charge, and topology; and b) measuring levels of antibody binding to LMW-E in the cells or the cellular material as compared to a normal control.

In certain embodiments, the method may comprise determining LMW-E expression by contacting the cells or cellular material thereof with a first antibody that binds cyclin E and does not bind LMW-E, and a second antibody that binds cyclin E and LMW-E. For example, the first antibody carries a first label and the second antibody carries a second label. The first and second label may be detected at the same time or in different times. In a certain aspect, the assay may be carried out in a device comprising a plurality of reaction chambers. The report for the assessment may be generated by a device that permits assessment of such an assay.

There may further be provided a method of treating a cancer patient having Her2-expressing cells, wherein said patient has been determined to have a higher expression of LMW-E in Her2-expressing cells of the patient's cancer relative to a normal control, the method comprising treating the patient with an anti-Her2 therapy, an anti-CDK2 therapy, or a combination of an anti-Her2 therapy and an anti-CDK2 therapy. For example, the anti-CDK2 therapy may be roscovitine. A particular example of the cancer is breast cancer.

In a further embodiment, there may be provided a method for treating a cancer patient whose cancer cells do not have LMW-E overexpression relative to a normal control, comprising treating such a patient with an anti-aromatase therapy, such as letrozole, exemestane, or anastrozole. There may also be provided a method for treating a cancer patient whose cancer cells have a higher LMW-E expression relative to a normal control or who has an unfavorable response to an anti-aromatase therapy, comprising treating the patient with an anti-CDK2 therapy, such as roscovitine. The cancer may be breast cancer, particularly the cells of the cancer express estrogen receptors (ER).

For assessing the aggressiveness and metastatic potential of a tumor, there may be provided a method comprising determining in cells of the tumor LMW-E cytoplasmic expression, and wherein higher LMW-E cytoplasmic accumulation in the cells of the tumor relative to a normal control is an indication that the tumor is aggressive and potentially metastatic. For example, the determination of LMW-E cytoplasmic accumulation in the cells of the tumor relative to a normal control is an indication that a patient bearing the tumor has a decreased chance of survival relative to a patient bearing a tumor without such a determination. In certain aspects, the normal control may be LMW-E cytoplasmic expression determined in a sample of normal cells or tissue.

The method may further comprise generating a report indicating the levels of LMW-E cytoplasmic accumulation, for example, the report may be generated by a mass spectrometer, an imaging or a scanning device.

Based on the inventors' discovery of the preferential cytoplasmic expression of LMW-E, the determination of LMW-E expression may be carried out by subjecting the cells, or the cellular material of such cells, to one or more assays that can detect the cytoplasmic expression of LMW-E. For example, the assay may comprise contacting the cells or cellular material with an antibody that binds cyclin E and LMW-E. In particular, the assay may comprise: a) separating cyclin E and LMW-E in the cells into essentially distinct preparations, stratified by subcellular localization, and optionally one or more of molecular mass, net molecular charge, and topology; and b) detecting LMW-E expression in cytoplasm of the cells. The separation of cyclin E and LMW-E may be carried out by cell fractionation, such as fractionation into cytoplasmic and non-cytoplasmic fractions or nuclear fractions. The separation may further comprise electrophoresis.

Non-limiting examples of the tumor or cancer may be selected from the group consisting of glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullar carcinoma, mastocytoma, mesothelioma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonic carcinoma, squamous cell carcinoma, basal cell carcinoma, fibrosarcoma, myxoma, myxosarcoma, liposarcoma, chondrosarcoma, osteogenic sarcoma, and leukemia. A particular example may be a Her-2 positive tumor or breast cancer.

As used herein, the term “cyclin E” refers to full length cyclin E unless otherwise specified.

As used herein, the terms “low molecular weight cyclin E isoform” or “LMW-E” refer to protein sequences which are at least 90% homologous to human cyclin E, and which have a molecular mass of less than about 50 kD. These terms refer to both LMW-E formed by proteolytic cleavage and LMW-E formed by alternative translation.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and/or advantages of the present invention will become apparent from the following detailed description. It should be understood that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: LMW isoforms are seen in tumor, but not normal, tissue extracts.

FIGS. 2A-2D: The LMW cyclin E isoforms have biologic characteristics that are unique from that of the full-length cyclin E.

FIGS. 3A-3C: The LMW isoforms of cyclin E prevent 76NE6 cells from arresting their cell cycle in G0/G1 phase upon removal of growth factors.

FIGS. 4A-4B: The LMW cyclin E isoforms generate genomic instability.

FIGS. 5A-5C: 76NE6 cells overexpressing the LMW-T1, but not EL, isoform of cyclin E form tumors in mice.

FIGS. 6A-6C: The LMW cyclin E isoforms bind more efficiently than the full-length cyclin E to CDK2.

FIGS. 7A-7C. Relationship between HER2 and cyclin E in breast cancer patients.

FIGS. 8A-8E. Effect of HER2 downregulation on cyclin E expression.

FIGS. 9A-9C. Effect of HER2 downregulation on cyclin E-associated kinase activity and cell cycle profiles.

FIGS. 10A-10D. Effect of decreased HER2-mediated cell signaling after treatment with trastuzumab.

FIGS. 11A-11B. Alteration in proliferation and cell cycle profiles after treatment with trastuzumab.

FIGS. 12A-12D. In vivo effects of trastuzumab therapy.

FIGS. 13A-13B. Cancer cells accumulate LMW-E in the cytoplasm and have cyclin E associated kinase activity in the cytoplasm.

FIGS. 14A-14C. Generation of functional cyclin E and Cdk2 protein complementation fusion proteins.

FIGS. 15A-15C. Protein complementation reveals LMW-E/Cdk2 complexes localize to the cytoplasm.

FIGS. 16A-16C. LMW-E are sensitive to proteasomal degradation.

FIGS. 17A-17B. Fbw7 reduces cyclin E-IFPN/Cdk2-IFPC complex formation.

FIGS. 18A-18B. Cytoplasmic cyclin E-IFPN/Cdk2-IFPC localization render the LMW-E isoforms less susceptible to Fbw7-mediated degradation.

FIGS. 19A-19B. Proposed model of the effect of subcellular localization on cyclin E stability and activity in normal versus tumor cells.

FIG. 20. Cytoplasmic cyclin E-IFPN/Cdk2-IFPC localization may render the LMW-E isoforms less susceptible to Fbw7-mediated degradation.

FIGS. 21A-21C. Relationship between ER status and cyclin E in breast cancer.

FIGS. 22A-22C. Effect of aromatase inhibitor treatment on proliferative response and cell cycle distribution of MCF-7/Ac1 cells.

FIGS. 23A-23D. In vitro antiproliferative effect of increasing concentrations of letrozole in the presence of 25 nM AD on MCF-7/Ac1 human breast cancer cells.

FIGS. 24A-24D. LMW but not full-length cyclin E overexpressing MCF-7/Ac1 cells could partially override the letrozole inhibition of AD-induced G1 exit and AD-induced CDK2 protein levels.

FIGS. 25A-25D. Roscovitine blocks the AD-induced increase in active (phosphorylated) CDK2 and LMW cyclin E overexpression cannot bypass this effect.

FIGS. 26A-26C. AI-treated patients with high LMW tumors have increased frequency of recurrence and increase levels of CDK2.

FIGS. 27A-27B. Synergistic effect of trastuzumab and roscovitine in breast cancer cell lines overexpressing HER2 and cyclin E. FIG. 27A. High throughput clonogenic assays were used to compare the cytotoxic effects of trastuzumab alone, roscovitine alone, and the combination of trastuzumab and roscovitine in SKBr3 and BT474 breast cancer cells (X axis: roscovitine μM; Y axis: trastuzumab μg/ml; Z axis: percentage non-viable cells). FIG. 27B. Isobologram analysis showed a synergistic interaction between the two agents. Isobologram analysis and graphs were obtained using CalcuSyn software, which performs drug dose—effect calculation using the median effect method. Experiments were performed in triplicate and representative data are shown.

FIG. 28. Effect of increasing concentrations of letrozole in the presence of 25 nM AD on CDK2 kinase activity, activated CDK2 and Rb phosphorylation of MCF7/Ac1 human breast cancer cells. MCF-7/Ac1 cells were treated with the indicated concentrations of letrozole and AD for 3 days. CDK2 kinase assays were performed by immunoprecipitating equal amounts of cell lysate (250 μg) with monoclonal antibodies to CDK2 (D12, sc-6248) coupled to protein G beads, using histone H1 as substrate. Letrozole blocks the AD-induced increase in activated CDK2 (phospho-T160) and phosphorylation of Rb. The same cell lysates were subjected to western blot analysis (50 μg of protein) with the indicated antibodies.

FIG. 29. LMW cyclin E levels achieved by adenoviral expression is comparable to the levels seen in human breast tumor samples. MCF7/Ac1 cells uninfected or infected with 1000 m.o.i LacZ or cyclin EL, T1 or T2 adenoviruses were subjected to western blot analysis (50 μg of protein) with cyclin E and Actin antibodies. Cyclin E levels were compared to the levels seen in 7 human breast tumor samples (P1 to P7) expressing high levels of LMW cyclin E.

FIG. 30. LMW Cyclin E (T1) overexpressing cells maintain a high CDK2 kinase activity that is insensitive to letrozole inhibition. MCF-7/Ac1 cells were cultured in IMEM with 10% charcoal-stripped serum medium (CSSM) without phenol red and with 600 μg/ml of G418 for 4 days before plating. Plates were then infected with LacZ (4000 m.o.i) or increasing concentration of cyclin E-T1 adenoviruses 24 hours before drug treatment. Cells were then left untreated (E2W, estrogen withdrawal) or treated with 25 nM AD (AD) or treated with 25 nM AD and 1 μM letrozole (AD+Let) and collected 3 days later for CDK2 kinase activity. CDK2 kinase assays were performed by immunoprecipitating equal amounts of cell lysate (250 μg) with monoclonal antibodies to CDK2 (D12, sc-6248) coupled to protein G beads, using histone H1 as substrate. The bar graph represents the densitometric values of the phosphorylated histone H1 substrate.

### DESCRIPTION OF ILLUSTRATIVE EMBODIMENTS

The present invention is based, in part, on the discovery by the inventors that the overexpression and cytoplasmic accumulation of low molecular weight forms of cyclin E (LMW-E) in the tumor cells of a patient, particularly Her-2 positive tumor cells can indicate the likelihood of response to anti-Her2 therapy by the patient. Certain aspects of the present invention also stem from the discovery of the tumorogenicity of LMW-E and the correlation between LMW-E cytoplasmic accumulation and cancer prognosis. Methods and kits provided herein may be useful in predicting patient responsiveness, identifying tumors that are predisposed to develop aggressively or to metastasize, and treating a patient having cancer, especially Her2+ cancer. In further embodiments, the invention is based on the discovery that anti-CDK2 therapy can overcome resistance of cyclin E-mediated resistance to aromatase inhibitors. Certain aspects of the invention are also based on the discovery that herceptin and CDK2 inhibitor roscovitine are synergistic in treating HER-2 positive cells that express LMW-E.

### I. Cyclin E and its Low Molecular Weight Isoforms

Cyclin E, complexed to its kinase partner, cyclin dependent kinase (CDK) 2, regulates G1 to S phase progression of the cell cycle (Lew et al., 1991; Dou et al., 1996). The cyclin E/CDK2 complex is normally negatively regulated by the cyclin dependent kinase inhibitors (CKIs) p21 and p27. The balance between G1 to S phase progression and the ability to halt the cell cycle is delicately controlled by cyclin/CDKs and CKIs in normal cells, but is often aberrant in cancer cells (Tannoch et al., 2000). In addition, normal cells, but not cancer cells, can exit the cell cycle before the restriction point if an environmental stressor is detected, such as a lack of nutrients. However, after the restriction point, cells are committed to replicate their DNA. Due to the crucial role that regulated expression and cyclin E activity plays in maintaining proliferative homeostasis, any defects in its expression could have a critical effect in tumorigenesis. Indeed, cyclin E expression is aberrant in many types of cancers including colorectal, gastric, ovarian, melanoma as well as breast cancer (Jong et al., 1999; Cam et al., 2001; Sui et al., 2001; So et al., 2000).

The form of cyclin E predominantly expressed in normal and tumor cells is the full-length 50 kDa isoform, considered wild-type, and referred to herein as EL. However, tumor cells are uniquely capable of post-translationally cleaving the full-length cyclin E (EL) into low molecular weight (LMW-E) isoforms. Elastase cleaves the EL into 44 kDa and 33 kDa isoforms (LMW-E), which are then phosphorylated. Clinical studies have indicated that cyclin E overexpression occurs in 25% of breast cancer tumors and is associated with poor prognosis (Keyomarsi et al., 2002) and the generation of LMW-E from EL is tumor specific (Harwell et al., 2000; Porter et al., 2001). In one study, high levels of total cyclin E (EL+LMW-E) or LMW-E were the most powerful discriminants of disease-free and overall survival, outperforming currently used clinical criteria including nodal status, stage, and estrogen-receptor status (Keyomarsi et al., 2002).

Accordingly, certain aspects of the present invention provide a method of identifying a tumor as being one or more of aggressive and potentially metastatic. The method comprises obtaining a sample of the tumor cells and identifying in the cells of the sample the presence of one or more of LMW-E overexpression, full-length cyclin E (EL) overexpression, and overexpression of EL plus LMW-E. In some embodiments, the expression of LMW-E and cyclin E in tumor cells is compared with the expression of LMW-E and cyclin E in a normal control sample, defined as a sample of normal or noncancerous cells. In other embodiments, the extent of overexpression of LMW-E or cyclin E or both in tumor cells relative to normal cells is an indication that the tumor may be aggressive or metastatic or both.

LMW isoforms of cyclin E, compared to full-length cyclin E, de-regulate the cell cycle and have unique biochemical properties. For example, the overexpression of LMW cyclin E in MCF-7 breast cancer cells results in resistance to the growth inhibiting effects of anti-estrogens (Akli et al., 2004), due to their resistance to inhibition by p21 and p27 (Wingate et al., 2005). N-terminal elastase cleavage of the 50 kDa, full length cyclin E protein between amino acids Q40-E45 or A69-D70, or alternative translation of cyclin E at M46, generates LMW-E proteins which lack the canonical nuclear localization sequence (NLS) and accumulate in the cytoplasm. Without wishing to be bound by theory, the loss of an NLS is most likely responsible for altered localization of LMW-E and may be important in the associated tumorogenicity, since the LMW-E can remain in the cytoplasm, interacting with CDK2 and promoting continuous cell cycle progression.

Accordingly, certain embodiments of the present invention provide a method of assessing the aggressiveness and/or the metastatic potential of a tumor. In these embodiments, a sample of the tumor cells may be obtained and analyzed for the presence of one or more of LMW-E overexpression and LMW-E cytoplasmic accumulation. In preferred embodiments, the identification of either LMW-E overexpression or LMW-E cytoplasmic accumulation or both in the cells of a tumor is an indication that the tumor is aggressive and potentially metastatic.

Certain embodiments of the present invention provide for assessment of cancer aggressiveness, metastatic potential, and lethality, as well as treatment of Her2+ tumors. Cancers which may be assessed and/or treated by a method described herein may include, but are not limited to, glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullar carcinoma, mastocytoma, mesothelioma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonic carcinoma, squamous cell carcinoma, base cell carcinoma, fibrosarcoma, myxoma, myxosarcoma, liposarcoma, chondrosarcoma, osteogenic sarcoma, leukemia, and the metastatic lesions secondary to these primary tumors.

In select embodiments, full length cyclin E (EL) is a protein having the amino acid sequence of SEQ ID NO:1 or having at least 90%, at least 95%, at least 96%, at least 97%, at least 98%, or at least 99%, at least 99.2%, or more amino acid sequence identity with SEQ ID NO:1.

The full length cyclin E protein (EL), a 50 kD protein, and the LMW-E, cyclin E isoforms having a molecular mass of less than 50 kD, and preferably between about 33 kD and about 45 kD, can be identified and measured in cells, or cellular material thereof, using essentially any appropriate protein expression identification method known in the art. In some embodiments, one or more labeled immunospecific antibodies or labeled binding partners for cyclin E and its isoforms may be used in immunoprecipitation, western blot analysis, FACS analysis, immunohistochemical analysis, ratiometric protein analysis, and fluorescence microscopy protocols to identify, quantitate and/or localize cyclin E and/or LMW-E expression. In other embodiments, cyclin E and/or LMW-E may be identified using mass spectrometry or competitive enzymatic analysis. In still other embodiments, the expression of cyclin E and its isoforms may be identified and quantified by analysis of messenger RNA encoding cyclin E. By way of a non-limiting example, cyclin E may be determined in cells or cellular material using the reverse transcriptase-polymerase chain reaction (RT-PCR), which is well known in the art.

In an aspect, an antibody or a labeled antibody may be useful in a method described herein as a means of discriminating between cyclin E and LMW-E. By way of nonlimiting example, an antibody which is immunospecific for an amino acid sequence that corresponds to all or part of N-terminal region of cyclin E, from between about the amino acid at position 1 to about the amino acid at position 50 of full length cyclin E (EL) may be useful in identifying and quantifying cyclin E, but not LMW-E. Alternatively, an antibody that is immunospecific for any part of the cyclin E sequence between about amino acid 50 to about the C-terminal amino acid of the protein may be used to identify both cyclin E and the LMW-E, and may be used to determine the expression level of cyclin E plus LMW-E, referred to herein as “total cyclin E”.

A method for determining the expression level of cyclin E and its isoforms as contemplated herein may employ a variety of appropriate labels including, but not limited to, radioactive elements, enzymes, chemicals that fluoresce when exposed to ultraviolet light, and fluorescent labels such as fluorescein, rhodamine, auramine, Texas Red, AMCA blue and Lucifer Yellow.

### II. Cyclin E Subcellular Localization for Prognosis

In a further aspect, a method for determining the expression of LMW-E may comprise cell fractionation, for example, any methods that separate cellular materials into a fraction that are substantially composed of cytoplasmic components (cytoplasmic fraction). Due to the cytoplasmic accumulation of LMW-E, an antibody or a fragment thereof which recognizes any part between about amino acid 50 to about the C-terminal amino acid of the full length cyclin E can be used to detect LMW-E cytoplasmic accumulation.

The inventors discovered that LMW-E show a marked subcellular localization to the cytoplasm and this separation is tumor-specific (Example 3). The inventors' findings suggest that the loss of the NH₂ terminus affects LMW-E subcellular localization, the localization of cyclin E-binding proteins, and LMW-E regulation by the proteasome. Thus, deregulated localization of cyclin E through the generation of LMW-E isoforms may be a mechanism by which cancer cells alter cyclin E regulation and function, imparting a growth advantage over normal cells. Indeed, tumor cells have higher cyclin E-associated kinase activity in the cytoplasm than do normal cells because tumor cells accumulate EL/Cdk2 and tumor-specific LMW-E/Cdk2 complexes in the cytoplasm. It is important to note that although preferentially accumulating in the cytoplasm, the inventors also found LMW-E isoforms and LMW-E/Cdk2 complexes in the nucleus of cancer cells. Previous studies have shown that the nuclear localization sequence motif of cyclin E is not required for cyclin E nuclear localization (Geisen and Moroy, 2002; Kelly et al., 1998; Porter et al., 2001). Furthermore, it is possible that LMW-Es are shuttled into the nucleus via protein-protein interactions.

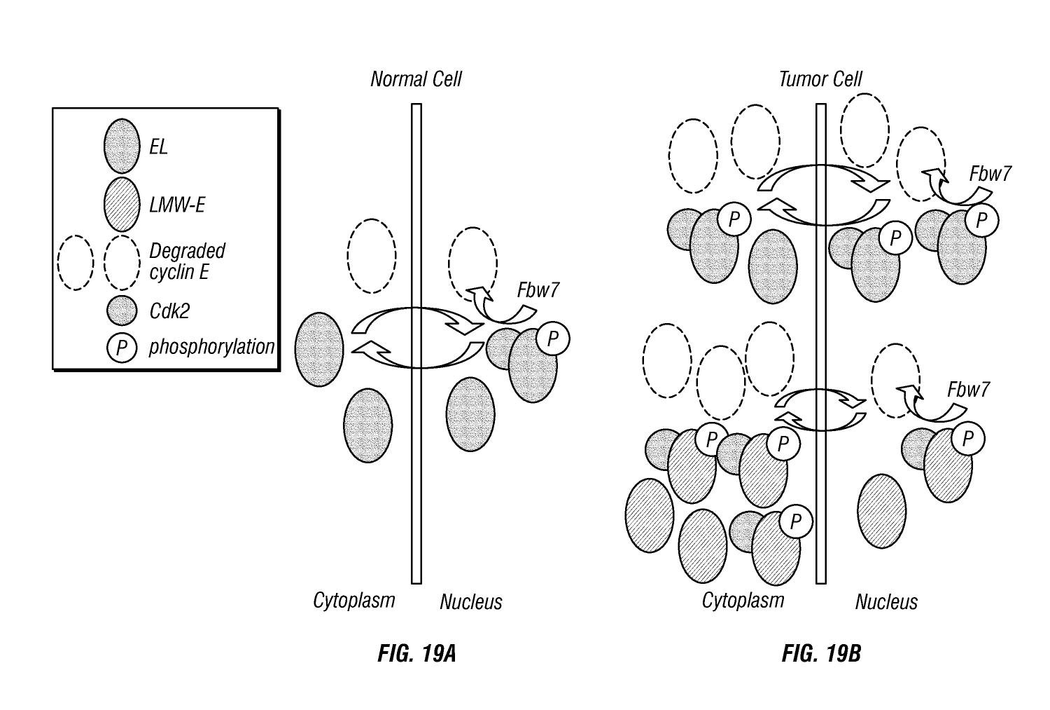

Cyclin E and cyclin E/Cdk2 subcellular localization is not a static process and, in fact, cyclin E/Cdk2 complexes are known to shuttle between the cytoplasm and nucleus (Jackman et al., 2002). Based on the inventors' results, they propose a model where, in tumor cells, EL and LMW-E proteins have differing nucleocytoplasmic shuttling dynamics that cause LMW-E/Cdk2 to be imported into the nucleus at a slower rate and/or exported more efficiently than EL/Cdk2 (FIGS. 19A-19B). The inventors also propose that these spatio-temporal differences affect EL and LMW-E proteasomal regulation in cancer cells, where the physiologically relevant, nuclear-localized Fbw7 isoforms target nuclear cyclin E/Cdk2 complexes (FIGS. 19A-19B).

### III. HER-2, CYCLIN E, and CDK2

An improved understanding of the pathways controlling cancer cell growth has led to refinements in the risk stratification of patients with cancer and the identification of targets for therapeutic agents. An example of this paradigm can be seen in breast cancer treatment, where finding a subset of breast tumors overexpressing HER2 (Her-2), the Human Epidermal growth factor Receptor 2 associated with breast cancer pathogenesis, has led to the development of trastuzumab, a monoclonal antibody directed against HER2, as adjuvant therapy in patients with HER2-overexpressing tumors (Slamon et al., 1987; Slamon et al., 1989). Unfortunately, this first agent developed for Her-2 blockade is effective as a single agent in only 35% of patients (Cobleigh et al., 1999; Vogel et al., 2002). Thus, HER2 overexpression alone does not ensure response to targeted therapy, suggesting a need for markers to identify those patients that will respond or that could serve as additional targets for novel therapeutics.

When overexpressed in cancer, particularly breast cancer, HER2 promotes enhanced growth and proliferation, and increases invasive and metastatic capabilities (Yarden and Sliwkowski, 2001; Yu and Hung, 2000). Clinical studies have shown that patients with HER2-overexpressing breast cancer have poorly differentiated tumors with high proliferative rates, positive lymph nodes, decreased hormone receptor expression (Slamon et al., 1987; Slamon et al., 1989; Gusterson et al., 1992; Wright et al., 1989), and an increased risk of recurrence and death due to breast cancer (Slamon et al., 1987; Slamon et al., 1989; Gusterson et al., 1992; Wright et al., 1989; McCann et al., 1991). The oncogenic effect of HER2/neu occurs through several mechanisms, including perturbation of the cell cycle. Specifically, the activation of the HER2/neu signal transduction pathway promotes cell proliferation by shortening the G1 phase of the cell cycle (Timms et al., 2002).

One cell cycle regulator with a crucial role in maintaining the G1/S transition is cyclin E (Ohtsubo and Roberts, 1993; Ohtsubo et al., 1995). Cyclin E is believed to be a factor in breast tumorigenesis secondary to its impact on cell cycle regulation; it is associated with a shorter G1 phase, a faster G1/S transition, increased cyclin E-associated kinase activity, and genomic instability (Akli et al., 2003; Akli et al., 2004; Dulic et al., 1993; Spruck et al., 1999). In a study of 395 patients with breast cancer, cyclin E was compared to other commonly used prognostic factors and found to be the most important predictor of death due to breast cancer (Keyomarsi et al., 2002).

The principal mode of cyclin E deregulation is at the protein level. Some breast cancer cell lines and human breast cancers express up to five low molecular weight (LMW) isoforms of cyclin E (Keyomarsi et al., 1997; Keyomarsi et al., 1994). These LMW forms are biologically relevant, as they activate CDK2 and phosphorylate substrates more efficiently than the full-length form. They are resistant to the growth inhibitory potential of the CDK inhibitors p21 and p27 and to anti-estrogens, and their overexpression induces genomic instability (Akli et al., 2004; Porter et al., 2001; Wingate et al., 2005). Recently, transgenic mice were generated with mammary-restricted expression of full-length or both full-length and LMW cyclin E. Mice overexpressing LMW cyclin E had a greater incidence of mammary tumors and distant metastasis than mice overexpressing full-length cyclin E, indicating that the LMW isoforms of cyclin E add metastatic potential to the overexpression of full-length cyclin E (Akli et al., 2007).

Without wishing to be bound by theory, the inventors contemplate that Her-2 signal transduction mediation and the G1 phase regulation of cyclin E may be interrelated such that a treatment strategy targeting both proteins may be better than targeting either one alone in patients whose tumors overexpress both Her-2 and LMW cyclin E. As shown in Example 5, the inventors discovered a synergistic effect of anti-Her2 therapy and anti-CDK2 therapy in breast cancer cells overexpressing Her2 and LMW-E.

In addition, a patient may be more effectively identified as potentially responsive to anti-Her2 therapy using the index of overexpression of both proteins rather than Her2 overexpression alone.

Accordingly, certain aspects of the present invention provide a method of identifying a patient as having an increased responsiveness to anti-Her2 therapy for cancer, wherein the method comprises obtaining a sample of cancerous or precancerous cells or tissue from the patient, and measuring in the sample the expression level of LMW-E and the expression level of Her-2. In some embodiments, a determination of both LMW-E overexpression and Her-2 overexpression is an indication that the patient has an increased responsiveness to anti-Her2 therapy. In other embodiments, the method additionally comprises obtaining a sample of noncancerous or normal cells, determining in the sample the expression level of LMW-E and the expression level of Her-2, and comparing the LMW-E and Her-2 expression levels in cancerous and noncancerous cells to identify the level of overexpression of each of LMW-E and Her-2.

### IV. Anti-aromatase Therapy

In certain aspects, the present invention provide methods for predicting responsiveness to anti-aromatase therapy and designing treatment plans in accordance with the predictions. For example, higher expression of LMW-E relative a normal control may serve as a marker for resistance to anti-aromatase therapy and may indicate a treatment plan using an anti-CDK2 therapy.

Endocrine therapy is an important part of the management of patients with hormone receptor positive breast cancer. Approximately 75 percent of postmenopausal women with breast cancer have tumors that express the estrogen receptor (ER) and/or progesterone receptor (PR) suggesting that they may benefit from such targeted therapy. These patients will routinely be offered a third generation aromatase inhibitor (AI) such as anastrozole, exemestane or letrozole. These agents have been demonstrated to be well tolerated and their use results in improved disease-free survival (DFS) compared to the selective estrogen receptor modulator, tamoxifen, when used in the adjuvant setting (Coates et al., 2007; Coombes et al., 2007; Forbes et al., 2008). Letrozole has also been shown to result in greater reduction in tumor size and increased utilization of breast conserving surgery when compared with tamoxifen in the neoadjuvant setting (Ellis and Ma, 2007).

Despite the effectiveness of AIs (aromatase inhibitors), not all patients respond to this treatment and in those who do, resistance develops after prolonged exposure. In a recent study, the value of proliferation as measured by Ki67, in predicting response to AIs was evaluated. This randomized, double blind, phase III study showed that letrozole improved disease-free survival compared to tamoxifen for postmenopausal women with hormone receptor-positive disease (Coates et al., 2007; Coombes et al., 2007; Forbes et al., 2008; Ellis and Ma, 2007; Thurlimann et al., 2005). The investigators found a greater benefit from letrozole compared to tamoxifen in tumors with a higher Ki67 labeling index, suggesting that high Ki67 labeling index levels may identify a patient group that could benefit from letrozole as their initial adjuvant therapy (Viale et al., 2008). With respect to resistance to AI therapy, in the majority of cases, ERα expression is not lost (Normanno et al., 2005), but there are alterations in downstream signaling genes and proteins. Increased growth factor signaling is also associated with resistance to endocrine therapy and suggests that inhibitors of signal transduction pathways could provide additional treatment options. The neoadjuvant setting provides the opportunity to identify genes that differ in expression with response (or lack thereof) to treatment. For example, in a recent neoadjuvant treatment study, increased expression of p44/p42 MAPK and HIF1a were independent predictors of resistance to letrozole (Generali et al., 2009). Taken together, these data suggest that identification and understanding of proteins that regulate response to AI treatment may provide critical information for the design of more effective treatment strategies.

The inventors discovered that a novel impact of dysregulation of cyclin E/CDK2 complex on the clinical benefit achieved from adjuvant endocrine therapy such as anti-aromatase therapy. In Example 4, the inventors showed that overexpression of the LMW forms of cyclin E render letrozole therapy ineffective in breast cancer cells which express both aromatase and estrogen receptor. The inventors contemplated that the mechanism of this effect is through LMW cyclin E-mediated induction of CDK2 activity. When LMW cyclin E is present, it results in higher CDK2 activity and resistance to p21 and p27 inhibition. Treatment of cells with letrozole leads to increased binding of p27 to CDK2 resulting in inactivation of CDK2. An event such as overexpression of LMW cyclin E, which can bypass this process will render letrozole ineffective in mediating a growth arrest in these cells. The inventors also showed that treatment of cells with roscovitine can overcome this LMW cyclin E-mediated letrozole resistance. As such, these data provide an alternative treatment option for those postmenopausal breast cancer patients whose tumors are ER positive, but express the LMW forms of cyclin E. The inventors showed that this subgroup of patients has a poor prognosis, with a median survival time of only 3.25 years. The inventors provided in vitro evidence, that if these patients were to be treated with letrozole, it is likely that they will not respond effectively to this treatment.

A major issue in the treatment of hormone receptor positive breast cancer is resistance to endocrine therapy. This resistance is intrinsic in up to 50% of patients and acquired in all patients with metastatic disease. Mechanisms of resistance to letrozole include a genetic polymorphism in the aromatase gene CYP19 (Colomer et al., 2008), high levels of ER expression driving transcription (Kuske et al., 2006) or a constitutively active estrogen receptor ER that does not require estrogen for activation (Masri et al., 2008). Cancer cells can also acquire resistance to letrozole by activation of the HER-2/MAPK pathway and in these cases trastuzumab plus letrozole has been shown to be more effective than either drug alone in letrozole-refractory tumors (Sabnis et al., 2009). Other growth factor pathways, including IGF receptor and the PI3K/AKT/mTOR pathways, have been demonstrated to play a role in resistance to endocrine agents and combination treatments targeting multiple pathways are more effective (Lisztwan et al., 2008; Beeram et al., 2007). Here the inventors show that activation of CDK2 by overexpression of the LMW forms of cyclin E is a novel mode of letrozole resistance; one that can be circumvented with CDK inhibitors.

Cyclin E protein is overexpressed and post-translationally cleaved by elastase into LMW isoforms (Porter et al., 2001). LMW cyclin E accumulation is tumor-specific and these isoforms have been found in multiple tumor types including breast, ovarian and colorectal cancers, and melanomas (Bales et al, 2005; Bedrosian et al., 2004; Corin et al., 2006; Davidson et al., 2007; Keyomarsi and Pardee, 1993). Furthermore, LMW cyclin E proteins have been shown to be strong correlative biomarkers in breast and ovarian cancers (Keyomarsi et al., 2002; Davidson et al., 2007). The LMW cyclin E isoforms have a more profound effect on cell cycle deregulation than the full-length cyclin E (EL) protein (Akli et al., 2004; Porter et al., 2001; Bedrosian et al., 2004; Corin et al., 2006; Wingate et al., 2003; Wingate et al., 2005) and transgenic mice expressing the LMW cyclin E isoforms have more mammary tumor development and metastasis than transgenic mice with the full-length cyclin E (EL) (Akli et al., 2007). Thus the LMW cyclin E isoforms appear more aggressive than EL in cell cycle abrogation and mammary tumor initiation and maintenance. Cyclin E has also been implicated in anti-estrogen resistance. A study found that the association between cyclin E and disease outcome was restricted to patients who were treated with tamoxifen in the adjuvant setting (Span et al., 2003). Another study using MCF-7 cells reported that overexpression of cyclin E could counteract tamoxifen-mediated growth arrest in human breast cancer patients (Dhillon and Mudryj, 2002). The laboratory has previously shown that overexpression of LMW cyclin E in breast cancer cells is associated with resistance to fulvestrant (Akli et al., 2004).

In certain aspects of the present invention the inventors discovered a novel mechanism of letrozole resistance through overexpression of LMW cyclin E leading to sustained activation of CDK2. In certain aspects, there may be provided a method for prediciting that patients with high LMW cyclin E levels (and possibly ER positive) tumors would likely not respond to letrozole treatment but could benefit from a therapy targeting the cyclin E/CDK2 complexes such as roscovitine (Seliciclib or CYC202).

Until now, the use of CDK inhibitors in human malignancies has been of limited success. This may be due to suboptimal selection of the group of patients that would benefit the most from the therapy. The inventors show in a model system that the conversion of androstenedione into estrogen by the aromatase enzyme activity strongly stimulates the growth of breast cancer cells by increasing the CDK2 kinase activity leading to increase in the S-phase fraction. The inventors' study shows that letrozole treatment blocks the AD-induced increase in S-phase fraction which would be translated to a low Ki67 labeling index in a responding tumor. The Ki67 labeling index before and after neoadjuvant endocrine therapy could identify the non-responding ER-positive, LMW cyclin E positive tumors that could benefit from a CDK2 targeted therapy. Additionally, certain aspects of the invention provide methods to identify the population of patients that may benefit from CDK inhibitors (i.e. overcoming the weaknesses of prior studies that were limited by poor patient selection) and that the data suggest that tumors from patients with ER positive disease could be assessed for expression of LMW cyclin E in an effort to predict who may respond to anti-aromatase therapy such as letrozole and who could also benefit from CDK2 targeted therapy.

Aromatase is an enzyme that synthesizes estrogen. Anti-aromatase therapy may include any therapy targeting aromatase known in the art, include siRNA, antisensense nucleotides, small molecule inhibitors, inhibitory antibodies or fragments thereof, and the like. Anti-aromatase therapy block the synthesis of estrogen. This lowers the estrogen level, and slows the growth of cancers.

Aromatase inhibitors (AI) are a class of drugs that may be used in the treatment of breast cancer and ovarian cancer in postmenopausal women. Aromatase inhibitors work by inhibiting the action of the enzyme aromatase, which converts androgens into estrogens by a process called aromatization. AIs could be categorized into two types: irreversible steroidal inhibitors such as exemestane form a permanent bond with the aromatase enzyme complex; non-steroidal inhibitors (such as anastrozole, letrozole) inhibit the enzyme by reversible competition. Some of the aromatase inhibitors in use include:

Non-selective: Aminoglutethimide, Testolactone (Teslac)

Selective: Anastrozole (Arimidex), Letrozole (Femara), Exemestane (Aromasin), Vorozole (Rivizor), Formestane (Lentaron), Fadrozole (Afema),

Unknown/ungrouped: 4-androstene-3,6,17-trione (“6-OXO”, marketed as a nutritional supplement for athletes and weight lifters), 1,4,6-androstatrien-3,17-dione (ATD), 4-hydroxyandrostenedione.

### V. Combination Therapy

In an aspect, the present invention provides a method of treating patient having a Her2+ cancer, wherein the method comprises identifying a patient having increased responsiveness to anti-Her2 therapy and administering to the patient a therapeutically effective amount of an anti-Her2 agent and a CDK-2 inhibitor. In another aspect, the method may comprise administering to the patient a therapeutically effective amount of an anti-Her2 agent and an elastase inhibitor.

In certain embodiments, a patient is identified as a candidate for anti-Her2 therapy using a method described herein. In other embodiments, a patient is identified as having increased responsiveness to anti-Her2 therapy by obtaining a sample of cancerous or precancerous cells or tissue from the patient, and measuring in the sample one or more of LMW-E overexpression, cyclin E overexpression, and LMW-E cytoplasmic accumulation. In preferred embodiments, a patient having a Her2+ cancer, in particular a Her2+ breast cancer, and LMW-E overexpression or LMW-E cytoplasmic accumulation is administered a therapeutically effective amount of an anti-Her2 agent and one or more of a CDK-2 inhibitor and an elastase inhibitor.

As referred to herein, a Her2+ cancer is a cancer in which cells overexpress Her2 relative to a normal control. Also as used herein, an “anti-Her2 agent” is an agent which inhibits, abrogates or blocks signal transduction activity by Her2.

Essentially any anti-Her2 agent known in the art may be useful for a method described herein. By way of non-limiting example, the most well known inhibitor of Her2/neu signaling currently is trastuzumab.

Essentially any inhibitor of elastase or of CDK-2 known in the art may be useful for a method described herein, for example, elastase inhibitors, elafin and sivelestat, and CDK-2 inhibitors such as roscovotine, olomoucine, flavopiridol, staurosporine, purine analogs or derivatives thereof, pyrimidine analogs or derivatives thereof, oxindoles, diarylureas, paullones, purvalanols, butyrolactone, and aloisine A.

The terms “subject” and “patient” may be used interchangeably herein, and can include all mammals, especially humans.

Administration of an “effective amount” of an anti-Her2 agent and one or more of a CDK-2 inhibitor and an elastase inhibitor is defined as an amount effective, at dosages and for periods of time necessary to achieve the desired result. The effective amount of an anti-Her2 agent, a CDK-2 inhibitor, and an elastase inhibitor may vary according to factors such as the disease state, age, sex, and weight of the subject. Using methods well known in the clinical arts, dosage regimen may be adjusted to provide the optimal therapeutic response. For example, several divided doses may be administered daily or the dose may be proportionally reduced as indicated by the exigencies of the therapeutic situation.

An anti-Her2 agent, a CDK-2 inhibitor, and an elastase inhibitor used in accordance with a method of the invention can be an aerosolized powder or liquid, a liquid, a solid or a semisolid and can be formulated in, for example, pills, tablets, creams, ointments, inhalants, gelatin capsules, capsules, suppositories, soft gelatin capsules, gels, membranes, tubelets, solutions or suspensions.

An anti-Her2 agent, a CDK-2 inhibitor, and an elastase inhibitor of the present invention may comprise different types of carriers depending on whether it is to be administered in solid, liquid or aerosol form, and whether it need to be sterile for such routes of administration as injection. A pharmaceutical composition of the invention can be intended for administration to humans or other animals. Dosages to be administered depend on individual needs, on the desired effect and on the chosen route of administration.

In accordance with the present invention, any of an anti-Her2 agent, a CDK-2 inhibitor, or an elastase inhibitor can be administered intravenously, intradermally, intraarterially, intraperitoneally, intralesionally, intracranially, intraarticularly, intraprostaticaly, intrapleurally, intrasynovially, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularly, orally, topically, by inhalation, infusion, continuous infusion, localized perfusion, via a catheter, via a lavage, in lipid compositions (e.g., liposomes), or by other method or any combination of the forgoing as would be known to one of ordinary skill in the art.

A pharmaceutical composition can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (2005). The phrase “pharmaceutical or pharmacologically acceptable” refers to molecular entities and compositions that do not produce an adverse, allergic or other untoward reaction when administered to a subject, such as, for example, a human, as appropriate.

In an aspect, a pharmaceutical composition of the present invention can comprise a therapeutically effective amount of an anti-Her2 agent and one or more of a CDK-2 inhibitor and an elastase inhibitor. The phrase “therapeutically effective amount” refers to an amount of a composition required to achieve a desired medical result, in particular, to achieve the treatment a Her2+ cancer. The preparation of therapeutically effective compositions will be known to those of skill in the art in light of the present disclosure, as exemplified by Remington's Pharmaceutical Sciences (2005), incorporated herein by reference. Moreover, for animal (e.g., human) administration, it will be understood that preparations should meet sterility, pyrogenicity, general safety and purity standards as required by FDA Office of Biological Standards.

As used herein, the phrase “a therapeutically effective amount” refers not only to active ingredients, but also includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with active agent described herein, its use in the present compositions is contemplated.

The actual required amount of a composition of the present invention administered to a patient can be determined by physical and physiological factors such as body weight, severity of condition, the type of disease being treated, previous or concurrent therapeutic interventions, idiopathy of the patient and on the route of administration. The practitioner of ordinary skill will rely on methods well established in the art to determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, carbohydrates, sodium chloride or combinations thereof.

Sterile injectable solutions may prepared by incorporating anti-Her2 agent and/or an elastase inhibitor in the required amount of the appropriate solvent with various amounts of the other ingredients enumerated above, as required, followed by filtered sterilization. Generally, dispersions are prepared by incorporating the various sterilized active ingredients into a sterile vehicle which contains the basic dispersion medium and/or the other ingredients.

In the case of sterile powders for the preparation of sterile injectable solutions, suspensions or emulsion, the preferred methods of preparation are vacuum-drying or freeze-drying or lyophilization techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

In some embodiments, an anti-Her2 agent, a CDK-2 inhibitor, or an elastase inhibitor disclosed herein may be administered to the airways of a subject by any suitable means. In particular, one or more of an anti-Her2 agent, a CDK-2 inhibitor, and an elastase inhibitor can be administered by generating an aerosol comprised of respirable particles, the respirable particles comprised of one or more of an anti-Her2 agent, a CDK-2 inhibitor, and an elastase inhibitor, which particles the subject inhales. The respirable particles may be liquid or solid. The particles may optionally contain other therapeutic ingredients.

In an aspect, the present invention encompasses combination therapy for Her2+ cancer wherein an anti-Her2 agent, and a CDK-2 inhibitor are comprised in a pharmaceutical composition with one or more pharmaceutically acceptable excipients. In this aspect, a pharmaceutical composition comprising an anti-Her2 agent and one or more of a CDK-2 inhibitor and an elastase inhibitor can be used in accordance with a method disclosed herein.

In particular embodiments, prolonged absorption of an injectable composition can be brought about by the use in the compositions of agents delaying absorption, such as, for example, aluminum monostearate, gelatin or combinations thereof.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein

### EXAMPLES

The following examples are included to demonstrate preferred embodiments of the invention. It should be appreciated by those of skill in the art that the techniques disclosed in the following example represent techniques identified by the applicant to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Low Molecular Weight Cyclin E is Tumor Specific and Tumorigenic

Here is presented the demonstration of LMW-E tumorogenicity and studies which provide support for the routine assessment of tumor biopsy samples for LMW cyclin E expression as a prognostic and potentially predictive marker in breast cancer patients.

### Materials and Methods

Human tissue samples: Tumor and adjacent normal tissue were obtained at the time of surgical intervention from 340 patients with stage I or II breast cancer. Patients signed a study-specific informed consent form for participation in this protocol, which was approved by the institutional review board at The University of Texas M. D. Anderson Cancer Center. Following surgical resection of the primary tumor and regional lymph nodes, specimens were examined in pathology, and fresh tumor and normal tissue were collected by the pathologist and were processed to obtain protein lysates. Protein lysates were subjected to western blotting followed by densitometric analysis with use of ImageQuant Total Lab software (Amersham Biosciences, Piscataway, N.J.). Each band was quantified and the LMW cyclin E bands were added together. Levels of LMW cyclin E and full-length cyclin E in tumor specimens were normalized against the levels of full-length cyclin E in adjacent normal tissue.

Western blot and kinase analysis: Cell lysates were prepared and subjected to western blot analysis as previously described (Rao et al., 1998). Monoclonal antibodies to the c-terminus of cyclin E (HE-12, Santa Cruz Biotechnology) or actin (Roche Molecular Biochemicals, Indianapolis, Ind.) were used at a concentration of 0.11 μg/mL for 1.5 hours. Immunoprecipitations were carried out using 300 μg of cell lysates with a polyclonal antibody to cyclin E using histone H1 (Roche Molecular Biochemicals) as a substrate as previously described (Chen et al., 1999).

Biacore studies: Cyclin E binding to CDK2 was monitored in real time with a Biacore 2000 instrument (Biacore AB, Uppsala, Sweden). CDK2 was purified from sf-9 cells using a protocol provided by Dr. Laurent Meijer (CNRS, Roscoff, France). The resultant CDK2 was tested for purity on a silver stained sodium dodecyl sulfatepolyacrylamide gel and tested for activity using a kinase assay. The freshly prepared CDK2 was immobilized on a CM5 chip (Biacore AB) using amine coupling

Surface preparation: The CM5 chips were normalized with 40% BlAnormalizing solution (Biacore AB). Two of the channels on each chip were then coated with the ligand, CDK2. This was achieved by injecting CDK2 diluted 1:1 with 0.1M sodium acetate, pH=3.5, at a flow rate of SA/minute for 50 minutes in HBS-P buffer (0.01 M HEPES pH 7.4, 0.15M NaCl, 0.005% v/v tween20) over the activated surface. Bovine Serum Albumin (BSA) was coupled to one of the flow channels of each chip, to be used as a non-specific binding control: This was accomplished by injecting 50 nM BSA over the flow channel using the same conditions as for the CDK2. One of the flow channels was left blank to serve as a background control. Finally, the uncoupled groups on the surface were deactivated by injecting 1 M ethanolamine-HCl, pH 8.5 over all of the channels.

Interaction analysis: Cyclin E samples were prepared from sf-9 cell lysates overexpressing each of the LMW cyclin E isoforms (EL, T1 or T2). Samples were diluted to 1 μg/μL, 500 ng/μL, 250 ng/μL and 125 ng/μL concentrations using sterile water. 70 μL of analyte was injected over all four channels of the chips at a flow rate of 30 μL/minute in 50 mM Tris pH 7.5, 10 mM MgCl₂, 0.005% v/v Tween20. Each analyte was injected twice in increasing concentrations, with water injections between each concentration for normalization.

Regeneration: After each injection, the surface was regenerated with 5 μL., of HBS-EP buffer (0.01M HEPES pH 7.4, 0.15M NaCl, 3 mM EDTA, 0.005% v/v surfactant P20).

Sample Recovery: In the experiments in which bound protein was collected for further tests, the flow rate was set to 30 μL/minute. One hundred microliters of each cyclin E sample was co-injected with 60 μL of HBS-EP buffer and 100 μL of the eluted sample was recovered and subjected to western blotting. Data analysis: Scrubber2 software (Center for Biomolecular Interaction Analysis, University of Utah, (available at world wide web via.cores.utah.edu/interaction/scrubber.html) was used for all binding analyses.

Cell culture conditions: The 76NE6 cell line was maintained in DFCI-1 media as described (Band and Sager, 1989). Transfections were carried out with FuGene Transfection Reagent (Roche Molecular Biochemicals, Indianapolis, Ind.) according to the manufacturer's instructions. pcDNA 4.0 plasmids (Invitrogen, Carlsbad, Calif.) containing either the cyclin EL-Flag, T1-Flag or no insert and the gene for Zeocin resistance were linearized for transfection. Stable pools were generated by selecting for the cells containing the vector using 2.0 ng/mL of Zeocin for 16 days and then maintaining the cells in 10 μg/mL of Zeocin. Stable clones were selected from the pools that were plated at 3% confluency until colonies formed. The colonies were isolated using cloning rings. The stable clones were then maintained in 10 μg/mL of Zeocin.

Clonogenic assays: A total of 100, 500 or 1000 cells were plated in 100 mm tissue culture dishes. The plates were incubated for 14 days and then stained with 0.05% crystal violet.

Cell synchronizations: 1×10⁶ cells of each 76NE6 clonal cell line: empty vector (4.0), full-length cyclin E (EL) and LMW cyclin E (T1) were plated in ten 100 mm plates for cell cycle analysis and 3×10⁵ cells of each clone were plated in duplicate wells of ten 6-well plates for Ki-67 staining experiments in complete DFCI-1 medium (Band and Sager, 1989). Twenty four hours after plating the cells, the media was changed to DFCI-3 (Band and Sager, 1989) to deplete the cells of growth factors. To characterize the cells during synchronization, they were harvested at the 0 hour time point. A 100 mm plate and the duplicate wells of the E-well plates were harvested for flow cytometric analysis or Ki-67 staining every 12 hours thereafter for 120 hours, as previously described (McGahren-Murray et al., 2006). To characterize the synchronous cells, the DFCI-3 media was changed to DFCI-1 media after 72 hrs and this was the 0 hr time point. Cells were collected every 3 hours for 33 hours thereafter for flow cytometric analysis and western blotting.

Ki-67 staining: 3×10⁵ cells of each clone (empty vector, EL and T1) were plated in duplicate wells of 6-well plates on top of cover-slips that were sterilely placed in each well. The cells were synchronized as described above. At each time point, cover slips were washed with cold phosphate-buffered saline (PBS) and fixed for 1 minute by adding 500 μL of cold fixative (1:1 v/v mix of methanol and acetone). After 3 more washes, cells were incubated with 50 μL of fluorescein isothyocyanate (FITC) conjugated Ki-67 antibody (Abcam, Cambridge, Mass.), diluted 1:10 with PBS for 2 hours, then washed again. ProLong Gold anti-fade reagent with 4′,6-Diamidino-2-phenylindole (DAPI, Invitrogen) was dropped onto microscope slides, which were then sealed with clear nail polish. Slides were analyzed within 24 hours using the 20× objective of a Nikon Optiphot microscope with an attached digital camera. The percentage of Ki-67 positive cells was determined by taking the ratio of Ki-67 positive cells to the number of DAPI positive cells.

Metaphase spread analysis: 1.5×10⁶ cells of each 76NE6 clone (empty vector, EL and T1) were plated on 100 mm plates. Fresh medium was added to the cells at 6 and 18 hours until they reached −80% confluency, at which point 20 ng/mL Colcemid was added to each plate for 4 hours. Cells were microscopically observed for the presence of metaphase cells (enlarged, translucent cells that can come off the plate with gentle tapping). The metaphase spreads were performed and analyzed for genetic instability by the Molecular Cytogenetics Facility at M. D. Anderson Cancer Center. Statistical differences were determined using the student t test with a 95% confidence interval. P values less than 0.05 were considered statistically significant.

In vivo tumorigenicity assays: Nude mice were purchased from Charles River Laboratories (Wilmington, Mass.) and maintained in the Department of Veterinary Medicine at the M. D. Anderson Cancer Center according to approved protocols. The mice were irradiated with 3.5 Gy of gamma-radiation from a ¹³⁷Cs source. At 24 hours after irradiation, 10 mice were injected with each of the following cell lines: MDA-MB-468 (as a positive control), 76NE6-empty vector clone, as a negative control, 76NE6-EL clone and 76NE6-T1. 72 hours after irradiation, 10 additional mice were injected with 76NE6-T1. Of the 10 mice, 5 were injected with 1×10⁷ cells suspended in 100 μL media (DFCI-1) and the other 5 were injected with 1×10⁷ cells suspended in 100 μL of a 1:4 Matrigel (Becton Dickinson and Company, Franklin Lakes, N.J.): Media mix. The cell suspensions were injected subcutaneously into the mammary fat pad of the mice using an 18-gauge needle. The mice were weighed and the diameter of their tumors was measured using calipers 3 times a week. Mice were sacrificed using CO₂ inhalation when tumors reached 12 mm in diameter. The tumors were harvested for histological analysis or for expansion in tissue culture for re-injection into mice. Tumors submitted for histopathologic analysis were fixed in 10% neutral buffered formalin, routinely processed, paraffin embedded, and serially sectioned at a nominal 5 μm.

Staining of tumor sections: Serial sections were stained with hematoxylin and eosin and cyclin E was identified with a polyclonal anti-Cyclin E antibody (Santa Cruz Biotechnology) Immunostaining was done by using Vectors ABC kit and the BCIP/NBT Chromagen detection system (Vector Laboratories, Burlingame, Calif.).

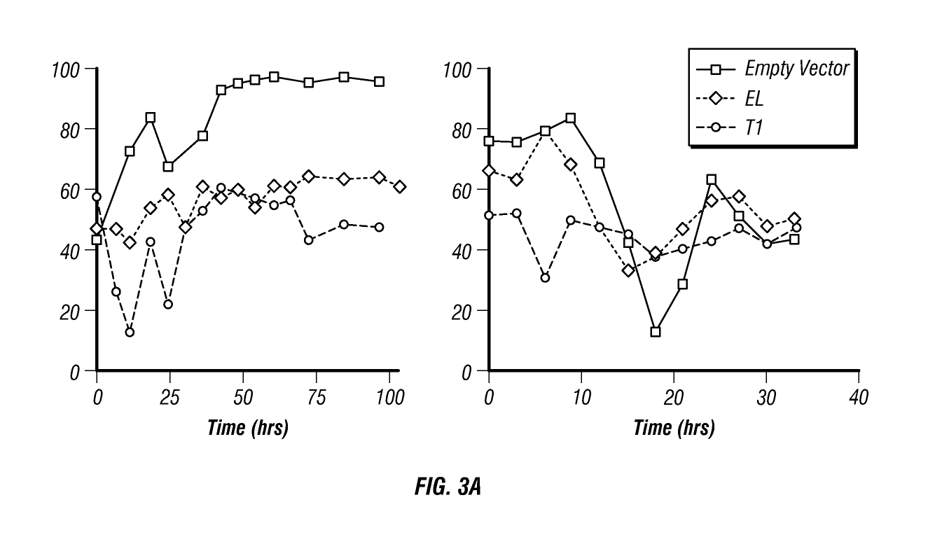

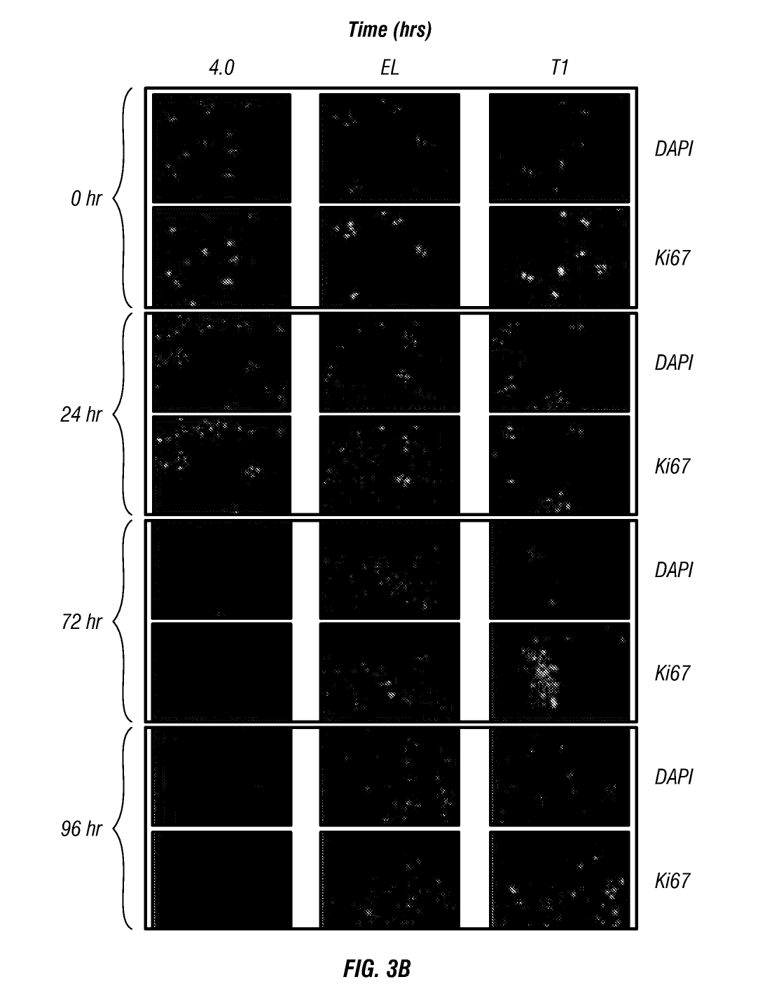

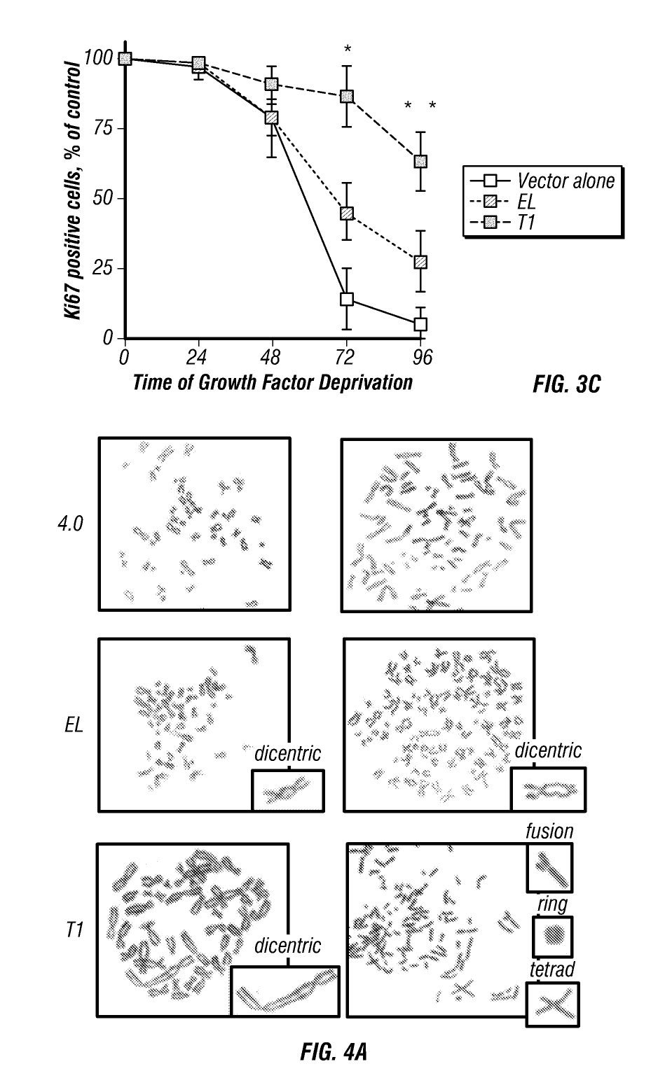

Statistical Analysis: All values for LMW cyclin E in the tumor and normal samples, and all values for full-length cyclin E in the tumor samples were normalized to the full-length cyclin E values in the normal tissue samples. For statistical analyses to determine correlation, the sign test was used, comparing the ratio of paired samples' deviation from 1. For determination of p values for Ki67 data presented in FIGS. 3A-3C, for each time point, the Kruskal-Wallis test was used to compare the fractions of proliferating cells for the 3 conditions (4, EL, T1). To compare EL to T1 Mann-Whitney test was then used. All other p values were calculated using a 2-sided student's t test. For bar graphs, error bars indicate 95% confidence intervals.

### Results:

The LMW isoforms of cyclin E are tumor specific. The LMW isoforms of cyclin E have been associated with an aggressive phenotype in several types of cancer including breast, ovarian, gastric and colorectal cancers as well as melanoma (Gusterson et al., 1992; Bales et al., 2005; Bedrosian et al., 2004; Milne et al., 2008; Corin et al., 2006). To determine whether the LMW isoforms are specific to tumor tissue, breast cancer tissue along with adjacent non-tumor tissue samples were prospectively collected from 340 women with stage I or II breast cancer at the M. D. Anderson Cancer Center.

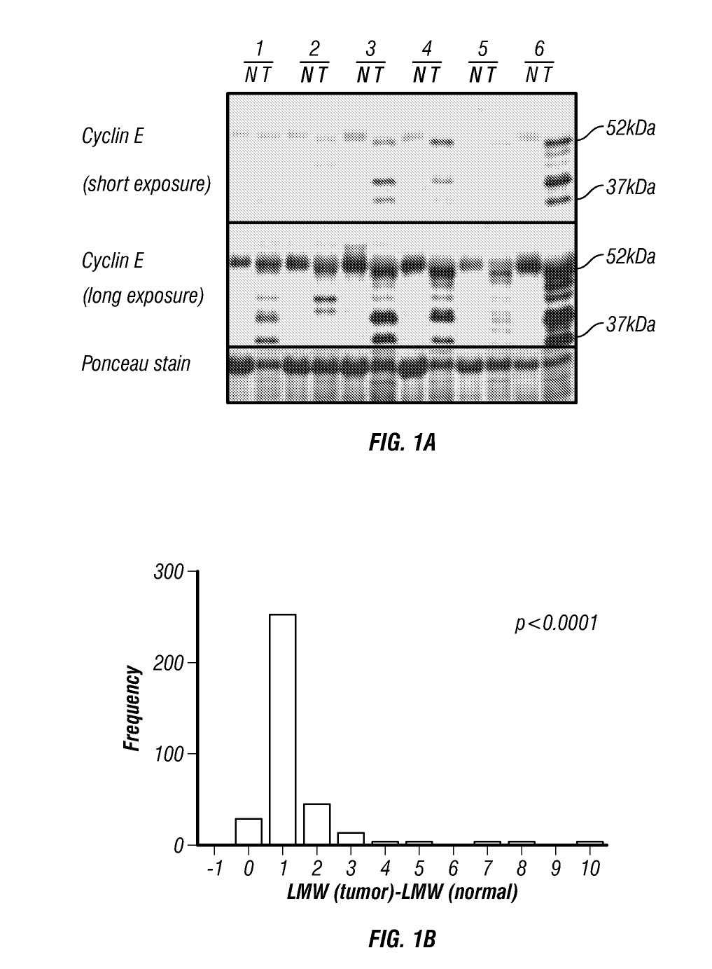

FIGS. 1A-1B show that the LMW isoforms are seen in tumor, but not normal, tissue extracts. As shown in FIG. 1A, lysates were generated from breast tumor and normal adjacent tissue of 6 breast cancer patients and prepared for western blotting. Blots were probed with a cyclin E antibody to the c-terminus to detect the full-length and LMW isoforms of cyclin E and were subjected to short (top panel) and long exposures (middle panel). Ponceau stained bands are shown as a loading control (bottom panel). For each paired sample, the value of LMW cyclin E in the normal sample was subtracted from that of the tumor sample after each was normalized to full-length cyclin E. The histogram (FIG. 1B) shows the frequency of the values of the difference between LMW cyclin E expression in the tumor and normal samples. Positive values indicate more LMW cyclin E in the tumor, whereas negative values indicate more LMW cyclin E expression in the normal samples.

FIG. 1A shows a representative western blot of cyclin E expression in the tumor samples and normal adjacent tissue samples from six patients who are representative of those whose tumors express high levels of LMW-E. The full-length protein was seen in both the paired normal and tumor tissue samples. However, the LMW cyclin E isoforms were seen predominantly in the tumor samples, irrespective of the length of exposure time of the western blots (middle panel).

Densitometric analyses were performed on each of the samples to quantitate both the full-length and LMW cyclin E expression. On average, the frequency of full-length cyclin E expression in the tumor samples was equivalent to that of the normal samples (p=0.058, sign test). However, the frequency of LMW cyclin E expression in the tumor samples was significantly greater than that of the normal tissue samples (p<0.0001, FIG. 1B). These data suggest that the LMW cyclin E forms are processed and occur predominantly in tumor tissue and not normal tissue. With this evidence that LMW cyclin E may be involved in breast cancer development, it became reasonable to examine the biologic consequences of LMW cyclin E expression in vitro and in vivo to see if it is directly linked to the tumorigenic process.

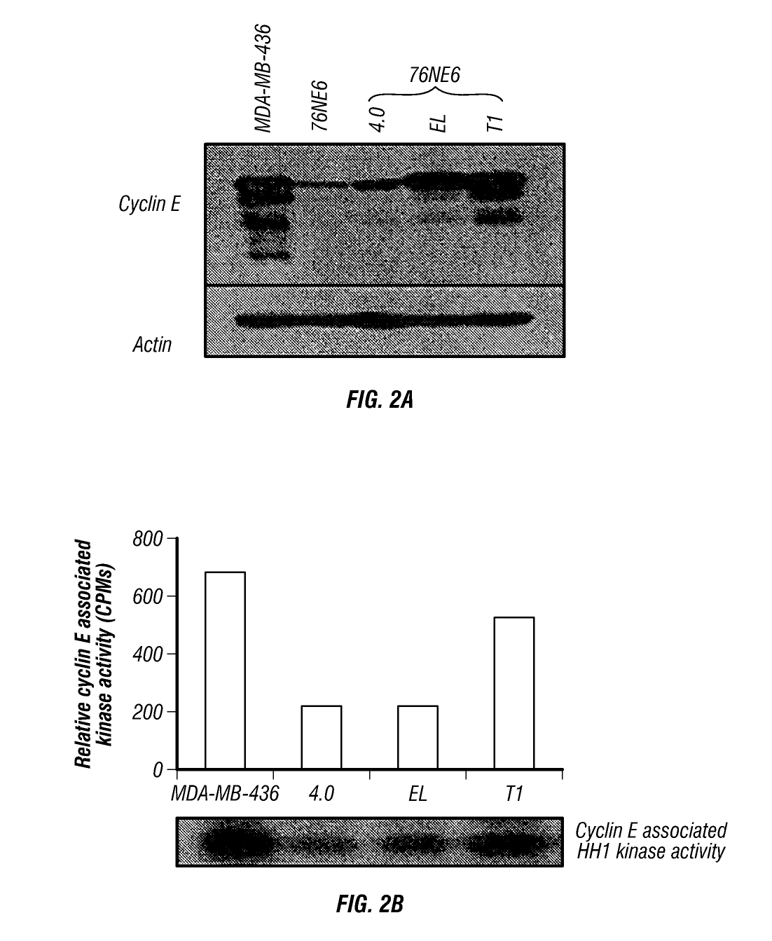

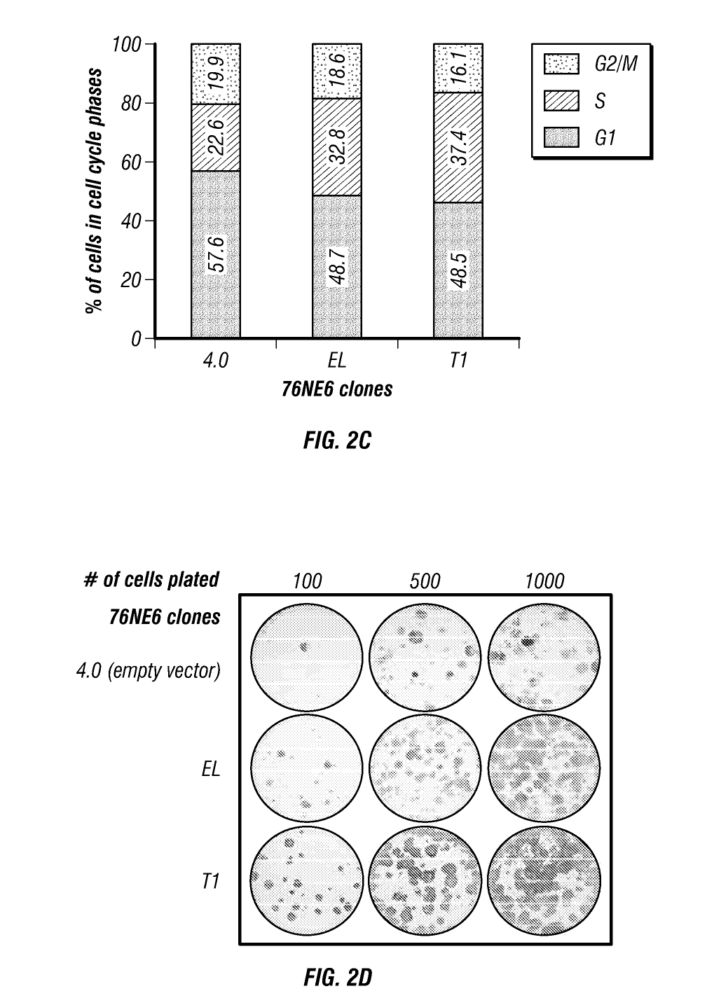

LMW cyclin E expression has biologic consequences. To determine the effects of LMW cyclin E expression in a non-tumorigenic mammary epithelial cell line, stable clones of 76NE6 cells overexpressing full-length (EL) or LMW (T1) isoforms of cyclin E were generated. Western blot analyses showed that the EL and LMW-T1 clones expressed full-length and LMW isoforms respectively, at physiological levels (FIG. 2A) and that the kinase activity associated with the LMW-T1 clones was 2.3 times higher than that of the EL complexes (FIG. 2B). Overexpression of the T1 LMW isoform of cyclin E resulted in up to a 2-fold increase in S phase fraction of cells over the empty vector alone transfectants (FIG. 2C). No significant doubling time differences between the clones overexpressing EL (full-length) as compared with those expressing the T1 LMW cyclin E. However, the T1 clones exhibited a growth advantage in clonogenic assays (FIG. 2D). The LMW-T1 cells form colonies more readily than the EL clones did and both of these clones formed colonies more readily than the empty vector clones did. Therefore, overexpression of the hyperactive LMW isoforms of cyclin E provided a growth advantage to mammary epithelial cells.

FIGS. 2A-2D show that the LMW cyclin E isoforms have biologic characteristics that are unique from that of the full-length cyclin E. Full-length cyclin E (EL) and LMW cyclin E (Ti) was stably expressed in 76NE6 cells along with an empty vector (4.0). As shown in FIG. 2A, a western blot shows the expression of the cyclin E isoforms in the stable clones compared to a breast tumor cell line, MDA-MB-436, and the parental cells. Actin is shown as a loading control. As shown in FIG. 2B, cyclin E immunoprecipitates were subjected to a kinase assay using histone H1 (HH1) as a substrate. The bar graph shows the activity as determined by Cerenkov counting on a scintillation counter for each band of the kinase assay. Flow cytometry was performed to assess the percentage of the stable clones in each phase of the cell cycle (FIG. 2C). Clonogenic assays were performed by plating 100, 500 or 1000 cells from each stable clone and incubating them for 14 days. Colonies were stained with crystal violet (FIG. 2D).

Deregulated cell proliferation is a hallmark of cancer cells and can occur when the cancer cell acquires self-sufficiency in growth signals or a resistance to anti-growth signals (Hanahan and Weinberg, 2000). To determine whether 76NE6 cells expressing LMW cyclin E had a decreased need for supplemental growth factors, LMW-T1 cells, E1 cells and empty vector cells responses to growth factor removal were examined. Growth was arrested in the G0/G1 phase in 100% of the 76NE6 cells with the empty vector and in 64% of the 76NE6-EL cells. In contrast, the 76NE6-T1 cells did not respond to a lack of nutrients by arresting their cell cycle (FIG. 3A). When growth factors were added back to the empty vector and EL cells, they synchronously reentered the cell cycle within the first 10 hours (FIG. 3B), whereas the LMW-T1 cells remained distributed throughout the cell cycle.

To determine whether the 76NE6-empty vector or EL cells were exiting the cell cycle into a G0 phase or arresting in G1 phase while lacking growth factors, Ki67 was measured as a marker of cell cycle activity. Ki67 is expressed in all phases of the cell cycle (G1, S, G2 and M), but not G0. The individual cells were examined for both DAPI (to label DNA of all cells) and Ki67 staining at 0, 24, 72 and 96 hours after growth factor deprivation (FIG. 3B). The percent Ki67 positive cells were examined as a function of DAPI staining over time of growth factor deprivation in the empty vector, EL and T1 overexpressing cells. The staining was quantitated and graphically shown in FIG. 3C. For each time point, the Kruskal-Wallis test was used to compare the fractions of proliferating cells for the 3 conditions (vector alone, EL, Ti). These analyses revealed that there are no significant differences for the 24-hr (P=0.688) and 48-hr (P=0.191) time points, but there are significant differences at the 72-hr (P=0.020) and 96-hr (P=0.013) time points. Next pair wise comparisons, using Mann-Whitney test was used at the 72-hr and 96-hr time points to see where the differences lie. For the 72-hr time point, the difference between EL and T1 approached significance with a P=0.083. For the 96-hr time point, EL is significantly different from T1 (P=0.021). Specifically, in cells transfected with empty vector or EL, only 4.1% and 27% of the cells respectively remained in the cell cycle (as detected by Ki-67 positivity) following 96 hrs of growth factor deprivation. However, the majority, 63%, of T1 transfected cells did not succumb to G0 arrest and remained in active cell cycle (FIG. 3C). These results suggest that the LMW-T1 overexpressing cells circumvented the regulatory mechanism in place in normal cells when challenged with a lack of nutrients and resisted a quiescent state.

As shown in FIG. 3A, FACs analysis using propidium iodide staining was performed on 76NE6 clones (empty vector, EL and Ti) that had been subjected to growth factor deprivation for 120 hours, and harvested every 12 hours (left panel), then followed for 33 hours after growth factor replacement, by harvesting cells every 3 hours (right panel). The percentage of cells in G0/G1 phase of the cell cycle is graphed over time for each of the clones. The stable clones were grown in serum free media for 96 hours, during which time cells were collected (0, 24, 72 and 96 hr) and stained with DAPI (blue, nucleus) and Ki67 (green) (FIG. 3B). The percentage of Ki-67 positive cells was determined for each clone at each time point by counting 4 different slides for each condition (FIG. 3C). For the 72-hr time point, the difference between EL and T1 approached significance with a P=0.083 and shown as one asterisk. For the 96-hr time point, EL is significantly different from T1 (P=0.021) and depicted with two asterisks.

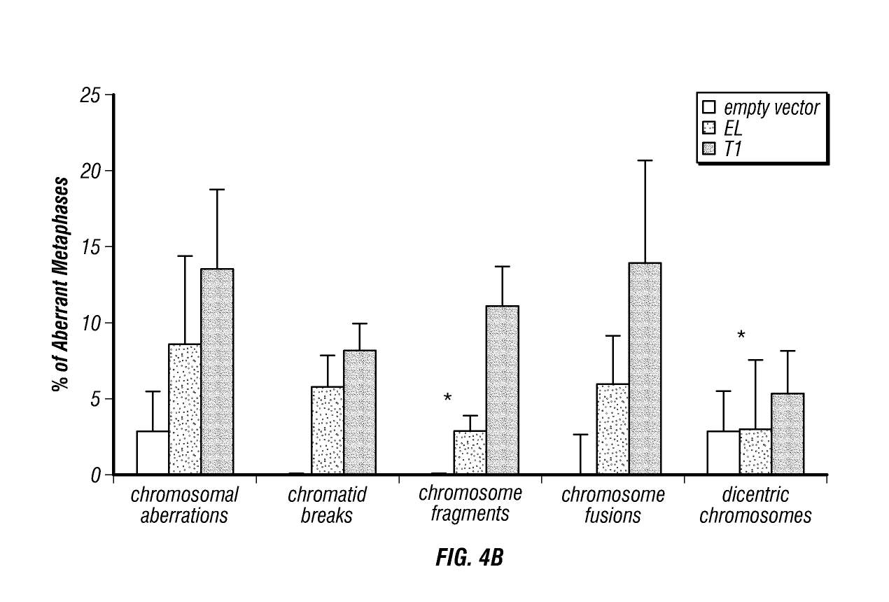

LMW isoforms of cyclin E generate genomic instability. Deregulation of the cell cycle can result in the unfaithful transmission of genetic information manifested as gross chromosomal aberrations. To determine whether deregulation of the cell cycle by cyclin E affects the genomic fidelity of the cell, gross chromosomal aberrations were assessed by karyotype analysis on metaphase arrested 76NE6-cyclin E clones (FIG. 4A). The empty vector karyotype was considered normal with all chromosomes being intact. However, chromosomal aberrations were identified in the karyotypes of both the EL and LMW-T1 cells (FIG. 4A), including: dicentric chromosomes, ring chromosomes, chromatid breaks, chromosome fragments and telomere fusion. FIG. 4B shows the percentage of metaphases that had at least one chromosome with any type of aberration. LMW-T1 overexpressing cells exhibited significantly more overall chromosomal abnormalities than the EL overexpressing cells. The empty vector expressing cells exhibited significantly less (or no) chromosomal aberrations. It can be concluded that 76NE6 cells became genetically unstable subsequent to LMW cyclin E expression.

FIGS. 4A-4B show that the LMW cyclin E isoforms generate genomic instability. Metaphase spreads and karyotype analysis were performed on 76NE6 clones (empty vector, EL or T1). Representative karyotypes of the chromosomes from each clone are shown. Examples of aberrant chromosomes within the metaphase are shown (FIG. 4A). Graph showing the percentage of metaphases with the given aberrations for 3 independent experiments, each of which 32-37 metaphases were analyzed (FIG. 4B). For all types of aberrations assessed, the percentage of metaphases with that aberrancy was significantly different than the other 2 clones, except the two types of aberrancy marked with *, where there was not a significant difference between EL and empty vector cells, statistics were performed using a t-test.

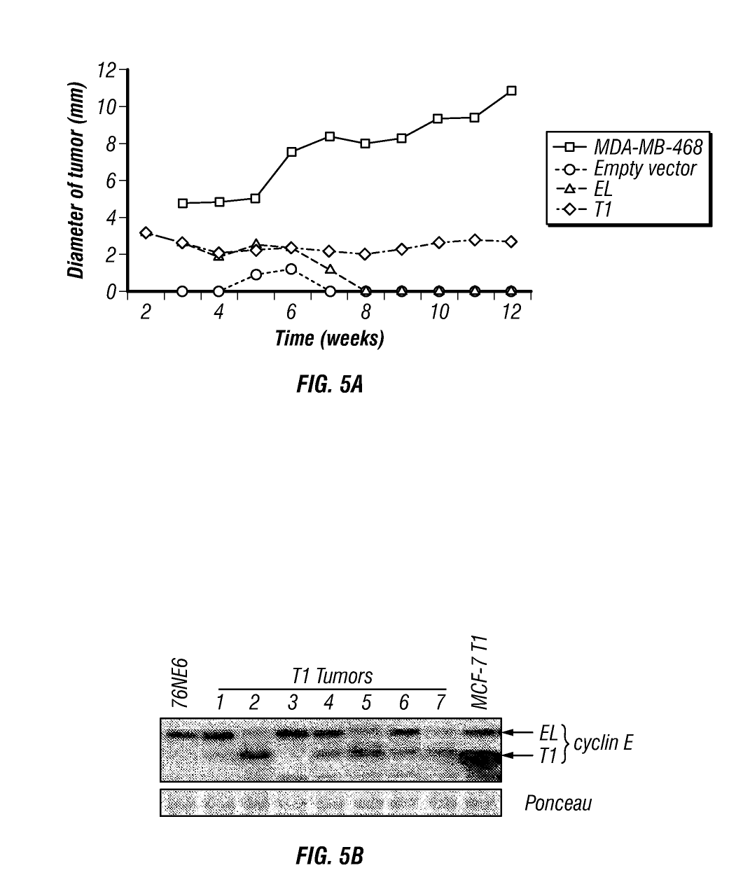

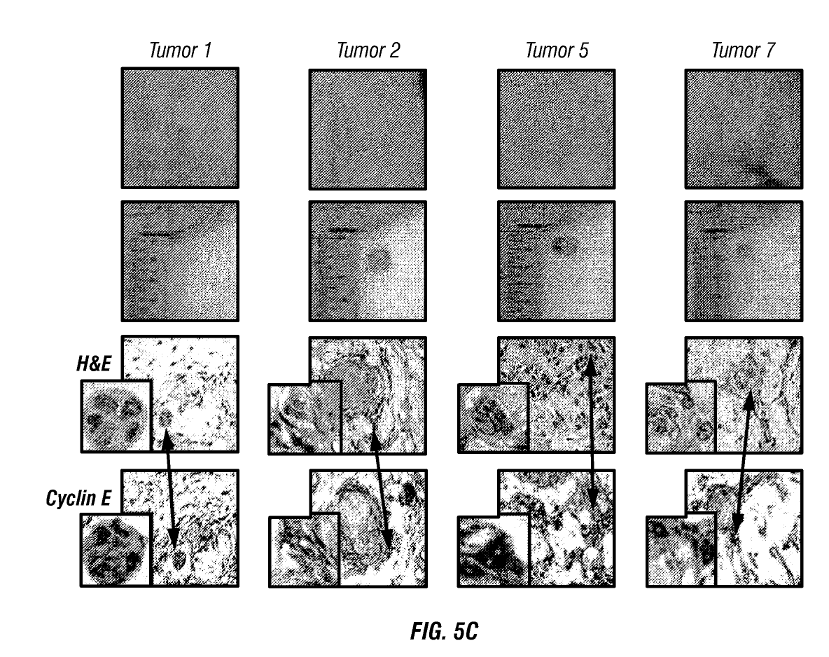

76NE6 cells overexpressing the LMW-T1 cyclin E form tumors in nude mice. The self-sufficiency in growth signals, insensitivity to anti-growth signals (growth factor deprivation) and genomic instability are characteristic of a phenotype transforming from normal to tumorigenic. To test the in vivo tumorigenic potential subsequent to overexpression of LMW cyclin E (T1) in an otherwise non-tumorigenic cell line, the 76NE6 clones (empty vector, EL or LMW-T1) were injected into the mammary fat pad of nude mice. After 3 months, 100% of the mice injected with the 76NE6 LMW-T1 cells had evidence of tumor formation (FIG. 5A). None of the mice injected with the 76NE6 cells expressing EL or vector alone formed tumors. The tumors formed by the LMW-T1 cells were slow growing, averaging 2.7 mm in diameter.

The LMW-T1 tumors were removed and subjected to western blot confirming expression of the LMW-T1 isoform (FIG. 5B) and to pathologic analysis (FIG. 5C). Hematoxylin and eosin staining of clusters of tumor cells present in four representative tumors from the LMW-T1 mice showed pleomorphic nuclei, formation of duct-like structures, and keratinization of cells, suggesting pilar differentiation or adenosquamous carcinomas (FIG. 5C). Furthermore, tumor formation was attributed to the overexpression of cyclin E in the LMW-T1 mice because immunohistochemical staining for cyclin E in these mice showed that cyclin E was strongly expressed in tumor cells from their fat pads (FIG. 5C). From these data it can be concluded that the LMW-T1, but not EL, isoform of cyclin E, when overexpressed in a non-tumorigenic epithelial cell line resulted in tumorigenesis.

FIGS. 5A-5C show that 76NE6 cells overexpressing the LMW-T1, but not EL, isoform of cyclin E form tumors in mice. As shown in FIG. 5A, 1×10⁷ 76NE6 cells overexpressing EL, LMW-T1 or the empty vector or MDA-MB-468 cells (as a positive control) were injected into the mammary fat pads of 10 nude mice (LMW-T1 was injected in to 20 mice). The diameter of the tumor was measured and recorded weekly for each set of 10 mice. The tumors were removed and maintained in tissue culture for inoculation in to a second set of 20 mice (FIG. 5B) Western blots were performed on the 76NE6-LMW-T1 tumors and were probed for cyclin E. As shown in FIG. 5C, photographs were taken of 4 representative 76NE6-LMW-T1 tumors after 12 weeks of growth in the mammary fat pad of nude mice (top panel) and after necropsy (second panel). Slides were made of the 4 representative tumors, and were stained with hematoxylin and eosin (third panel) and subjected to immunohistochemistry with an antibody to cyclin E (bottom panel).

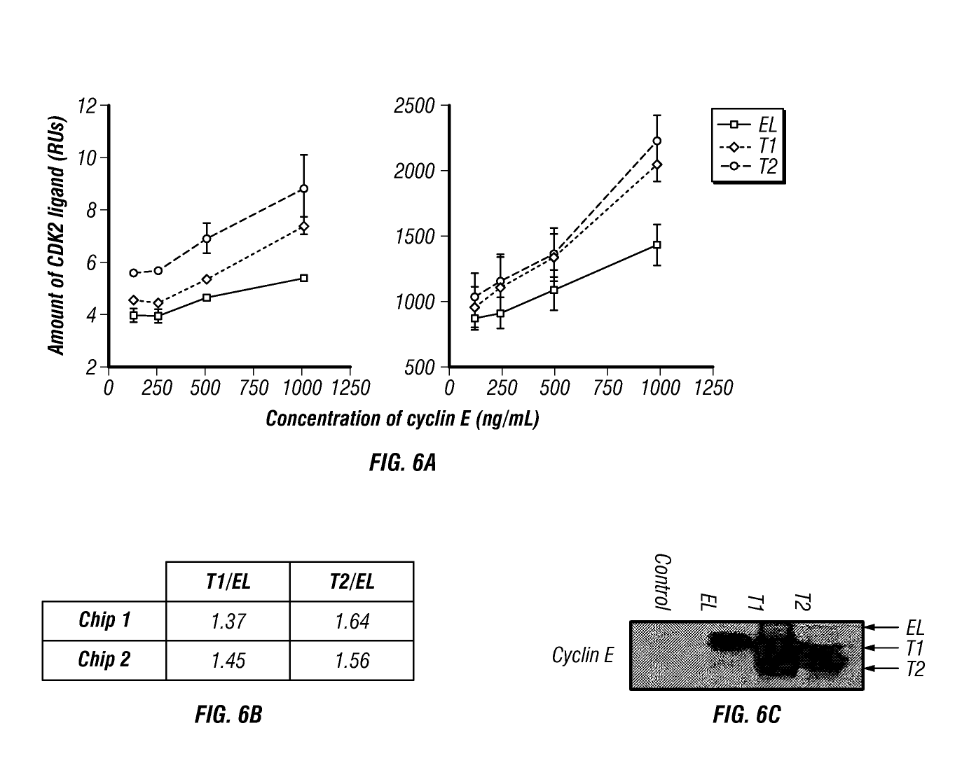

The LMW isoforms of cyclin E are biochemically distinct from the full-length cyclin E. It has been previously shown that the LMW isoforms of cyclin E are hyperactive as a result of increased binding to CDK2 (Wingate et al., 2005). To determine whether objective and quantitative differences exist between the cyclin E isoforms when binding to cyclin E's kinase partner CDK2, which could give rise to the unique biologic functions observed after expression of LMW cyclin E, surface plasmon resonance (SPR) technology was applied using a Biacore 2000 analyzer. FIG. 6A shows the results of 4 different concentrations of the full-length (EL) or LMW (T1 and T2) isoforms of cyclin E isoforms binding to purified CDK2 at a low surface density (˜1370 RUs of CDK2, left graph), and at higher density CDK2 coating (˜8400 RUs, right graph). At all concentrations examined, the highest level of binding to the CDK2 detected was observed when the LMW-T2 isoform was injected, followed by LMW-T1; EL was the lowest. Furthermore, the increased binding by the LMW forms was independent of the amount of CDK2 bound to the chip (compare FIG. 6A right to left panel). The differences in binding to CDK2 became more prominent with increased concentrations of the cyclin E isoforms, with LMW-T1 binding to CDK2 41% more efficiently, and LMW-T2 binding 60% more efficiently than EL (full-length) did (FIG. 6B).

To ensure that the change in refractive index detected and reported by the analyzer was actually due to cyclin E binding to the CDK2, a separate experiment was performed in which each of the analytes was injected over the chip and the bound protein was immediately eluted and subjected to western blot analysis for cyclin E content (FIG. 6C). Cyclin E was present in each of the recovered EL, LMW-T1, and LMW-T2 samples but not in the uninfected lysate control, confirming that the change in refractive index detected was indeed the result of cyclin E binding to the ligand. These data quantitatively show that the hyperactivity associated with the LMW isoforms of cyclin E is due to increased binding efficiency to CDK2. Therefore, the LMW isoforms of cyclin E are biochemically distinct from full-length cyclin E.

As shown in FIGS. 6A-6C, the LMW cyclin E isoforms bind more efficiently than the full-length cyclin E to CDK2. The binding of each cyclin E isoform to CDK2 was assessed using a Biacore 2000 analyzer. The amount of the full-length (EL), LMW (T1 or T2) cyclin E isoforms (4 concentrations) or uninfected sf9 lysate (as a control) bound to CDK2 was determined using a low density (FIG. 6A, left panel) and high density (FIG. 6A, right panel) of immobilized CDK2. The relative amount of T1 and T2 bound to CDK2 compared to EL binding to CDK2 is shown by taking the ratio of the average amount of each isoform bound to CDK2 at the highest concentration (11 μg/mL) of cyclin E (FIG. 6B). Bound protein was recovered and tested on a western blot hybridized with cyclin E antibody (FIG. 6C).

### Discussion:

The LMW isoforms of cyclin E are specifically overexpressed in tumor tissue but not in normal tissue. The frequent appearance of the LMW forms of cyclin E and their correlation with poor prognosis in breast cancer patients suggests that they play specific roles in the development or progression of this malignancy. In this Example, the overexpression of LMW cyclin E led to genomic instability and tumor formation in nude mice. These results illustrate a role for the LMW forms of cyclin E in breast cancer tumorigenesis.

LMW cyclin E perturbs the cell cycle of 76NE6 cells, resulting in an increased percentage of cells in S phase of the cell cycle (FIG. 2C). The ability of LMW-T1 cells to more readily colonize than the EL or empty vector clones in clonogenic assays, suggests that the LMW-T1 clone does acquire a growth advantage compared to the EL (full-length cyclin E) or empty vector clones. The increased percentage of cells in S phase likely represents a delay in S phase, possibly due to the genomic instability observed (FIGS. 4A-4B).

The observed growth advantage could be the result of the LMW-T1 cells being insensitive to anti-growth signals as opposed to increased proliferation. Apparently, LMW cyclin E prevents cells from exiting the cell cycle and entering quiescence upon growth factor deprivation (FIGS. 3A-3C). The cells overexpressing LMW cyclin E remain active in the cell cycle whereas a normal cell senses that the environment is not optimal for cell proliferation. The lack of response by cells overexpressing the LMW isoforms of cyclin E to antigrowth signals could be the result of their resistance to CKIs. Indeed, the accumulation of p27 has been reported to be necessary for cells to enter quiescence (Kiyokawa et al., 1996). Because cells overexpressing the LMW cyclin E isoforms do not enter quiescence under growth factor deprived conditions, it is possible that other cues that typically stop proliferation, such as DNA damage, would be ignored by these cells. Replicating aberrant DNA leads to an unstable genome. Genomic instability has been proposed to be a way in which cells undergo neoplastic transformation because the unstable genome helps the cell acquire the traits of a tumor cell phenotype.

Cells expressing the LMW-T1 isoform had significantly more genomic aberrations than did those expressing full-length cyclin E or empty vector cells. Therefore, it was necessary to determine whether the genomic instability observed in the LMW-T1 overexpressing cells enabled these cells to acquire tumorigenic characteristics. Soft agar colony forming and matrigel invasion assays are commonly used in vitro assays to indicate tumorigenic potential. No significantly increased potential of the LMW-T1 cells to either form colonies in soft agar or invade the matrigel matrix was observed.

However, in the in vivo tumorigenicity assays, all of the mice injected with mammary epithelial cells expressing the LMW-T1 isoform of cyclin E formed tumors, whereas none of the mice injected with the EL or empty vector cells formed tumors. The tumors that formed in the LMW-T1 mice were small, which may be one reason that tumorigenic potential in soft-agar or matrigel was not observed. The tumors formed in this xenograft model (carcinoma with pilar differentiation or adenosquamous carcinomas) had the same pathologic features as the tumors that were previously in the LMW-T1 cyclin E transgenic mouse model (Akli et al., 2007). Of interest, the LMW-T1 transgenic mice went on to develop metastases.

Functional assays confirmed that the cell cycle deregulation observed with expression of LMW cyclin E is secondary to the increased kinase activity associated with these complexes. Aberrant G1 to S phase transitions are a means for tumor cells to facilitate their growth, thus raising the question of how the LMW isoforms achieve their increased kinase activity. The resistance to CKIs by the LMW isoforms of cyclin E is one mechanism by which the LMW isoforms achieve a hyperactive phenotype compared with full-length cyclin E. Another mechanism is the ability of LMW cyclin E to bind to CDK2 more efficiently. It has been thoroughly demonstrated that CDK2 binds to 40% to 60% more of the LMW isoforms than to the full-length cyclin E.

Understanding the mechanisms of LMW cyclin E in tumor formation is critical because patients with breast cancers that overexpress LMW cyclin E have an extremely poor outcome. Therefore, LMW cyclin E may serve as a novel target for therapeutic intervention, specifically targeting the formation of the LMW isoforms of cyclin E or their increased kinase activity.

### Example 2

### A Novel Interaction Between HER2 and Cyclin E in Breast Cancer

Here is presented an examination of the functional relationship between HER2 and cyclin E in breast cancer patients and tumor cells, and a demonstration that their functions are interlinked, suggesting that a treatment strategy targeting both proteins may be better than targeting either one alone in patients whose tumors overexpress both HER2 and LMW cyclin E.

### Materials and Methods

Cell Lines MCF-7, SKBr3, and BT474 breast cancer cells were obtained from American Type Culture Collection (Manassas, Va.). MCF-7-HER-18 cells were a gift from Dr. Mien-Chie Hung (The University of Texas M. D. Anderson Cancer Center, Houston, Tex.). All four cell lines were cultured and maintained in Dulbecco's modified Eagle's medium (DMEM) supplemented with 10% fetal bovine serum (FBS), 2 mM L-glutamine, 100 U/mL penicillin, and 100 μg/mg streptomycin (Gibco; Invitrogen Corp, Grand Island, N.Y.). Media for MCF-7-HER-18 cells also included 5 μg/mL G418. All cells were maintained in a humidified atmosphere of 5% CO₂ at 37° C.

Plasmids and siRNA FLAG-tagged constructs corresponding to the full-length or N-terminal truncated cyclin E and the different LMW isoforms were generated as previously described (Harwell et al., 2000). These isoforms included cyclin EL (ELI), T1 (EL2/EL3), and T2 (EL5/6). MCF-7 cells stably overexpressing each of these isoforms were generated as previously described (Akli et al., 2004). Validated HER2/neu siRNA (ID#540) and Silencer negative control #1 siRNA were purchased from Ambion (Austin, Tex.).

siRNA Transfections MCF-7, MCF-7-HER-18, SKBr3, and BT474 cells were transiently transfected with the predesigned siRNA to the HER2/neu gene. Briefly, 5×10⁴-1×10⁵ cells/well were plated in 6-well plates 24 hours before transfection. Five-hundred microliters of opti-MEM medium (Gibco; Invitrogen, Carlsbad, Calif.) and 12.5 μL of 50 μM HER2 siRNA were mixed for 10 minutes at room temperature. Simultaneously, 500 μL of opti-MEM medium and 7.5 μL of X-treme GENE siRNA transfection reagent (Roche Applied Science, Base1, Germany) were mixed for 10 minutes at room temperature. The two mixtures were then combined for an additional 20 minutes. Two hundred microliters of the combined mixture were then added to designated wells. A mixture containing negative control siRNA was prepared in an identical manner and added to duplicate wells of the 6-well plates. Seventy-two hours after transfection, cells were harvested for analysis by flow cytometry, western blot, kinase assay, and real-time quantitative polymerase chain reaction (qRT-PCR).

Treatment With Trastuzumab The anti-HER2 monoclonal antibody trastuzumab (Herceptin, Genentech, San Francisco, Calif.) was reconstituted in normal saline to obtain a stock solution with a concentration of 21 mg/mL. This was subsequently diluted in DMEM supplemented with 10% FBS, 2 mM L-glutamine, 100 U/mL penicillin, and 100 μg/mg streptomycin to concentrations of 10 μg/mL and 20 μg/mL. Twenty-four hours after 1×10⁶ cells were plated on 100 mL plates, media were changed to low-serum media, DMEM with 0.5% FBS for MCF7, SKBr3, or BT474 cells and, for MCF-7-1 {ER-18 cells, DMEM with 0.5% FBS and 0.5 μg/mL G418. After another 24 hours (48 hours after plating), cells were treated with different concentrations of trastuzumab for 48 hours, at which point cells were either harvested and subjected to flow cytometric analysis or lysates were obtained for western blot analysis or kinase assays.

Confocal Immunofluorescence Microscopy Chamber slides were placed into 6-well plates, after which cells were plated at 4×10⁵ cells/well overnight and then transfected with HER2 siRNA or treated with trastuzumab as described above. Seventy-two hours after transfection with siRNA or 48 hours after treatment with trastuzumab, cells were stained with different antibodies, washed with PBS, permeabilized with 0.2% Triton X-100 for 20 minutes at 4° C., blocked with 1% normal goat serum for 1 hour, and incubated with a primary antibody overnight at 4° C. Primary antibodies included the monoclonal HER2/neu antibody (Cell Signaling, Danvers, Mass.) and polyclonal cyclin E, p21, and p27 antibodies (Santa Cruz Biotechnology Inc, Santa Cruz, Calif.), all at a 1:200 dilution. The following day, cells were incubated for 1 hour with a secondary antibody: fluorescein isothiocyanate-conjugated goat anti-mouse IgG was used for cells that had been treated with monoclonal antibodies, and rhodamine-conjugated goat anti-mouse IgG was used for cells treated with polyclonal antibodies. After incubation with secondary antibodies, cells were washed, and TO-PRO-3-iodide (10 μL/slide) was added as a nuclear stain. Cells were then visualized using the confocal immunofluorescence microscope (Olympus FV 500 confocal microscope, Melville, N.Y.) with a 40× oil immersion lens. The multi-line argon laser was used to simultaneously stimulate green and red fluorescence, after which final images were obtained by merging the images from the green and red channels.

Western Blot Analysis and Immunoprecipitation Kinase Assays Cell lysates were prepared and subjected to western blot analysis as previously described (Rao et al., 1998). The primary antibodies used were HER2 (Cell Signaling), cyclin E (Santa Cruz Biotechnology Inc., Santa Cruz, Calif.), p21 (0P64; Oncogene Research Products, Boston, Mass.), and p27 (Transduction Laboratories, Lexington, Ky.), all at 1 μg/mL. After incubation with primary antibodies, blots were washed and then incubated in BLOTTO with secondary antibodies at a dilution of 1:5000 for 1 hour at room temperature. Membranes were developed and protein signals detected by using chemiluminescence western blotting detection reagents (Amersham Biosciences, Buckinghamshire, England). Membranes were incubated with antibody against β-actin (Santa Cruz Biotechnology) to assess equal protein loading. Lysates from MDA-MB-436 and MDA-MB-435 (obtained from ATCC) were used as positive controls for low molecular weight cyclin E. Lysates from 76NE6 mammary epithelial cells (a gift from Dr. Vimla Band, University of Nebraska Medical Center, Omaha, Nebr.) were used as a negative control for low molecular weight cyclin E. Kinase assays using cyclin E polyclonal antibody were performed as previously described (Porter et al., 2001). For quantitation of relative kinase activity, the bands corresponding to histone H1 were analyzed on a phosphoimager Typhoon 9400 machine (Amersham Biosciences, Sunnyvale, Calif.).

Cell Proliferation Assays Cell viability, as a measure of proliferation, was determined by counting cells using 3-[4,5-dimethylthiazol-2-yl]-2,5-diphenyl tetrzolium bromide (MTT; Sigma, St. Louis, Mo.) assays. Briefly, 1000 cells/well were seeded in triplicate in 96-well plates, after which they were transfected with HER2 siRNA or treated with various doses of trastuzumab. After 72 hours, cells were fixed with dimethyl sulfoxide and stained with 5 mg/mL MTT solution. The absorbance was read with an automated spectophotometric miniplate reader (EL808 Ultramicroplate reader; Bio-Tek instruments, Inc., Winooski, Vt.) at 570 nm. Values were normalized and plotted as the percentage change relative to counts of the control cells (mean±standard error of the mean [SEM]).

RNA Extraction and cDNA Synthesis By Reverse Transcription Total cellular RNA from cell lines was extracted and isolated using the Qiagen RNeasy kit (Qiagen Inc., Valencia, Calif.). Briefly, cells were trypsinized, harvested, and then lysed and homogenized in the presence of a guanidine-thiocyanate-containing buffer to inactivate RNases. Ethanol was added to provide appropriate binding conditions, and the samples were applied to an RNeasy Mini spin column (Qiagen), which allowed for the binding of total RNA to the membrane. RNA was eluted in 30 μL of water, and purified RNA was quantified by spectrophotometry (Nanoprop ND-1000; Nanoprop Technologies, Wilmington, Del.). Synthesis of cDNA was performed from 1 μg of total RNA at a total volume of 20 μL using the Roche Transcriptor First Strand cDNA Synthesis kit (Roche Applied Science, Indianapolis, Ind.). All reverse transcriptase reactions were carried out with anchored oligo(dT)18 primers to target the transcription of polyadenylated mRNA and to generate full-length cDNAs. The resulting cDNA was frozen at −80° C. until it was used in qRT-PCR.

Quantitative Real-Time Polymerase Chain Reaction RT-PCR reactions were performed on a Rotor-Gene 2000 Real-Time cycler (Corbett Research, Sydney, Australia). cDNA samples were diluted with autoclaved water to a volume of 250 μL to obtain a concentration of 1 μg/μL. Volumes of 4.5 μL were used for PCR. The primer sequences for cyclin E (forward primer 5′-TTCTTGAGCAACACCCTCTTCTGCAGCC-3′ (SEQ ID NO:2)), (reverse primer 5′-TCGCCATATACCGGTCAAAGAAATCTTGTGCC-3′ (SEQ ID NO:3)) yielded a 138-bp product. f3-Actin was used as an endogenous control. The primer sequences for 13-actin (forward primer 5′-TCACCCACACTGTGCCCATCTACGA-3′ (SEQ ID NO:4)), (reverse primer 5′-TGAGGTAGTCAGTCAGGTCCCG-3′ (SEQ ID NO:5)) yielded a 155-bp product. Primers were obtained from Integrated DNA Technologies, Inc (Coralville, Iowa). Primers were received lyophilized and brought up to a stock solution of 100 μM with autoclaved water. Prior to use, primers were diluted to 2 μM. PCR products were detected using SYBR Green Jumpstart Taq Ready Mix (Sigma, St. Louis, Mo.). For each reaction, 0.25 μL of each primer was mixed with 5 μL of the SYBR green mix. All reactions were performed in triplicate. Data analysis was performed using Rotor-Gene Analysis software, version 5.0 (Corbett Research). Expression levels of cyclin E were normalized to 13-actin expression levels.

Animal Studies Nude mice were obtained from Charles River Laboratories (Wilmington, Mass.) and injected in the posterior cervical region with 0.5-mg estrogen pellets. Two days later, 5×10⁶ MCF-7-HER-18 cells were injected into the thoracic mammary fat pad. When tumors reached 100 mm³, mice were divided into two groups that received treatments twice weekly for 3 weeks: group 1 (n=5) animals received injections of PBS; group 2 (n=5) animals received 110 μg of trastuzumab by intraperitoneal injection. Tumor diameter and volume were measured and recorded twice a week until the animals were euthanized. All animals were cared for and euthanized according to institutional guidelines.

Study Patients The clinical data from 395 breast cancer patients were previously reported by Keyomarsi et al. (2002).

Statistical Analysis Disease-specific survival (DSS) was calculated from the date of surgical excision of the primary tumor to the date of death or last follow-up. Data for patients who died from causes other than breast cancer were censored at the time of death. DSS survival curves were computed by the Kaplan-Meier method. (Kaplan and Meier, 1958) Univariate analyses of DSS survival according to levels of HER2, total and LMW cyclin E were performed with the use of a two-sided log-rank test. (Cox, 1972) Continuous data obtained from experiments analyzing cell cycle profiles and RT-PCR reactions was compared using the paired, 2-tailed Student's t-test. Probabilities <0.05 were considered significant

### Results

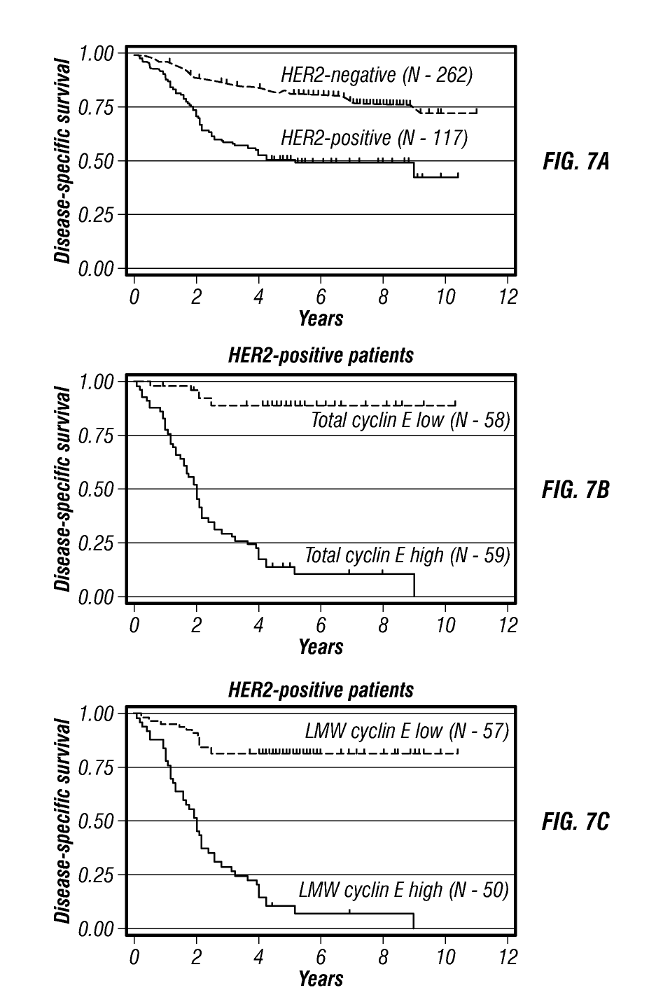

Patients with HER2-Overexpressing Tumors and High Levels of Cyclin E Have A Worse 5-Year Disease-Specific Survival To determine if a clinically relevant relationship exists between HER2 and cyclin E, data was analyzed from a cohort of 395 breast cancer patients that had originally been studied to determine the relationship between total (full-length+LMW) cyclin E expression and survival (Keyomarsi et al., 2002). The 5-year DSS rates were significantly worse in patients with HER2— positive tumors than in those with HER2-negative tumors (P<0.0001) (FIG. 7A). Next, patients with HER2-positive tumors (n=117) were stratified by total cyclin E levels. Patients with HER2-positive tumors and high levels of total cyclin E (n=59) had a 5-year DSS of 14% compared to 89% in patients with low levels of total cyclin E (n=58; P<0.0001; FIG. 7B). When stratified only by LMW cyclin E levels, there was again a significant difference between subgroups. Patients with high levels of LMW cyclin E (n=50) had a 5-year DSS of 10%; patients with low levels of LMW cyclin E had a 5-year DSS of 82% (n=67; P<0.0001; FIG. 7C). These data demonstrate that the overexpression of both HER2 and cyclin E, particularly LMW cyclin E, contributes to an aggressive phenotype of breast cancer.

FIGS. 7A-7C show results indicating a relationship between HER2 and cyclin E in breast cancer patients. A cohort of 395 breast cancer patients previously described by Keyomarsi et al. was reanalyzed to discern any correlation between HER2 and cyclin E levels. HER2 status was known in 379 (96%). As shown in FIG. 7A, the Kaplan-Meier estimate of disease-specific survival (DSS) was significantly higher for patients with HER2-negative tumors (n=262) than for patients with HER2-overexpressing tumors (n=117; P<0.0001). Subset analysis of patients with HER2-overexpressing tumors stratified by total cyclin E expression demonstrates a significantly worse median DSS in patients with high total cyclin E levels (median DSS, 2 years) than in those with low cyclin E levels (median DSS not reached during 10-year follow-up period; P<0.0001 (FIG. 7B). Further stratification of patients by levels of low molecular weight (LMW) cyclin E demonstrates a significantly worse median DSS in patients with high LMW cyclin E levels (median DSS, 2 years) than in those with low LMW cyclin E levels (median DSS not reached during 10-year follow-up period; P<0.0001 (FIG. 7C). Cyclin E expression was measured by western blot analysis. Total cyclin E was obtained by adding together the full-length and LMW cyclin E values and dichotomizing at levels of cyclin E (1.2 versus <1.2).

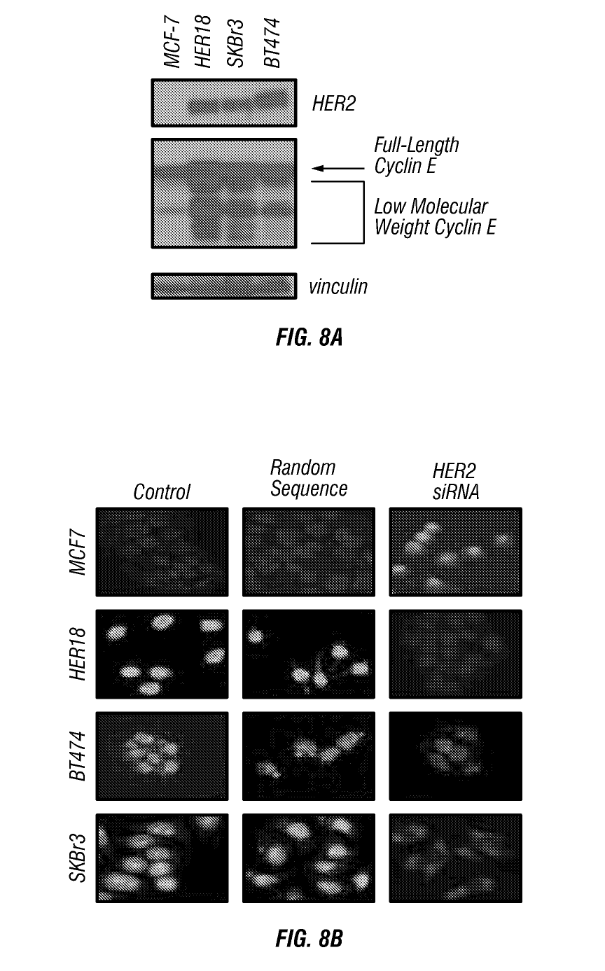

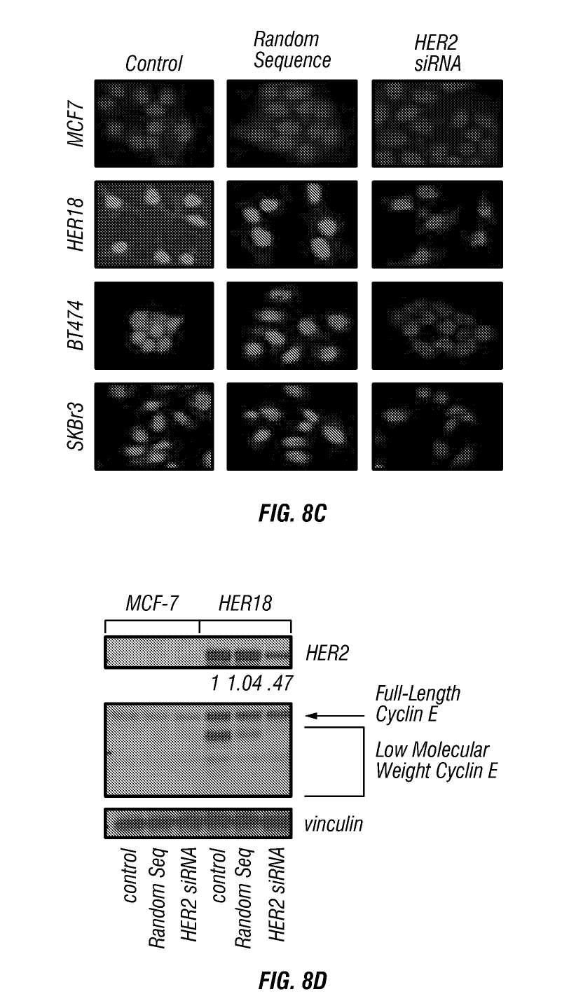

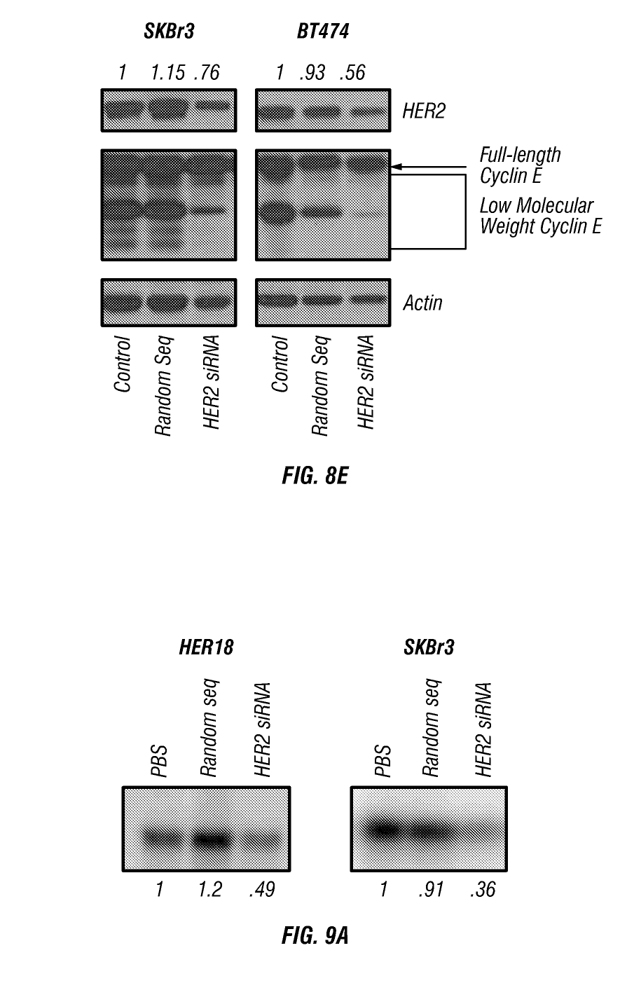

HER2/neu Expression Alters Cyclin E Expression Activation of the HER2/neu signal transduction pathway promotes cellular proliferation through the shortening of the G1 phase of the cell cycle (Timms et al., 2002). Similarly, the overexpression of cyclin E shortens the length of the G1 phase. Therefore, one hypothesis is that HER2 overexpression may modulate cyclin E expression or activity. To this end, the HER2-overexpressing breast cancer cell lines MCF-7-HER-18, SKBr3 and BT474 were transfected with HER2/neu siRNA. MCF-7-HER-18, SKBr3 and BT474 express full-length and LMW forms of cyclin E. MCF-7, the parental cell line of MCF-7-HER-18, is a low-HER2 expressing cell line that expresses full-length cyclin E but negligible levels of LMW cyclin E (FIG. 8A). Immunofluorescence confocal microscopy revealed that transfection with HER2 siRNA decreased HER2 and cyclin E expression (FIGS. 8B-8C). Furthermore, western blot analysis of lysates obtained from HER2 siRNA—transfected cells revealed that the decrease in cyclin E after HER2 knockdown was primarily due to a decrease in expression of LMW isoforms (FIGS. 8D-8E). To determine if a feedback loop and ongoing crosstalk exist between HER2 and cyclin E, HER2 expression in MCF7 cells engineered to overexpress either the full-length or one of the LMW isoforms of cyclin E was examined. As shown by the supplementary data, there were no changes in HER2 expression or its phosphorylated form, suggesting that cyclin E lies downstream of HER2.

FIGS. 8A-8E show the Effect of HER2 downregulation on cyclin E expression. HER2-overexpressing breast cancer cell lines were transfected with HER2 siRNA to determine the effect of HER2 downregulation on cyclin E expression. As shown in FIG. 8A, HER2-overexpressing breast cancer cell lines MCF-7-HER-18, SKBr3, and BT474 were grown exponentially. MCF-7 cells were used as a HER2 negative control. Lysates obtained from these cells were subjected to western blot analysis using 50 μg of protein for each cell line in each lane of either a 7% (HER2) or 10% (cyclin E) sodium dodecyl sulfate—polyacrylamide gel electrophoresis (SDS-PAGE) gel. Vinculin was used as a loading control. As shown in FIG. 8B, HER2-overexpressing MCF-7-HER-18, SKBr3, and BT474 tumor cells were transfected with HER2 siRNA. After 72 hours, confocal immunofluorescence microscopy with antibodies against HER2 (green) demonstrated knockdown of HER2 in cells transfected with BER2 siRNA, compared to mock transfected control cells or those transfected with random sequence siRNA. TO-PRO-3-iodide (blue) was used as a nuclear stain. MCF-7 cells, which do not overexpress HER2, were used as a negative control. Confocal immunofluorescence microscopy using the C-19 antibody against cyclin E (red) showed a decrease in total cyclin E expression in HER2-overexpressing MCF-7-HER-18, SKBr3, and BT474 cells that had decreased HER2 expression after transfection with HER2 siRNA (FIG. 8C).

As shown in FIG. 8D, after optimization of HER2 siRNA transfection conditions, HER2-overexpressing MCF-7-HER-18 cells were again transfected with HER2 siRNA. Lysates harvested from these cells were used to perform western blot analysis probing for HER2 and cyclin E. Western blot demonstrated a decrease in HER2 expression with a concomitant decrease in cyclin E expression, primarily a decrease in the LMW forms. MCF-7 was used as a HER2-negative control. As shown in FIG. 8E, Western blot confirmed HER2 knockdown after transfection with HER2 siRNA in SKBr3 and BT474, two breast cancer cell lines with endogenous HER2-overexpression. Densitometry was performed to quantitate HER2 expression. Western blot with HE-12 antibody against cyclin E revealed decreased LMW cyclin E in HER2 siRNA transfected cells compared to controls confirming the differential effect on full-length versus LMW cyclin E.

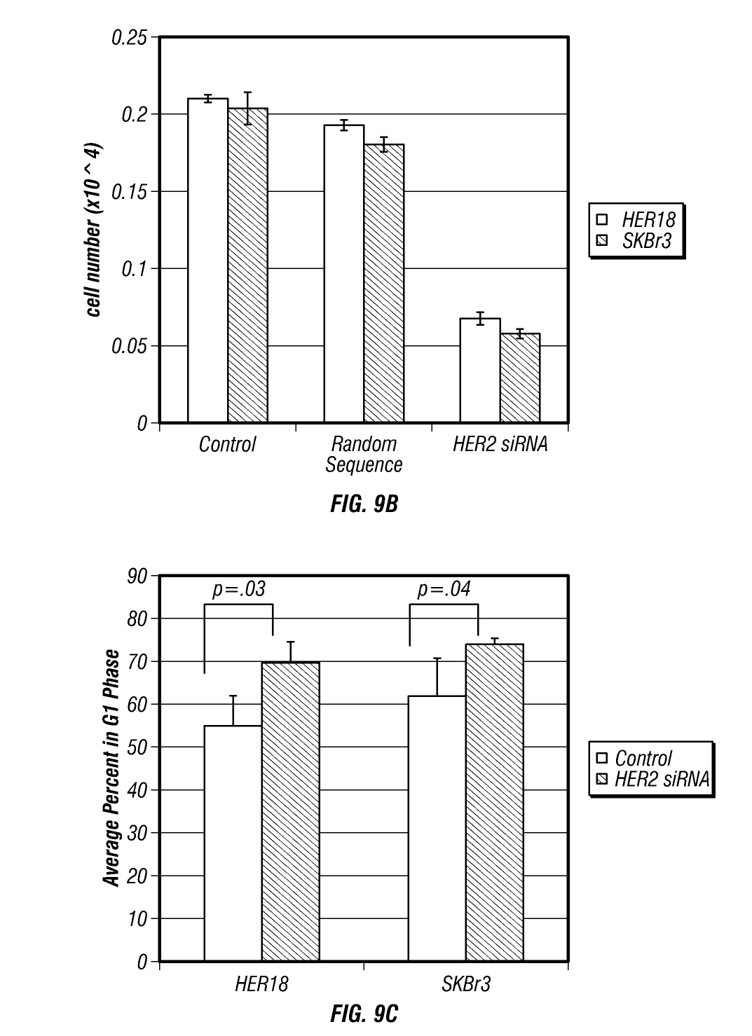

HER2Downregulation Results In Decreased Cyclin E Activity and Accumulation in G1. To assess the effect of HER2 signaling on cyclin E, HER2 levels were altered and then examined the modulation of cyclin E-associated kinase activity. HER2-overexpressing MCF-7-HER-18 and SKBr3 cells were transfected with HER2 siRNA and subjected to cyclin E-associated kinase assay using histone H1 as the substrate. As shown in FIG. 9A, HER2 downregulation decreased cyclin E—associated kinase activity. This effect was more pronounced in SKBr3 cells, which had a 64% decrease in cyclin E-associated kinase activity after HER2 siRNA transfection. In contrast, MCF-7-HER-18 cells, which have higher baseline levels of LMW cyclin E than SKBr3 cells, had a 51% decrease in cyclin E—associated kinase activity. These data suggest that the extent of decrease in cyclin E-associated kinase activity is related to baseline levels of LMW cyclin E expression Since the functionality of cyclin E is crucial in the G1/S transition.

The effect of HER2 downregulation and subsequent decrease in LMW cyclin E expression was examined on proliferation and on the regulation of the G1/S transition. After transfecting MCF-7-HER-18 and SKBr3 cells with HER2 siRNA (FIG. 9B), a pronounced decrease in proliferation and a concomitant increase in the percentage of cells in the G1 phase of the cell cycle (FIG. 9C) was observed. In three duplicate experiments, the mean±SEM percentage of MCF-7-HER-18 and SKBr3 cells in G1 increased by 27.3%±7.4% (P=0.03) and 20.7%±7.6% (P=0.04), respectively. These data demonstrate that decreased LMW cyclin E expression after HER2 knockdown contributes to an increased accumulation of cells in G1.

FIGS. 9A-9C show the Effect of HER2 downregulation on cyclin E-associated kinase activity and cell cycle profiles. To investigate the effects of HER2 downregulation on cyclin E function, kinase assays and fluorescence-activated cell sorting (FACS) analysis of cell cycle profiles were performed after cells were transfected with HER2 siRNA. As shown in FIG. 9A, HER2-overexpressing MCF-7-HER-18 and SKBr3 tumor cells were transfected with HER2 siRNA. Control cells were mock transfected or transfected with random sequence siRNA. Following transfection, equal amounts of protein (250 μg) from cell lysates were immunoprecipitated with an anti-cyclin E antibody and protein G-sepharose beads using Histone H1 as a substrate. For each cell line, bands corresponding to Histone H1 phosphorylation were quantitated through phosphoimaging. As shown, cells transfected with HER2 siRNA had decreased cyclin E-associated kinase activity compared to control cells in both the MCF-7-HER-18 and SKBr3 cell lines. The decrease in cyclin E-associated kinase activity was greater in the SKBr3 cell line, which has lower levels of LMW cyclin E expression than the MCF-7-HER-18 cell line. As shown in FIG. 9B, cell viability as a measure of proliferation was assessed using an MTT assay performed on MCF-7-HER-18 and SKBr3 tumor cells, 72 hours after the cells were transfected with HER2 siRNA. Mock transfected cells and MCF-7-HER-18 and SKBr3 cells transfected with random sequence siRNA were used as controls. The number of tumor cells transfected with HER2 siRNA was smaller than the number of cells that were mock transfected or transfected with random sequence siRNA, suggesting that HER2 knockdown resulted in decreased proliferation. As shown in FIG. 9C, to determine if an alteration in cell cycle distribution contributed to the decrease in proliferation, cell cycle profiles were determined using FACS analysis of MCF-7-HER-18 and SKBr3 tumor cells that had been transfected with HER2 siRNA. Experiments were repeated in triplicate, and the mean percentage of cells in the G1 phase for mock transfected controls versus siRNA-transfected MCF-7-HER-18 and SKBr3 tumor cells is shown. Error bars represent the standard error of the mean. For both cell lines, there was a significant increase in the percentage of cells in the G1 phase after HER2 was knocked down using HER2 siRNA.

Effect of HER2 on Cyclin E Expression Is Post-Transcriptional To determine if HER2 siRNA-mediated downregulation of cyclin E is transcriptionally regulated, RNA extracted from MCF-7-HER-18 and SKBr3 cells transfected with HER2 siRNA was subjected to qRT-PCR, after which a comparative quantitation analysis was performed using J3-actin as an endogenous reference gene. In three duplicate experiments performed on MCF-7-HER-18 cells, the mean cyclin E/P-actin mRNA ratios were 0.006, 0.009 (P=0.18), and 0.013 (P=0.18) for controls, cells transfected with random sequence siRNA, and cells transfected with HER2 siRNA, respectively (Table 1A). Experiments were repeated in triplicate using SKBr3 cells, and the mean cyclin E/Pactin ratios were 0.157, 0.288 (P=0.46), and 0.128 (P=0.24) (Table 1B).

TABLES 1A-1B
(A)

| | | Random | HER2 |
|---|---|---|---|
| | Control | Sequence | siRNA |
| Transfection #1 | .005 | .007 | .009 |
| Transfection #2 | .007 | .009 | .018 |
| Transfection #3 | .007 | .011 | .016 |

(B)

| | | Random | HER2 |
|---|---|---|---|
| | Control | Sequence | siRNA |
| Transfection #1 | .044 | .053 | .073 |
| Transfection #2 | .084 | .351 | .039 |
| Transfection #3 | .342 | .458 | .272 |

No significant differences were found between the levels of cyclin E mRNA in cells transfected with HER2 siRNA and control cells either left untreated or transfected with random sequence siRNA, suggesting that HER2 does not regulate cyclin E transcription. This supports the finding that the effect of HER2 is greatest on the LMW isoforms of cyclin E, which are the result of a post-translational modification of the full-length protein.

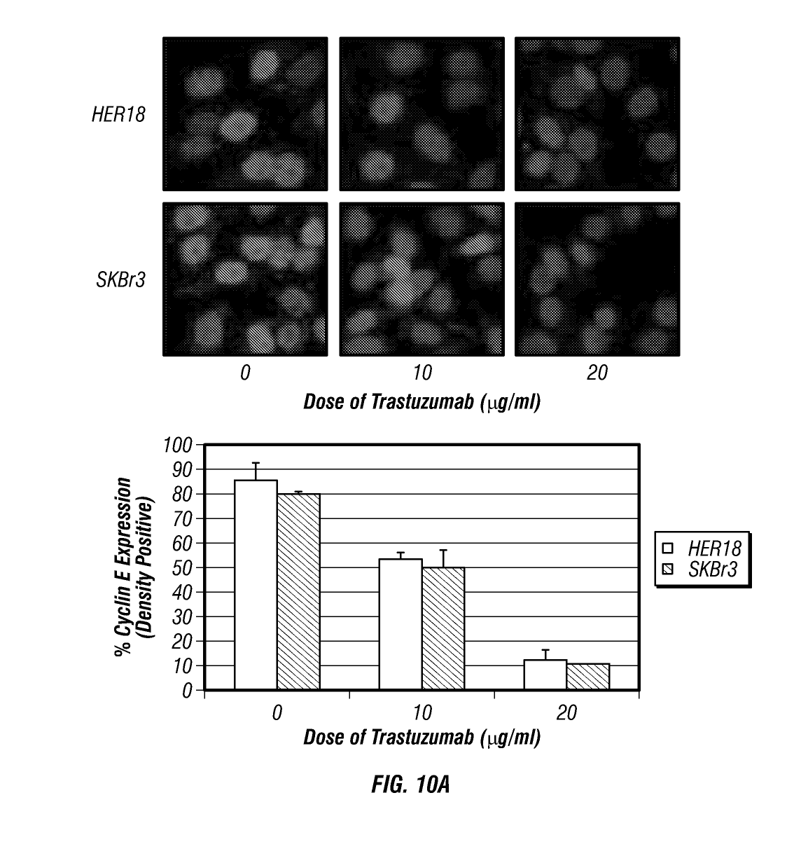

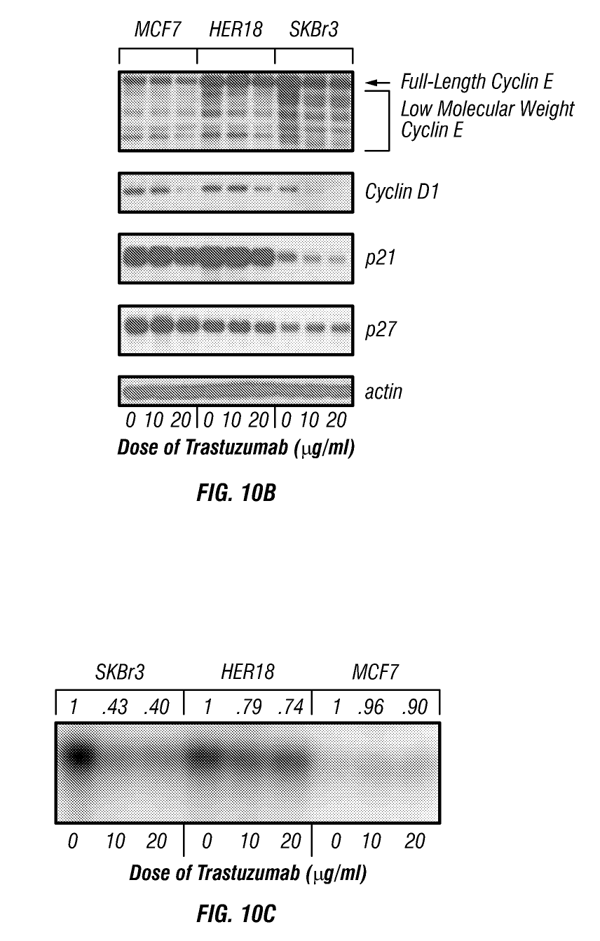

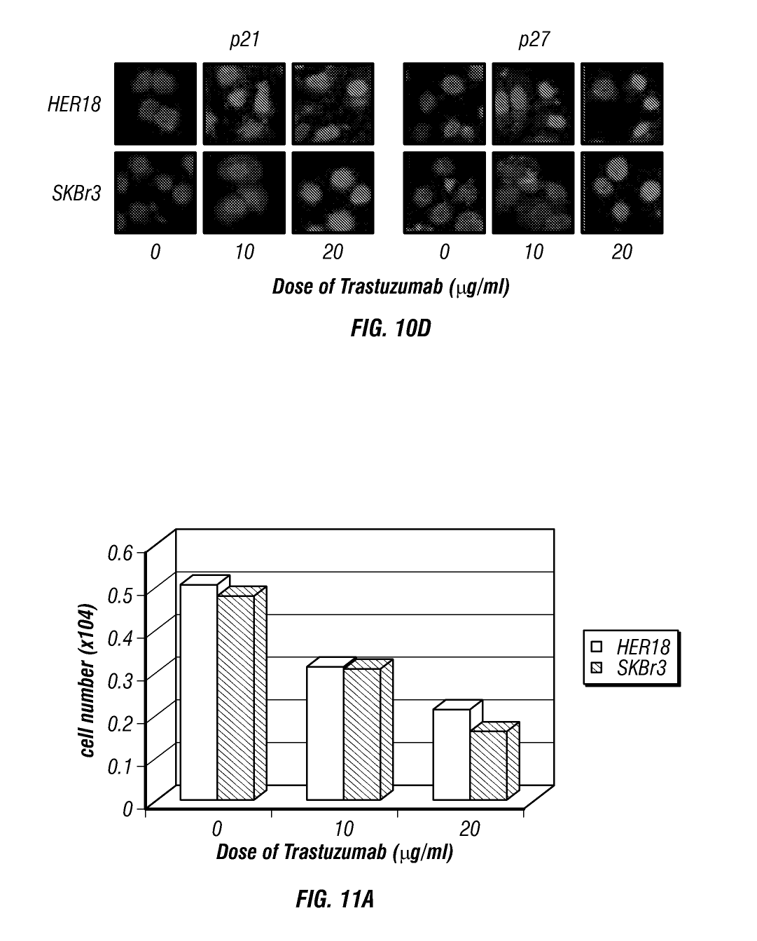

Decreased HER2-Mediated Signaling Results in Decreased Cyclin E Expression and Altered Expression and Localization of G1 Regulators. Based on the results described thus far, it was hypothesized that cyclin E lies downstream of HER2-mediated signaling cascades and that decreased activity through these pathways affects cyclin E expression and activity. Using immunofluorescence confocal microscopy to assess cyclin E expression, a dose-dependent decrease was observed in cyclin E expression in MCF-7-HER-18 and SKBr3 cells treated with different doses of trastuzumab. In particular, cyclin E expression in MCF-7-HER-18 and SKBr3 cells treated with 20 μg/mL trastuzumab decreased by 86.5% and 86.8%, respectively, compared to levels in untreated cells (FIG. 10A). Additionally, western blot analysis confirmed that the decrease was primarily due to a decrease in expression of the LMW isoforms (FIG. 10B). Similar to the effect seen after transfection with HER2 siRNA, decreased HER2-mediated signaling due to trastuzumab treatment resulted in decreased cyclin E-associated kinase activity (FIG. 10C). Consistent with the results from the kinase assays performed on HER2 siRNA-transfected cell lysates shown in FIGS. 9A-9C, this effect was most pronounced in the SKBr3 cell line (60% decrease versus 26% decrease in the MCF-7-HER-18 cell line), which has lower levels of baseline LMW cyclin E.

To better determine the effects of decreased HER2-mediated signaling on the G1 checkpoint, the effects were assessed of trastuzumab on cyclin D1, another important G1 regulator, and the CDK inhibitors p21 and p27. Consistent with other reports (Lee et al., 2000) that cyclin D1 expression increases when cells are transfected with HER2/neu, treatment with trastuzumab decreased cyclin D1 levels (FIG. 10B). No appreciable change in p21 or p27 expression (FIG. 10B) was observed; however, confocal immunofluorescence microscopy revealed increased nuclear localization of both CDK inhibitors (FIG. 10D).

FIGS. 10A-10D show the effect of decreased HER2-mediated cell signaling after treatment with trastuzumab. As shown in FIG. 10A, HER2-overexpressing MCF-7-HER-18 and SKBr3 tumor cells were treated with 10 μg/mL or 20 μg/mL of trastuzumab. After 48 hours, confocal immunofluorescence microscopy with antibodies against cyclin E (red) showed a decrease in total cyclin E expression. TO-PRO-3-iodide (blue) was used as a nuclear stain. Cyclin E expression was quantified using Image-Pro Plus Software. As shown in FIG. 10B, to determine the effect of decreased HER2-mediated signaling caused by treatment with trastuzumab on G1 cell cycle regulators, lysates obtained from MCF7, MCF-7-HER-18, and SKBr3 cells that had been treated with trastuzumab were subjected to western blot analysis. Expression of low molecular weight (LMW) cyclin E and cyclin D1 were decreased in cells treated with trastuzumab versus untreated control cells. There was no difference in the expression of the CDK inhibitors p21 or p27 in cells that had been treated with trastuzumab versus untreated control cells. To investigate the effects of decreased HER2-mediated signaling on cyclin E function, standard kinase assays were performed after cells were treated with trastuzumab and harvested and lysates were obtained. Equal amounts of protein (250 μg) were immunoprecipitated with an anti-cyclin E antibody and protein G-sepharose beads using Histone H1 as a substrate (FIG. 10C). For each cell line, bands corresponding to Histone H1 phosphorylation were quantitated through phosphoimaging. As shown, cells treated with trastuzumab had decreased cyclin E-associated kinase compared to untreated control cells in both the MCF-7-HER-18 and SKBr3 cell lines. The decrease in cyclin E-associated kinase activity was greater in the SKBr3 cell line. The MCF7 cell line was used as a HER2 negative control. HER2-overexpressing MCF-7-HER-18 and SKBr3 tumor cells were treated with 10 μg/mL or 20 μg/mL of trastuzumab. After 48 hours, confocal immunofluorescence microscopy with antibodies against p21 (red, left) and p27 (red, right) showed increased nuclear localization of both CDK inhibitors (FIG. 10D). TO-PRO-3-iodide (blue) was used as a nuclear stain.

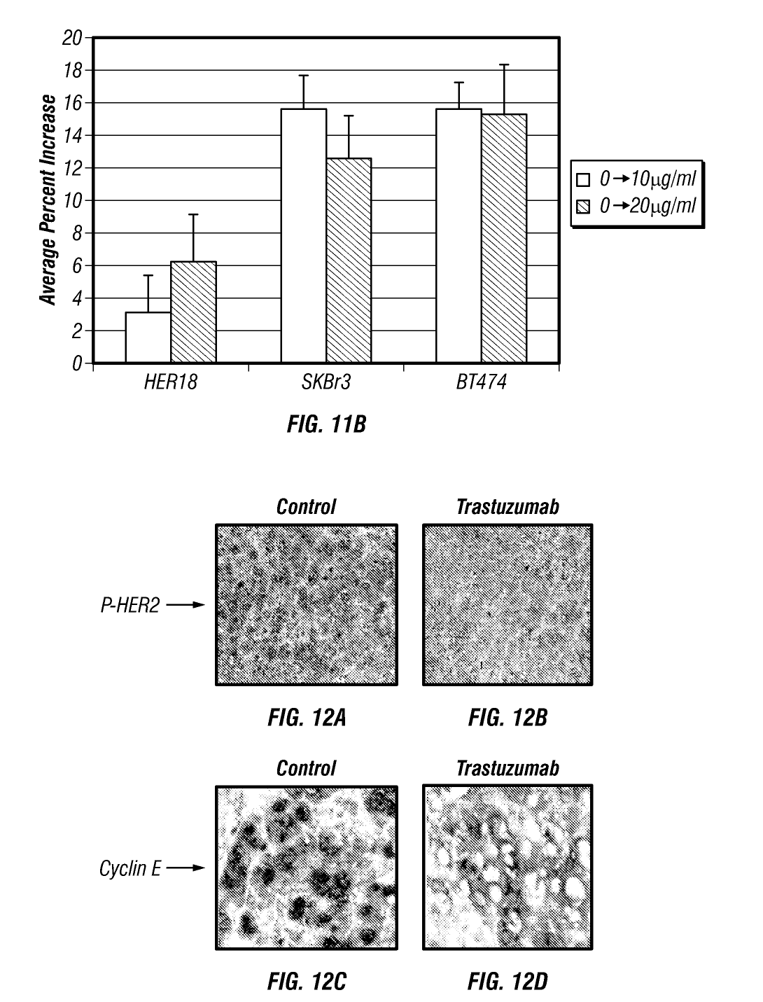

Trastuzumab Therapy Alters Proliferation and Cell Cycle Profiles Because one proposed mechanism of action of trastuzumab is the inhibition of cell proliferation (Bacus et al., 1992; Ben-Bassat et al., 1997; Peng et al., 1996), proliferation was assessed using MTT assays in HER2/neu-overexpressing MCF-7-HER-18 and SKBr3 cells treated with different doses of trastuzumab and found that proliferation decreased in a dose-dependent manner. The number of viable MCF-7-HER-18 cells decreased by 56.8% after treatment with 20 μg/mL of trastuzumab, compared to untreated cells, and the number of viable SKBr3 cells decreased by 67.0% compared to untreated cells (FIG. 11A). Concomitant with the decrease in proliferation was an increased percentage of cells in G1 phase of the cell cycle (FIG. 11B). The mean percentage increase was greater in SKBr3 cells which have endogenous HER2 overexpression than in MCF-7-HER-18 cells which have exogenous HER2 overexpression. These data suggest that breast cancer cells with endogenous HER2 overexpression depend on mitogenic signaling through HER2 pathways to increase cellular proliferation. In contrast, the MCF-7-HER-18 cells that were stably transfected to exogenously express HER2 may have additional oncogenic pathways affecting their rate of cellular proliferation.

FIGS. 11A-B shows alteration in proliferation and cell cycle profiles after treatment with trastuzumab. As shown in FIG. 11A, MCF-7-HER-18 and SKBr3 tumor cells were treated with 10 μg/mL or 20 μg/mL of trastuzumab. Seventy-two hours after treatment, cell number was determined as a measure of proliferation using an MTT assay. Cells treated with trastuzumab had a dose-dependent decrease in the number of viable cells, suggesting that the decreased HER2-mediated signaling caused decreased proliferation. As shown in FIG. 11B, to determine if an alteration in cell cycle distribution contributed to the decrease in proliferation, cell cycle profiles were determined using fluorescence-activated cell sorting (FACS) analysis of MCF-7-HER-18, SKBr3, and BT474 cells that had been treated with trastuzumab. Experiments were repeated in triplicate, and the mean increase in the percentage of cells in the G1 phase of the cell cycle for each dose group, 10 μg/mL and 20 μg/mL, was determined relative to that of untreated controls. There was a statistically significant increase in the percentage increase for both dosing groups of SKBr3 and BT474 cells. The increase in MCF-7-HER-18 cells was not statistically significant.

Inhibition of HER2-Mediated Signaling Results In Decreased Cyclin E Expression In Vivo. To translate the finding that HER2 mediates cyclin E expression in vitro to an in vivo setting, a HER2-overexpressing breast cancer xenograft model was created by injecting MCF-7-HER-18 breast cancer cells into the mammary fat pads of nude mice. After tumors reached 100 mm³, the mice were given intraperitoneal injections of 110 μg of trastuzumab or PBS twice weekly for 3 weeks and then euthanized. Immunohistochemical analyses of the tumors showed that tumors from trastuzumab-treated mice had lower levels of phosphorylated HER2 expression than tumors from control mice, confirming a treatment effect. In addition, there was a concomitant decrease in cyclin E expression in the tumors from trastuzumab-treated mice (FIGS. 12A-12D). These data provide in vivo confirmation of the effects of decreased HER2-mediated signaling on cyclin E expression that was observed in HER2-overexpressing breast cancer cell lines in vitro.

FIGS. 12A-12D show in vivo effects of trastuzumab therapy. MCF-7-HER-18 breast cancer cells were injected into the mammary fat pads of nude mice. When tumors reached 100 mm³, the mice received intraperitoneal injections of either 100 μg of trastuzumab or PBS control twice weekly for 3 weeks. After being euthanized, the tumors were removed from the mice, fixed and then paraffin-embedded. Sections were then stained for phospho-HER2 or cyclin E. Tumors from trastuzumab-treated mice showed decreased phospho-HER2 staining (FIG. 12B versus FIG. 12A), confirming a treatment effect. There was a concomitant decrease in cyclin E expression (FIG. 12D versus FIG. 12C) in the trastuzumab-treated tumors.

### Discussion

This Example demonstrates a novel interaction between HER2 and cyclin E in breast cancer. The downregulation of HER2 using HER2 siRNA and the decrease of HER2-mediated signaling using treatment with trastuzumab both resulted in a decreased expression of cyclin E, particularly the LMW isoforms. This decrease in LMW cyclin E expression decreased cyclin E-associated kinase activity, which in turn decreased proliferation and an increased percentage of cells in the G1 phase of the cell cycle. Taken together, the data disclosed herein provide evidence for a more aggressive phenotype of HER2-overexpressing breast cancers that also overexpress cyclin E. Effective treatment for patients whose tumors overexpress both proteins may thus require targeting HER2 and cyclin E, particularly the LMW-E isoforms.

Even though HER2 is one of the most studied proteins in cancer biology, the exact mechanisms by which HER2 promotes the proliferation of cancer cells is not completely understood. One effect of HER2-mediated signaling is disruption of cell cycle regulation, particularly the G1 checkpoint. These data suggest that the effect of HER2 on the G1 phase may be mediated by its impact on the expression of cyclin E. This finding is important because cyclin E is the predominant regulatory protein at the G1/S transition, and the deregulation of this transition point, which leads to continuous cell proliferation, is a hallmark of cancer.

Numerous reports have demonstrated a linkage between tumorigenesis and cyclin E by correlating the altered expression of cyclin E to the loss of growth control in breast cancer (Keyomarsi and Herliczek, 1997; Buckley et al., 1993; Keyomarsi and Pardee, 1993). One prior report has addressed the effects of trastuzumab therapy on cyclin E. Le et al. demonstrated that treatment of SKBr3 cells with the anti-HER2 antibody 4D5 results in decreased cyclin E-associated kinase activity (Le et al., 2006). These authors did not identify a decrease in cyclin E however they focused on the full-length form. The data herein is consistent with this report in that no appreciable decrease in full-length cyclin E after HER2 downregulation or decreased HER2-mediated signaling was observed. Importantly, on further investigation, a pronounced decrease in the LMW isoforms was observed.

Elastase, a serine protease that cleaves the full-length protein at two sites in the amino terminus (Porter et al., 2001), mediates the generation of these tumor-specific LMW isoforms through the post-translational processing of the full-length cyclin E protein. The qRT-PCR data, which showed no significant change in cyclin E gene expression between HER2 siRNA-transfected cells and control cells, are consistent with these findings. This suggests that the effects of HER2 on cyclin E are post-transcriptional.

Several reports have attributed the effect of HER2 on the G1 phase of die cell cycle to its negative impact on the CDK inhibitors p21 and p27 (Yang et al., 2000; Zhou et al., 2001). The inhibitory potential of p21 has been strongly correlated with its nuclear localization (Chen et al., 1995). For example, Zhou et al. (Zhou et al., 2001) demonstrated that HER2 overexpression induces the cytoplasmic distribution of p21 through the activation of AKT. A second CDK inhibitor that negatively affects the activities of G1 CDKs, p27, is down-regulated by HER2-mediated signaling through the MAPK pathway, with enhanced ubiquitin-mediated degradation (Yang et al., 2000). These data confirm that HER2 affects the localization of p21 and p2′7, as treatment with trastuzumab increased the nuclear localization of both. This finding is particularly important for the subset of HER2-overexpressing tumors that also overexpress LMW cyclin E, in that it has previously been demonstrated that the LMW isoforms are resistant to the growth inhibitory potential of p21 and p27 (Akli et al., 2004; Wingate et al., 2005). Treatment with trastuzumab decreases the extent to which the resistant LMW cyclin E isoforms are expressed and may increase the amount of nuclear p21 and p27 above a threshold level needed to effectively inhibit the activity of the remaining LMW cyclin E.

Data from the current study further demonstrate the existence of a subtype of HER2-overexpressing breast cancer with high levels of LMW cyclin E that is a particularly aggressive phenotype. Based on these data, it is likely that LMW cyclin E will have clinical relevance as a prognostic factor for patients with HER2-overexpressing tumors. Importantly, these findings suggest that tumors overexpressing both HER2 and LMW cyclin E may respond to HER2 targeted-therapy, as HER2 downregulation resulted in decreased cyclin E-associated kinase activity and an increased percentage of cells in G1, which decreased proliferation. Combined with the prognostic role of LMW cyclin E demonstrated by the clinical data in the current study and previous reports (Keyomarsi et al., 2002), this suggests that routinely determining the level of LMW cyclin E in HER2-overexpressing breast tumors may have clinical utility.

In conclusion, an interaction between HER2 and cyclin E has been identified that contributes to the existing knowledge regarding the effects of HER2 overexpression on the regulation of the G1 checkpoint and cellular proliferation. The results show that HER2 acts post-transcriptionally to affect the formation of the tumorigenic LMW isoforms of cyclin E. These data suggest that LMW cyclin E overexpression in HER2-overexpressing breast cancer has both a prognostic and predictive role. In addition, LMW cyclin E may serve as an additional therapeutic target, and further studies investigating the mechanism by which HER2 affects the formation of the LMW forms of cyclin E may lead to the design of new therapeutics.

### Example 3

### Altered Subcellular Localization of LMW-E

Here is presented the an investigation of LMW-E subcellular localization and the effects of LMW-E localization on functional and regulatory protein interactions, and evidence that EL and LMW-E subcellular localization differs, thereby affecting interaction with Cdk2 and proteasomal regulation of complexes formed therefrom.

### Materials and Methods:

Culture conditions: Breast, ovarian, and osteosarcoma cancer cells were grown in Alpha-MEM media (Hyclone, Logan, Utah) supplemented with 10% fetal bovine serum (FBS) (Atlanta Biological, Lawernceville, Ga.). Normal immortalized mammary epithelial cells were grown in D-media (Band et al., 1990) composed of 1/2 HQ Ham's/F-12K (Hyclone) and 1/2 Alpha-MEM media supplemented with 1% FBS. 293T human embryonic kidney cells were grown in D-MEM media (Hyclone) supplemented with 10% FBS. Cells were maintained in a 37° C., 6.5% CO₂ growth chamber.

Plasmids and transfections: To generate cyclin E-IFPN vectors, EL-, T1-, and T2-FLAG sequences were amplified by polymerase chain reaction (PCR) from the cyclin E-FLAG expression vectors (Harwell et al., 2000) and directionally subcloned into the IFPN (VY099) protein complementation vector (Ding et al., 2006) using 5′ HindIII and 3′ ClaI restriction sites. Cdk2 cDNA sequence was PCR amplified and directionally subcloned into the IFPC (VY102) protein complementation vector (Ding et al., 2006) using 5′ ClaI and 3′ HindIII restriction sites. FLAG-Fbw7α and FLAG-Fbw7γ vectors were gifts of the Harper lab (Welcker et al., 2004). Cell transfections were performed using Gene Juice Transfection Reagent (Novagen, Gibbstown, N.J.). Lysates were collected for western blot analysis 72 hours post transfection. To determine transfection efficiency, TRE-luciferase expression vector was co-transfected and luciferase activity measured using the Luciferase Assay II Reagent (Promega, Madison, Wis.). Transfection efficiency was comparable across experiments (data not shown).

In vitro protein generation: Cyclin E (EL, T1, and T2)-IFPN and Cdk2-IFPC fusion proteins were generated in vitro using the TnT Quick Coupled Transcription/Translation Systems (Promega).

Protein isolation: Cells were detached from the plate using a cell scraper and pelleted at 1400 rpm, 4° C., for 5 minutes. Whole cell protein extraction: To extract protein from whole cell lysates, cell pellets were resuspended in NP40 lysis buffer (0.5% NP40, 50 mM Tris, pH 7.5, 150 mM NaCl, 3 mM MgCl₂, and protease inhibitors) and incubated on ice for 1 hour. Cell fractionation: Pelleted cells were resuspended in cold buffer A (20 mM Hepes pH 7.9, 10 mM KCl, 2.5 mM MgCl₂, 1 mM EDTA, 1 mM DTT, 0.2% NP40, and protease inhibitors) and incubated on ice for 5 minutes with occasional pipetting. The cells were then centrifuged at 14000 rpm, 4° C. for 1 minute. The supernatant is the soluble cytoplasmic fraction (C). The pellet was washed twice in buffer A, resuspended in cold buffer B (buffer A, 20% glycerol, 20 mM NaCl), incubated on ice for 30 minutes with occasional pipetting, and centrifuged at 14000 rpm, 4° C. for 20 minutes. The supernatant is the soluble nuclear fraction (N).

Western blot analysis: Western blot analysis was performed as described in Harwell et al., 2000. Western blot membranes were incubated with the following primary antibodies: cyclin E HE-12 (Santa Cruz Biochemicals, Santa Cruz, Calif.), Cdk2 (Transduction Laboratories, Lexington, Ky.), GFP (Abcam, Cambridge, Mass.), grb2 (BD Transduction, San Jose, Calif.), parp (Cell Signaling, Danvers, Mass.), and vinculin (Sigma, St. Louis, Mo.). Western blots were exposed to X-ray film (Phenix Research Products, Chandler, N.C.), and subject to densitometry using ImageQuant TL v2005 software (Amersham Biosciences, Piscataway, N.J.).

Histone HI kinase: Cyclin E associated kinase activity was determined as previously described in Harwell et al. (2000). Briefly, 250 μg of protein was immunoprecipitated with cyclin E polyclonal antibody coupled to Protein A Sepharose beads (GE Healthcare Biosciences AB, Uppsala, Sweden). The immunoprecipitates were then incubated in kinase buffer with 5 μCi [³²P] γ-ATP and 0.33 mg/mL histone H1 and electrophoresed on 10% SDS-PAGE. The gel was stained, destained, exposed to film and subject to densitometry.

Cycloheximide treatment: For cycloheximide (CHX) treatments, growth media was replaced with CHX-treated media (200 or 300 μg/mL CHX). Cells were harvested after 40 and 80 minute incubations with CHX. Control cells (0′) were left untreated.

FACS analysis: The mean fluorescence intensity of intensely fluorescent protein (IFP) was measured for 30,000 cells per biological replicate using fluorescence activated cell sorting (FACS).

Protein complementation and cytoplasmic/nuclear count: Protein complementation: To observe subcellular localization of cyclin E/Cdk2 complexes using protein complementation and microscopy, cells were co-transfected with equal concentrations of cyclin E-IFPN and Cd1(2-IFPC expression vectors and grown on coverslips. Twenty-four to 72 hours post transfection, the cells were fixed in 100% methanol and mounted on slides for microscopy. Nuclei were DAPI stained. Cytoplasmic nuclear counts: To determine the percentage of cells that had nuclear or cytoplasmic/perinuclear IFP fluorescence, fluorescent interphase cells were counted for 5-10 microscopic fields per biological replicate. Cells expressing IFP in both the nucleus and cytoplasm were tallied as a positive for each subcellular compartment.

Microscopy: Images were captured using 10× or 20× objectives on a Leica DM4000B fluorescent microscope (Leica Microsystems, Bannockburn, Ill.) with a RT KE/SE SPOT camera (Diagnostics Instruments, Inc., Sterling Heights, Mich.). The SPOT 4.6 software (Diagnostic Instruments, Inc.) was used to process the images.

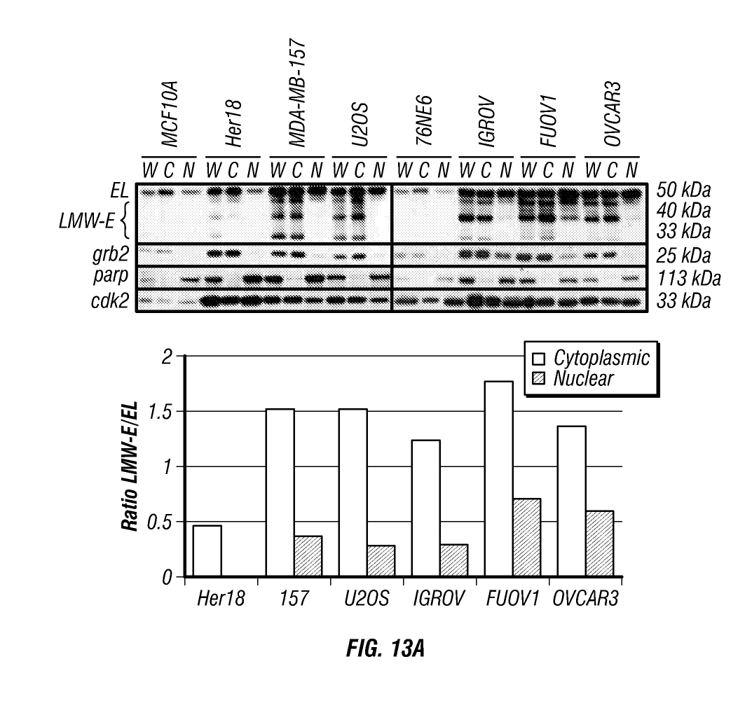

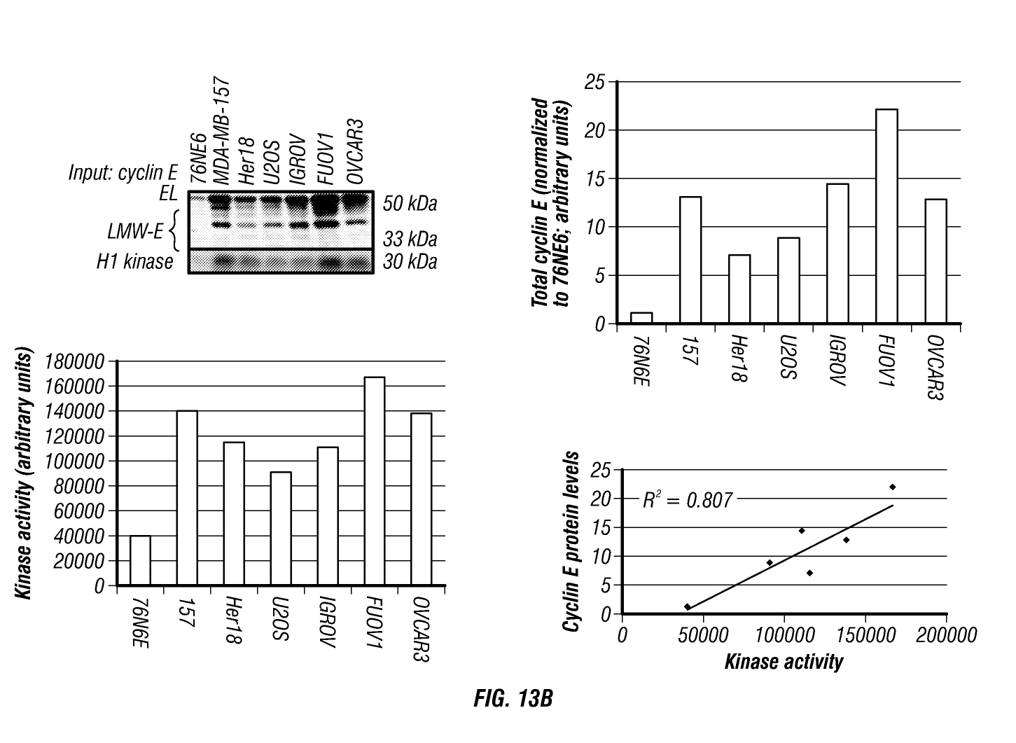

Statistical analysis: Results shown as mean+/− standard deviation were compared by the student's t test with a significant level of p-value (P)<0.05. The nonparametric Spearman correlation was used to quantify the relationship between cyclin E protein expression and kinase activity of FIG. 13B. The P (two-tailed) with a 95% confidence interval was calculated with a significant level of P<0.05.

### Results

### Low Molecular Weight Cyclin E (LMW E) Isoforms Preferentially Accumulate in the Cytoplasm

The LMW-E lack the first 40, 45, or 70 amino acids of EL (Porter et al., 2001). EL contains a canonical nuclear localization sequence (NLS), RSRKRK (SEQ ID NO:6) (Moore et al., 2002), located at amino acids 27-32, which prompted us to investigate LMW-E subcellular localization. Cyclin E distribution was examined in several different cell lines of normal and tumor origin, including two immortalized mammary epithelial cell lines (76NE6 and MCF10A) and in several breast, osteosarcoma and ovarian cancer cell lines that accumulated high levels of LMW-E. The cell lines were separated into whole cell and soluble cytoplasmic and nuclear lysates, which were then subjected to western blot analysis for cyclin E. As expected, the MCF10A and 76NE6 non-tumorigenic cell lines only expressed EL, while tumor cells expressed both EL and LMW-E (FIG. 13A). However, the analysis revealed the novel finding that in all of the cancer types assayed, the LMW-E primarily accumulated in the cytoplasmic fraction (FIG. 13A). Grb2 and Parp expression was used as controls for cytoplasmic and nuclear, respectively, staining.

Densitometry was used to measure LMW-E subcellular accumulation within each subcellular fraction and the ratio of total LMW-E to EL was calculated. In tumor cells, the ratio of total LMW-E to EL in the cytoplasm was consistently greater than the ratio of total LMW-E to EL in the nucleus (FIG. 13A). In fact, LMW-E were only detected in the cytoplasmic fraction of Her18 cells (FIG. 13A). Furthermore, the ratio of total LMW-E to EL in the cytoplasmic fraction of MDA-MB-157, U20S, IGROV, FUOV1, and OVCAR3 cell lines were respectively, 4.2, 5.6, 4.2, 2.5, and 2.3 times greater than that of the LMW-E/EL ratio in the nuclear fraction (FIG. 13A). Thus, LMW-E show a marked subcellular localization to the cytoplasm; and this segregation is specific to tumor cells. Cyclin dependent kinase 2 (Cdk2) is the primary cyclin E co-activator (Johnson and Walker, 1999). I has been previously reported that LMW-E also bind Cdk2 (Akli et al., 2004; Porter et al., 2001; Wingate et al., 2005). Because most LMW-E protein is cytoplasmic, it was desirable to examine the subcellular localization of Cdk2. Western blot analysis showed that Cdk2 protein accumulates in both the cytoplasmic and nuclear fractions (FIG. 13A) suggesting that LMW-E might form complexes with Cdk2 in the cytoplasm.

### Cytoplasmic Cyclin E has Associated Kinase Activity

EL/Cdk2 complexes localize to the nucleus to phosphorylate target proteins (Moroy and Geisen, 2004). However, LMW-E, which are found primarily in the cytoplasm (FIG. 13A), also have associated kinase activity in whole cell extracts (Akli et al., 2004; Harwell et al., 2000; Porter et al., 2001; Wingate et al., 2005). This prompted an investigation of whether cytoplasmic cyclin E has associated kinase activity in tumor cells which accumulate LMW-E.

Cytoplasmic lysates from 76NE6 cells and breast, ovarian, and osteosarcoma cancer cells (FIG. 13B) were immunoprecipitated with cyclin E antibody and assayed the cyclin E immune complexes for kinase activity in vitro using histone H1 as a substrate. Western blot analysis revealed that all tumor cells examined express both EL and LMW in their cytoplasmic fractions, while the 76NE6 normal cells only express the EL (FIG. 13B, top panel). Histone H1 phosphorylation was higher in all tumor cell lines as compared to normal cells (FIG. 13B) suggesting that EL and LMW-E form active kinase complexes in the cytoplasm.

Densitometry was used to quantify the total cyclin E protein levels (EL and LMW-E) and cyclin E associated kinase activity in the non-tumorigenic and tumor cells (FIG. 13B bar graphs). Correlative analysis of these results revealed that there is a positive correlation (R²=0.0807, p-value=0.0060) between cyclin E protein levels and cyclin E associated kinase activity (FIG. 13B, line graph), suggesting that cytoplasmic cyclin E, including the LMW-E, form active kinase complexes in the cytoplasm.

FIGS. 13A-13B show cancer cells accumulate LMW-E in the cytoplasm and have cyclin E associated kinase activity in the cytoplasm. As shown in FIG. 13A, normal mammary epithelial (MCF10A, 76NE6) cell lines and breast (Her18, MDA-MB-157), osteosarcoma (U20S), and ovarian (IGROV, FUOV1, OVCAR) cancer cell lines were separated into whole cell (“W”), cytoplasmic (“C”) and nuclear (“N”) fractions. Whole cell and subcellular fractions were analyzed by western blot probed with antibodies to cyclin E, cd10, grb2, and parp. Grb2 and parp antibodies were used to indicate the cytoplasmic and nuclear fractions, respectively. Western blots were subject to densitometry and the ratio of total LMW-E to full-length cyclin E (EL) was graphed. FIG. 13B shows a western blot of cytoplasmic lysates from normal mammary epithelial cells (76NE6) and cancer cells (MDA-MB-157, Her18, U20S, IGROV, FUOV1, OVCAR3) demonstrating the relative levels of cytoplasmic cyclin E (“Input: cyclin E”) that were assayed for histone H1 phosphorylation (“Hl kinase”). The bar graphs show densitometry values for total cytoplasmic cyclin E (top bar graph; normalized to 76N6E) and HI phosphorylation (bottom bar graph; raw values) for each cell line. The linear regression graph shows correlation between cyclin E levels and cyclin E associated kinase activity for each cell line; R2=0.0807, P=0.0060.

### Protein Complementation Assay as a Tool to Examine Cyclin E/Cdk2 Subcellular Localization and Functionality

Based on the subcellular fraction and kinase data, it was hypothesized that the kinase activity associated with LMW-E in the cytoplasm is through its interaction with CDK2. To directly address this question protein complementation technology was used, which unlike immunoprecipitation and immunoblotting, is not limited by input protein concentration and antibody efficacy. Furthermore, using protein complementation, the formation and localization of cyclin E/Cdk2 interaction can be observed in vivo for each specific cyclin E isoform. Thee intensely fluorescent protein (IFP) complementation method (Ding et al., 2006) was employed. IFP is an improved variant of green fluorescent protein (Nagai et al., 2002) that has been rationally fragmented into N-terminus (IFPN) and C-terminus (IFPC) peptides consisting of amino acids 1 to 158 and 159 to 239, respectively (Remy and Michnick, 2004). When proteins fused to the IFPN and IFPC peptides interact, the IFP fragments are brought into proximity and emit green fluorescence, thereby enabling visualization of the localization of protein-protein interactions in vivo (Remy and Michnick, 2004).

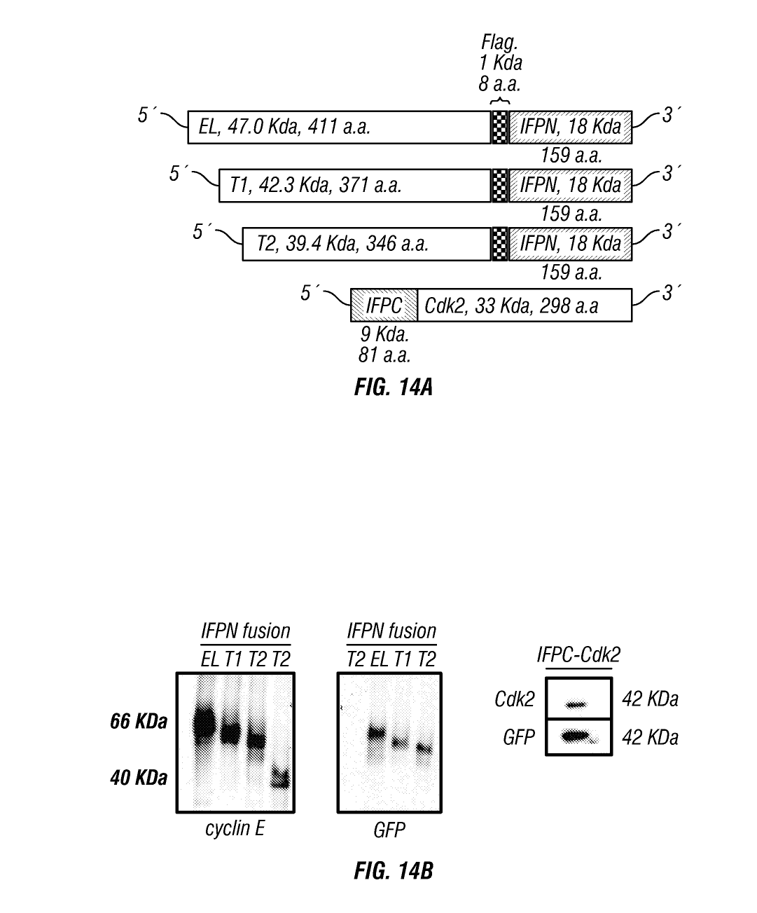

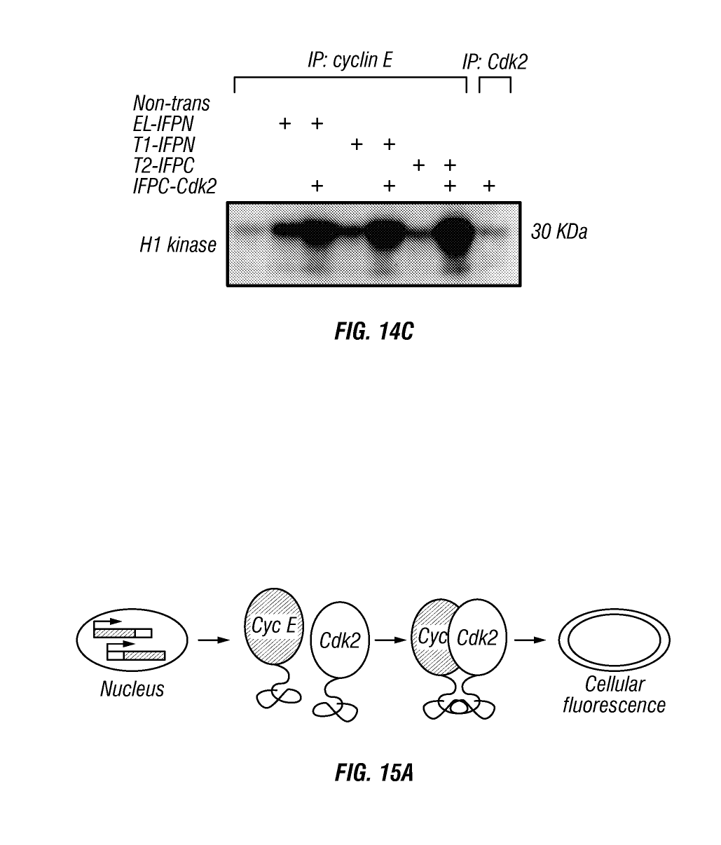

FIG. 14A shows a schematic of the cyclin E and Cdk2 protein complementation constructs used in this study. FLAG-tagged EL cDNA was subcloned and truncated EL sequences representing the elastase-generated LMW-E (termed T1 and T2) into the IFPN expression vector (FIG. 14A). Cdk2 cDNA was subcloned into the complimentary IFPC expression vector.

To determine if the protein complementation constructs could generate fusion proteins, in vitro transcription and translation (TnT) was used. TnT products were analyzed on western blots probed with anti-GFP antibodies, which recognize the IFP fragments, and cyclin E or Cdk2 antibodies. T2-FLAG protein served as a control for mobility and antibody specificity. T2-FLAG is 40.4 kDa and was recognized by the cyclin E antibody but not the by GFP antibody (FIG. 14B). Both the cyclin E and GFP antibodies recognized the EL-, T1-, and T2-IFPN fusion proteins, which migrate at 66, 61.3, and 58.4 kDa, respectively (FIG. 14B). The Cdk2 and GFP antibodies recognized the 42 kDa Cdk2-IFPC fusion protein (FIG. 14B). Thus, cyclin E and Cdk2 IFP fusion proteins were successfully generated.

An in vitro histone HI kinase assay was performed to assess fusion protein functionality using whole cell lysates from 293T human embryonic kidney cells coexpressing cyclin E-IFPN with Cdk2-IFPC. Co-expression with an empty vector and expression of Cdk2-IFPC alone were used as negative controls. The cyclin E-IFPN/Cdk2-IFPC complexes show kinase activity above control background levels (FIG. 14C), indicating that the cyclin E-IFPN and Cdk2-IFPC fusion proteins interact and are functional.

FIGS. 14A-14C show the generation of functional cyclin E and Cdk2 protein complementation fusion proteins. A schematic of the IFP fusion proteins is shown in FIG. 14A. The IFPN fragment was fused to the C-terminus of full length cyclin E (EL-FLAG) and LMW-E (T1-FLAG and T2-FLAG) sequences. The IFPC fragment was fused to the N-terminus of Cdk2. Western blots of in vitro transcription and translation (TnT) cyclin E-IFPN and Cdk2-IFPC fusion proteins were probed with cyclin E, Cdk2, or GFP antibodies as shown in FIG. 14B. As shown in FIG. 14C, in vitro histone H1 phosphorylation was assayed for cyclin E immune complexes immunoprecipitated from 293T whole cell lysates expressing cyclin E-IFPN in the absence or presence of Cdk2-IFPC. Mock-transfected cells or cell transfected with Cdk2-IFPC alone served as negative controls.

### LMW-E Bind to Cdk2 in the Cytoplasm, Perinuclear Membrane, and Nucleus

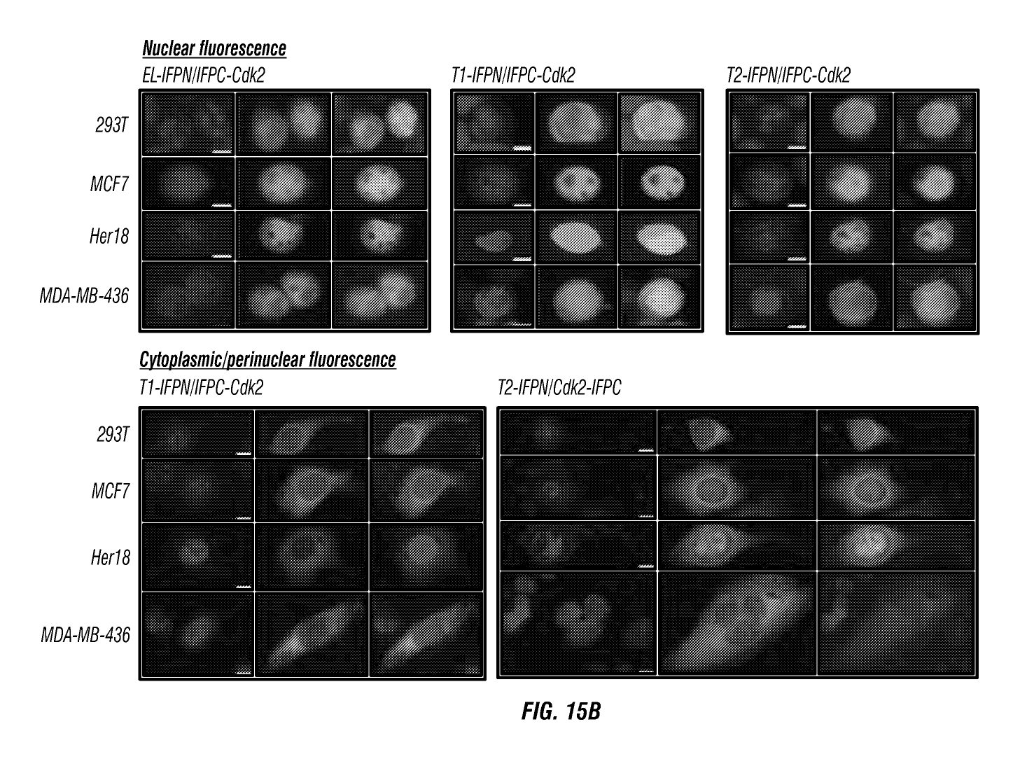

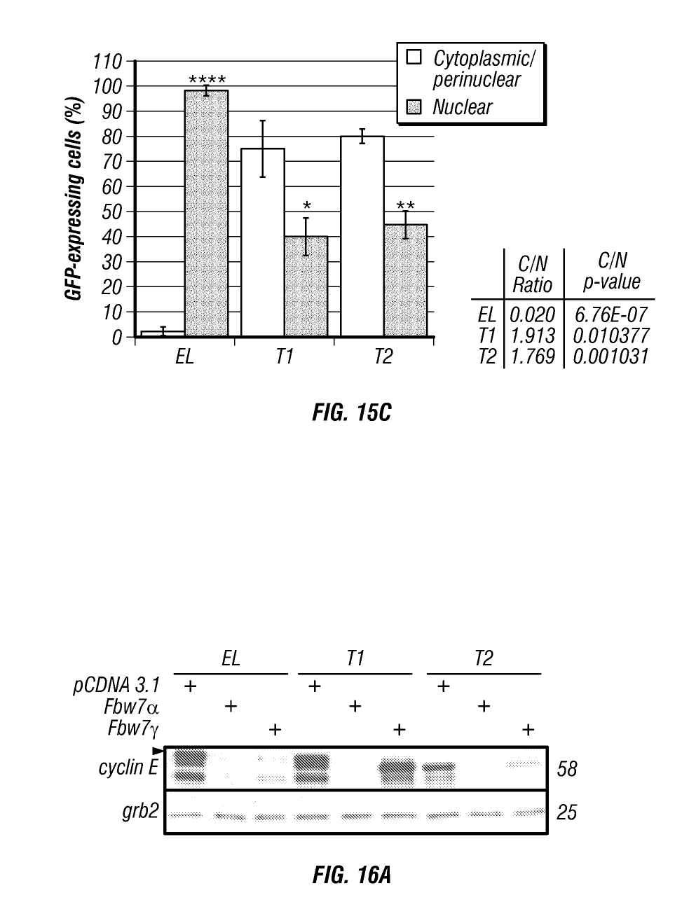

To determine the localization of cyclin E-IFPN/Cdk2-IFPC complexes in vivo the fusion proteins were expressed in mammalian cells and observed IFP green fluorescence using microscopy as depicted (FIG. 15A). The cyclin E-IFPN and Cdk2-IFPC fusion proteins were co-expressed in 293T cells as proof-of-principal and in MCF7, Her18, and MDA-MB-436 breast cancer cells, which are transfectable cell lines that represent a range of physiologically relevant breast cancer cell lines. MCF7 and Her18 (MCF7-derived, Her2 overexpressing) cells are p53, Rb, and ER positive while MDA-MB-436 cells are p53, Rb, and ER negative (Gray-Bablin et al., 1996). In addition, unlike MCF7 cells, Her18 (FIG. 7A) and MDA-MB-436 cells accumulate LMW-E (Koepp et al., 2001).

Upon co-transfection it was observed that in each cell line EL-IFPN and Cdk2-IFPC bound together and emitted green fluorescence at interphase nuclei (FIG. 15B). T1-IFPN/Cdk2-IFPC and T2-IFPN/Cdk2-IFPC complex also localized to interphase nuclei (FIG. 15B). However, unlike EL-IFPN/Cdk2-IFPC, T1- and T2-IFPN/Cdk2-IFPC complexes were also readily detectable in the cytoplasm and at the perinuclear membrane of interphase cells (FIG. 15B).

Co-transfection of IFP empty vectors, or transfection of cyclin E-IFPN or Cdk2-IFPC alone, served as negative controls. The negative controls did not have IFP signal, indicating that the fluorescence observed in cyclin E-IFPN/Cdk2-IFPC expressing cells was due specifically to cyclin E/Cdk2 interaction. Thus the protein complementation results imply that the endogenous cytoplasmic LMW-E that is detected by western blot may indeed form complexes with Cdk2 in the cytoplasm.

Given LMW-E accumulate in the cytoplasm (FIG. 13A) it was interesting to consider whether the T1- or T2-IFPN/Cdk2-IFPC complexes might also favor cytoplasmic localization. Therefore, the percentage was calculated of interphase cells emitting IFP fluorescence in the cytoplasm and/or perinuclear membrane versus those cells emitting fluorescence in the nucleus to determine the ratio of cytoplasmic/perinuclear to nuclear subcellular localization. These experiments were performed using 293T cells because of their high transfection efficiency. Fluorescent EL-IFPN/Cdk2-IFPC expressing cells showed IFP signal almost exclusively in the nucleus of interphase cells (FIG. 15C, P<0.0001). Ti- and T2-IFPN/Cdk2 expressing cells, on the other hand, emitted fluorescence nearly twice as much in the cytoplasm/perinuclear membrane than in the nucleus (FIG. 15C, P<0.05 and p<0.01). Thus, while EL-IFPN/Cdk2-IFPC complexes preferentially localize to the nucleus, T1- and T2-IFPN/Cdk2-IFPC complexes preferentially localize to the cytoplasm and/or perinuclear membrane. These protein complementation results correlate with endogenous LMW-E subcellular distribution and suggest that LMW-E localization affects the formation and localization of LMW-E protein interactions. To investigate whether LMW-E subcellular distribution might also affect its regulation, EL and LMW-E proteasomal degradation were compared.

### LMW-E are Susceptible to Proteasomal Degradation

Cyclin E proteasomal degradation is regulated, in part, by its subcellular localization and interaction with Cdk2. For example, cyclin E/Cdk2 complexes reside in the nucleus (Jackman et al., 2002) where they are targeted for ubiquitination by the nuclear isoforms of the E3 ubiquitin ligase, Fbw7 (van Drogen et al., 2006; Ye et al., 2003). Alternatively, the Cu13 E3 ubiquitin ligase, which is predominantly localized to the cytoplasm (Watai et al., 2007), targets free cyclin E (not bound to Cdk2) (Singer et al., 1999).

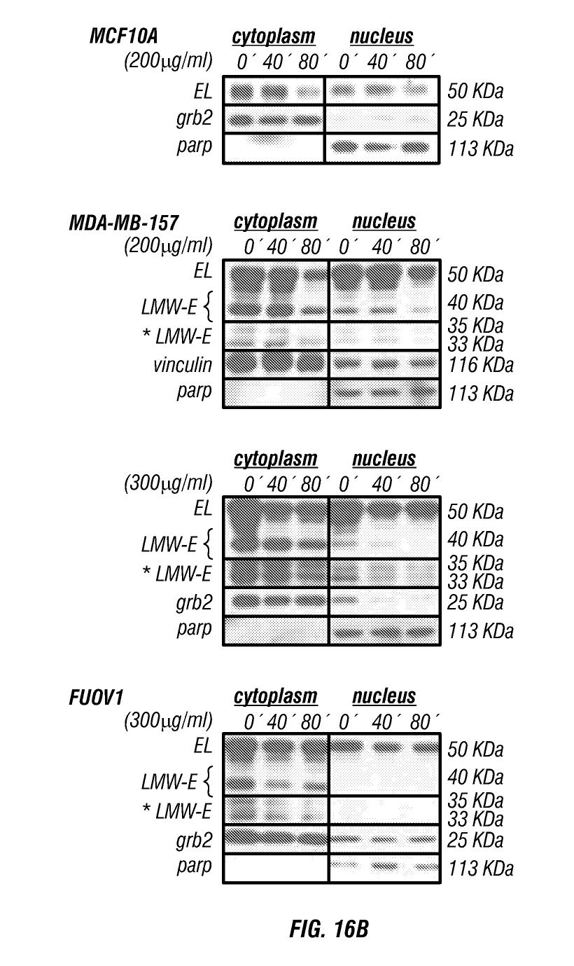

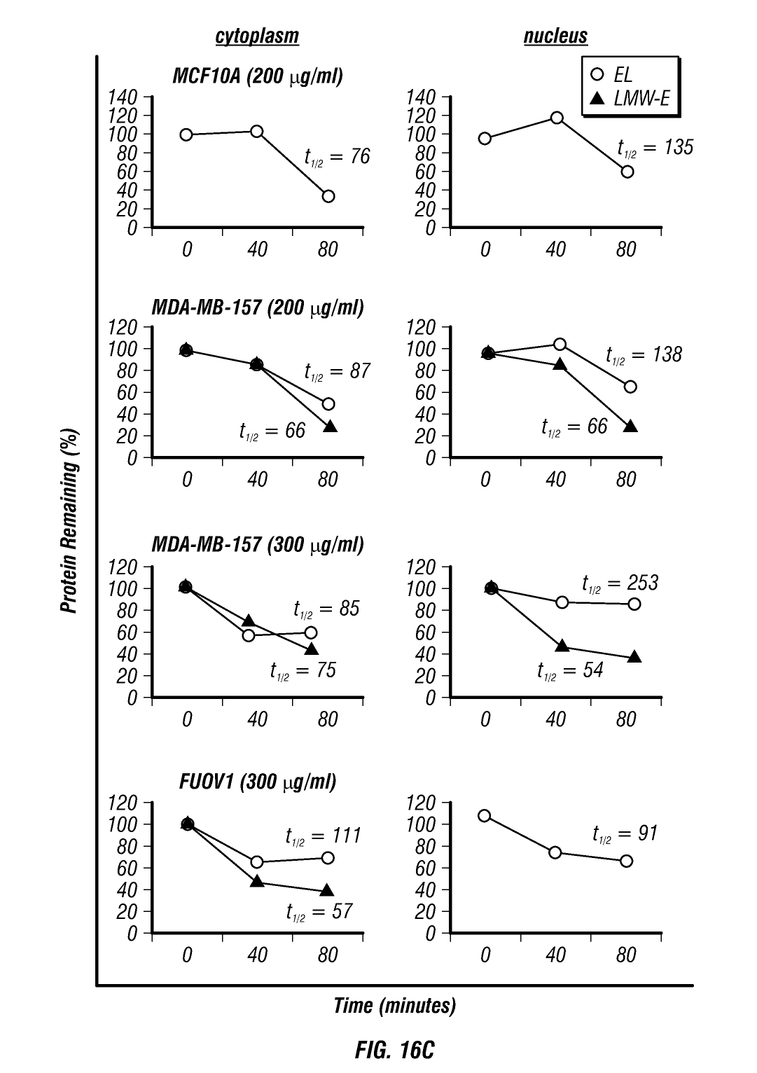

Fbw7-mediated proteasomal degradation of cyclin E requires that Cdk2 bind and phosphorylate cyclin E (Welcker et al., 2003). Given that cyclin E/Cdk2 resides in the nucleus (FIG. 15B; (Jackman et al., 2002), it is not surprising that the nuclear Fbw7α and γ isoforms target cyclin E, while cytoplasmic Fbw7β is not required for cyclin E degradation (van Drogen et al., 2006). However, since the results suggest that in cancer cells LMW-E/Cdk2 favors non-nuclear accumulation (FIG. 15C) it was reasonably questioned whether LMW-E would be susceptible to Fbw7-mediated degradation. To begin to address this question the effect of Fbw7α and γ on T1- and T2-IFPN protein levels was analyzed by cotransfecting MDA-MB-436 breast cancer cells with EL-, T1-, or T2-IFPN and Cdk2-IFPC in the absence or presence of Fbw7α or γ expression vectors. Cells were collected 48 hours after transfection and whole cell lysates analyzed by western blot analysis with cyclin E. Expression vector transfection efficiency was comparable as measured by luciferase activity (see Materials and Methods). The results revealed that EL-, T1- and T2-IFPN protein levels were significantly reduced in the presence of Fbw7 (FIG. 16A), suggesting that Fbw7 does in fact regulate both EL and LMW-E protein stability.

To confirm the proteasomal regulation of endogenous LMW-E, cells were treated with cyclohexamide (CHX) which blocks new protein synthesis thereby allowing us to determine the degradation rate of existing cyclin E protein. To directly compare the stability of each cyclin E isoform, MDA-MB-157 breast and FUOV1 ovarian cancer cell lines were used. MCF10A normal immortalized cells served as a non-tumorigenic control for experimental efficacy. These three different cell lines were treated with different, but equitoxic doses of CHX as there is normal/tumor differential sensitivity to CHX. Following treatment of cells with CHX, cytoplasmic and nuclear lysates were isolated and analyzed by western blot for cyclin E (FIG. 16B) and quantified using densitometry (FIG. 16C). The results showed that EL levels were reduced in the CHX-treated non-tumorigenic and cancer cells (FIGS. 16B-16C). LMW-E levels also declined in the CHX-treated cancer cells and appeared slightly less stable than EL (FIGS. 16B-16C). Thus, the data indicate that like EL, endogenous LMW-E are susceptible to degradation by the proteasome in cancer cells.

Cytoplasmic LMW-E/Cdk2 Complexes have Reduced Sensitivity to Fbw7-mediated Degradation

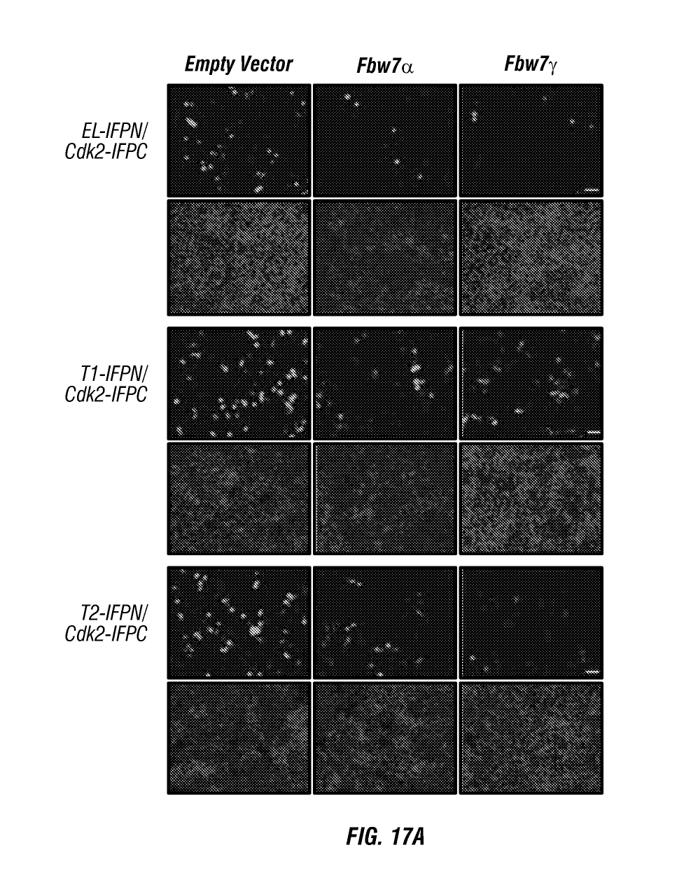

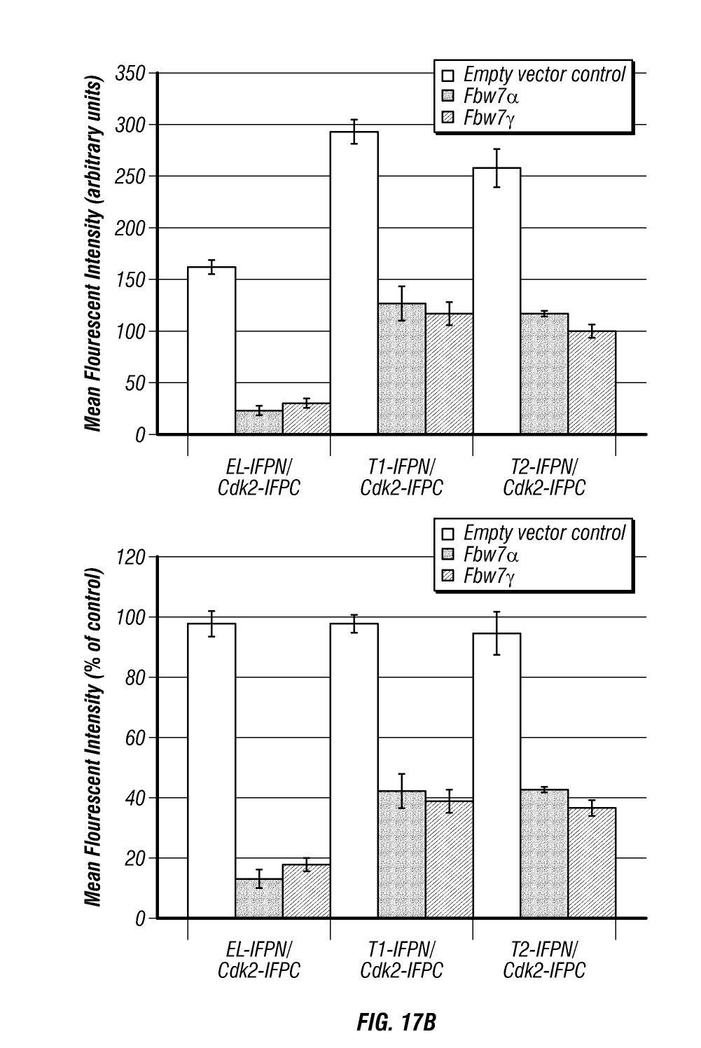

While LMW-E (i.e. T1 and T2) are sensitive to Fbw7-mediated proteasomal degradation, it was not known how Fbw7-mediated degradation affects subcellular distribution of LMW-E/Cdk2 complexes. This is an important question because LMW-E tumorigenic potential is not expected to exclusively correlate with its protein levels, but other properties as well, such as its spatial and temporal localization and biochemical interaction with other proteins. To determine the effect of Fbw7-mediated degradation on cyclin E, the protein complementation assay was used to examine the effect of Fbw7 on cyclin E-IFPN/Cdk2-IFPC subcellular distribution using fluorescence microscopy. Cyclin E-IFPN (EL, T1, or T2) and Cdk2-IFPC were co-expressed in 293T cells in the absence or presence of Fbw7α or γ and analyzed the cells 24 hours after transfection. Representative images of the co-transfected cells are shown in FIG. 17A. In the absence of Fbw7, cyclin E and Cdk2 IFP fusion proteins formed numerous and intense fluorescent complexes (FIG. 17A, “empty vector”). In the presence of Fbw7α or γ, cyclin E-IFPN/Cdk2-IFPC fluorescent signal was significantly reduced (FIG. 17A).

Fluorescence activated cell sorting (FACS) was then used to quantify cyclin E-IFPN/Cdk2-IFPC IFP signal in the absence or presence of Fbw7 (FIG. 17B). The mean fluorescence (FIG. 17B, top graph) and the normalized percent fluorescence (FIG. 17B, bottom graph) were quantified. The results revealed that EL-IFPN/Cdk2-IFPC signal was reduced by 80% and T1- and T2-IFPC/Cdk2 signal were reduced by 60% in the presence of Fbw7 (FIG. 17B, bottom graph). Ti- and T2-IFPC/Cdk2 mean fluorescence in the control cells was higher than EL-IFPN/Cdk2-IFPC (FIG. 17B, top graph), which may explain why Fbw7 had less effect on T1- and T2-associated IFP signal than that of EL. Nevertheless, these results indicate that Fbw7 can reduce cyclin E/Cdk2 formation.

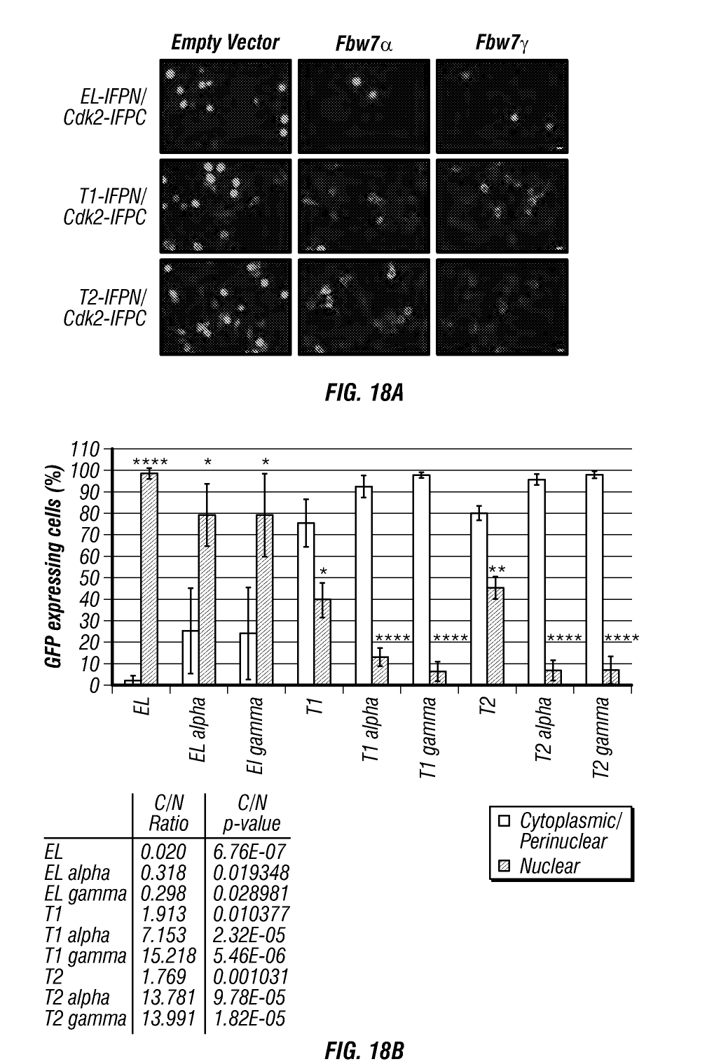

To address the effect of Fbw7 on cyclin E-IFPN/Cdk2-IFPC subcellular distribution the localization of these complexes in vivo was examined using microscopy. A striking trend was discovered involving cyclin E-IFPN/Cdk2-IFPN subcellular accumulation in the presence of Fbw7. Specifically, Fbw7 interfered with nuclear cyclin E-IFPN/Cdk2-IFPC complex formation significantly more than cytoplasmic and/or perinuclear complex formation (FIG. 18A). To quantify this observation, the percentage was determined of cells expressing IFP in the cytoplasm/perinuclear membrane versus nucleus in the absence or presence of Fbw7 (FIG. 18B, bar graph) and used the calculations to determine the ratio of cytoplasmic/perinuclear to nuclear IFP expression (FIG. 18B, table). The total percentages exceeded 100% because the cells that showed signal in both the cytoplasm/perinuclear membrane and in the nucleus were included in both subcellular calculations.

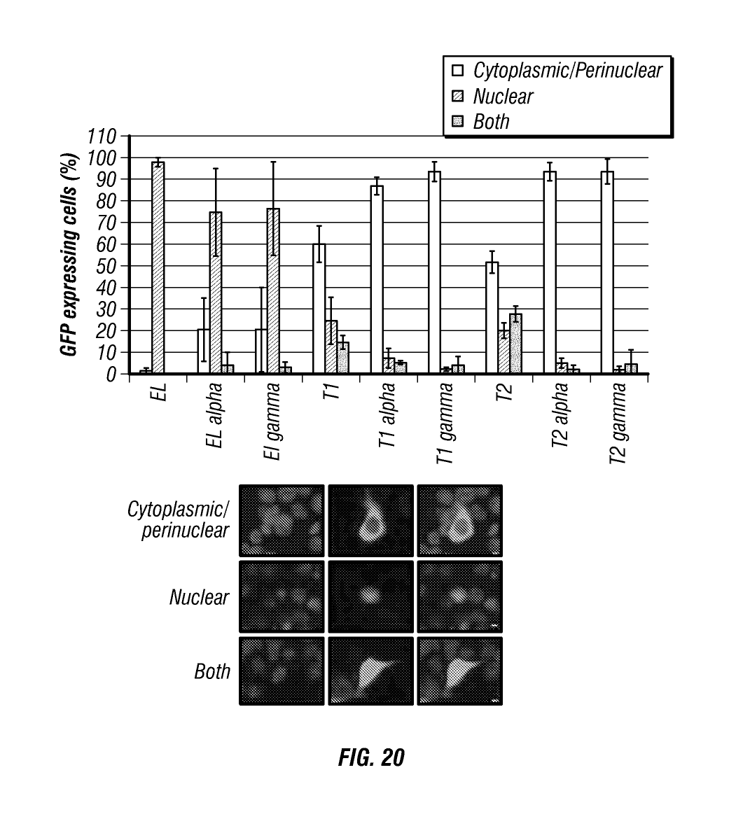

A graph showing the breakdown of cells expressing IFP signal in the cytoplasmic/perinuclear membrane, the nucleus, or in both subcellular compartments is shown in FIG. 20. The ratio of cytoplasmic/perinuclear IFP expression to nuclear IFP expression was greater in the presence of Fbw7 than in the absence of Fbw7 for EL-, Ti-, and T2-IFPN/Cdk2-IFPC (FIG. 18B, table). In particular, the subcellular distribution of Ti- and T2-IFPN/Cdk2-IFPC complexes was significantly more stratified in the presence than in the absence of Fbw7 (FIG. 18B, P<0.0001). Taken together, these data suggest that tumor cells that accumulate LMW-E have acquired a growth advantage due to LMW-E stability because the physiologically relevant nuclear Fbw7 isoforms appear to preferentially target nuclear cyclin E/Cdk2 complexes and in fact, change the subcellular distribution of cyclin E/Cdk2 complexes to favor cytoplasmic accumulation.

### Discussion

LMW-E have greater tumorigenicity than full-length cyclin E (EL) (Akli et al., 2004; Bedrosian et al., 2004; Corin et al., 2006; Porter et al., 2001; Wingate et al., 2003; Wingate et al., 2005) and have been correlated with decreased survival and poor response to therapy in patients (Davidson et al., 2007; Keyomarsi et al., 2002). Therefore, it is imperative to determine the factors responsible for the increased tumorigenic potential of LMW-E in order to develop effective treatment strategies for patients whose tumors accumulate these isoforms of cyclin E. These findings suggest that the loss of the N-terminus affects LMW-E subcellular localization, the localization of cyclin E binding proteins, and LMW-E regulation by the proteasome. Thus, deregulated localization of cyclin E through the generation of LMW may be a mechanism by which cancer cells alter cyclin E regulation and function, imparting a growth advantage over normal cells.

The subcellular localization of full-length cyclin E and the cyclin E/Cdk2 complexes is conserved in both non-tumorigenic cells and cancer cells. Reports have previously shown that full length cyclin E is localized to both the cytoplasm and the nucleus, and that this subcellular distribution pattern is similar in both non-tumorigenic cells and cancer cells (FIG. 13A). In normal cells, full-length cyclin E forms an active complex with Cdk2 that is localized to the nucleus to target nuclear substrates such as pRb, NPAT, and Cd6C. Through these complexes, cyclin E mediates critical nuclear processes including gene transcription and DNA replication (Furstenthal et al., 2001; Moroy and Geisen, 2004; Zhao et al., 2000). Likewise, in tumor cells full-length cyclin E binds Cdk2 almost exclusively in the nucleus where it is also able to influence transcription and DNA replication (FIGS. 15B-15C). Thus, cancer cells maintain the subcellular distribution of full-length cyclin E and its complexes with Cdk2 making it susceptible to the same regulation and rate of degradation and cellular turnover that is seen in normal cells.

One notable difference between normal cells and cancer cells is the generation of the LMW-E isoforms of the cyclin E protein (Porter et al., 2001). The LMW-E protein sequence is identical to full-length cyclin E with the exception of the first 40-70 N-terminal amino acids that have been eliminated from the LMW-E (Porter et al., 2001). It has been previously shown that LMW-E binds to Cdk2 and the cyclin kinase inhibitors (Cki), p21 and p27, with greater affinity than full-length cyclin E and has increased kinase activity compared with full-length cyclin E. However, LMW-E, have reduced sensitivity to Cki proteins (Akli et al., 2004; Bedrosian et al., 2004; Harwell et al., 2000; Porter et al., 2001; Wingate et al., 2005), indicating that the N-terminus is necessary for proper regulation of cyclin E activity within the cell.

Structural analysis suggests that N-terminal truncation could alter LMW-E conformation and subsequent Cdk2 binding (Honda et al., 2005). N-terminal truncation also removes a canonical NLS motif (REF), which may contribute to the aberrant sub-cellular localization patterns observed for LMW-E and the LMW-E/Cdk2 complexes in cancer cells. Specifically, LMW-E and LMW-E/Cdk2 complexes preferentially accumulate in the cytoplasm (FIG. 13A and FIGS. 15A-15C) although it is important to note that LMW-E and LMW-E/Cdk2 complexes are also found in the nucleus of cancer cells (FIG. 13A and FIGS. 15A-15C). Previous studies have shown that the NLS motif of cyclin E is not required for cyclin E nuclear localization (Geisen and Moroy, 2002; Kelly et al., 1998; Porter et al., 2001). Therefore, altered LMW-E and LMW-E/Cdk2 sub-cellular localization may be a mechanism through which cancer cells deregulate the activity of cyclin E.

Cyclin E and cyclin E/Cdk2 subcellular localization is not a static process and, in fact, cyclin E/Cdk2 complexes are known to shuttle between the cytoplasm and nucleus (Jackman et al., 2002). However, cyclin E/Cdk2 nuclear import is believed to be faster than its export, leading to detection of cyclin E/Cdk2 accumulation primarily in the nucleus (Jackman et al., 2002). Given that LMW-E/Cdk2 complexes are found accumulating in both the cytoplasm and nucleus (FIGS. 15B-15C), indicates that there are differences in cyclin E and LMW-E nucleocytoplasmic shuttling dynamics (FIGS. 19A-19B). It appears that LMW-E/Cdk2 may be imported into the nucleus at a slower rate than cyclin E/Cdk2 likely due to the fact that LMW-E do not have the N-terminal NLS motif to bind importin a/13 (Moore et al., 2002). Likewise, an exportin-independent mechanism used to transport cyclin E back into the cytoplasm (Jackman et al., 2002) may be more effective for LMW-E translocation. Once the process of cytoplasmic translocation of the LMW-E is facilitated in tumor cells, these forms of cyclin E will have altered function, since now they are no longer limited to the nuclear environment. Further investigating the nucleocytoplasmic shuttling dynamics for cyclin E, LMW-E, and their protein complexes in cancer cells will be important in unraveling this process.

Spatio-temporal differences in cyclin E/Cdk2 and LMW-E/Cdk2 complexes may effect the proteasomal regulation of these protein complexes in cancer cells. It was hypothesized that the nuclear localized Fbw7 isoforms, which have been shown to be responsible for cyclin E degradation (van Drogen et al., 2006; Ye et al., 2004), only interact with nuclear cyclin E/Cdk2 complexes (FIGS. 19A-19B, model). Given that LMW-E and LMW-E/Cdk2 complexes preferentially localize to the cytoplasm, it was expected that LMW-E would have reduced sensitivity to the proteasome. To the contrary, LMW-E appear to be more sensitive to proteasomal degradation when compared with full-length cyclin E (FIG. 16C). Fbw7-mediated degradation of cyclin E requires cyclin E/Cdk2 autophosphorylation (Welcker et al., 2003), which can be blocked by the Cki proteins p21 and p27. Thus, one possibility is that the increased associated kinase activity and the resistance of LMW-E to inhibition by Cki proteins (Akli et al., 2004; Bedrosian et al., 2004; Harwell et al., 2000; Porter et al., 2001; Wingate et al., 2005) may render them more sensitive to proteasomal degradation.

Interestingly, upon close inspection of residual cyclin E/Cdk2 complexes following Fbw7 treatment, it was observed that despite reduction in both cytoplasmic and nuclear cyclin E/Cdk2 complex formation (FIGS. 17A and 18A), the subcellular distribution of the remaining cyclin E/Cdk2 complexes shifted towards localization to the cytoplasm (FIGS. 18A-18B). This effect was most striking for the LMW-E/Cdk2 complexes which preferentially accumulate in the cytoplasm in the absence of Fbw7 (FIG. 15C). This supports the model that Fbw7 targets the nuclear cyclin E/Cdk2 complexes (FIGS. 19A-19B). Finally, its is likely that the reason a net loss in protein is observed in both subcellular fractions (FIGS. 16B, 16C, and 18A) may be due to cyclin E/Cdk2 complexes shuttling between nuclear and cytoplasmic compartments (FIGS. 19A-19B; Jackman et al., 2002). It is highly likely that the LMW-E subcellular accumulation and nucleocytoplasmic shutting dynamics also effects LMW-E regulation by other cyclin E regulatory proteins.

In addition to regulation of FBw-7 mediated cellular turnover, the differences in the localization of full-length cyclin E and LMW-E and their Cdk2 complexes may also affect LMW-E function (i.e. associated kinase activity) in cancer cells. It was observed here that cyclin E associated kinase activity is present in the cytoplasm of cancer cells (FIG. 13B). Given that LMW-E have a much higher associated kinase activity than full-length cyclin E (Akli et al., 2004; Harwell et al., 2000; Porter et al., 2001; Wingate et al., 2005), it is likely that LMW-E contribute significantly to the observed cytoplasmic kinase activity. Additionally, given that LMW-E have altered biochemical interactions with cyclin E binding proteins such as Cdk2, p21, and p27 (Akli et al., 2004; Harwell et al., 2000; Porter et al., 2001; Wingate et al., 2005), LMW-E/Cdk2 complexes that accumulate in the cytoplasm may have other altered cyclin E binding protein interactions, as well as alternate substrates, that contribute to LMW-E-induced tumorigenicity.

Immunohistochemistry studies have shown that cyclin E is detected primarily in the nucleus of cancer cells (Donnellan and Chetty, 1999). Although cyclin E staining in the cytoplasm has been reported, it has been deemed biologically insignificant because the canonical cyclin E functions and consequences of cyclin E deregulation are known to affect nuclear G1/S events (Donnellan and Chetty, 1999). However, recent reports show that cyclin E does localize to centrosomes to induce centrosome duplication (Matsumoto and Mailer, 2004; Nishimura et al., 2005), and thus cyclin E has non-nuclear functions that have not previously been a focus of its activities in cells. Furthermore, expression of cytoplasmically localized cyclin E constructs was shown to delay mitotic exit in Xenopus oocytes (Moore et al., 2002) and transformed primary rat embryo fibroblasts demonstrating activities that extend well beyond its regulation of the G1/S transition of the cell cycle (Geisen and Moroy, 2002). Thus, cyclin E immunostaining detected within the cytoplasm may in fact signify genomic instability, a hallmark of cancer cells.

Indeed, genomic instability has been correlated with cyclin E overexpression in a breast cancer cell line (Willmarth et al., 2004) and in tissue samples from lung cancer patients (Koutsami et al., 2006), both of which showed LMW-E proteins by western blot analysis in addition to accumulaion of cyclin E protein in the cytoplasm To this end, it may be propose that cytoplasmic cyclin E accumulation observed in tumors is physiologically relevant and may in fact indicate the presence of the tumorigenic LMW-E.

In summary, the LMW-E are tumor-specific proteins that are known to have higher kinase activity than their full-length counterparts and result in decreased survival and poor response to therapy when detected in the tumors of cancer patients. Their function and regulation require further study in order to understand the mechanisms of cell transformation which can then be exploited for design of therapies specific to cancer cells without injury to normal cells. The above disclosed experiments have shown that full-length cyclin E and LMW-E are differentially regulated at the level of subcellular accumulation in cancer cells. Altered LMW-E and LMW-E/Cdk2 complex localization appears to affect LMW-E regulation by cellular regulatory proteins and has implications for LMW-E function. Thus, it is a worthy goal to further uncover differences in the mechanisms of full-length cyclin E and LMW-E regulation and function, so that it is possible to design novel treatment strategies for patients that accumulate the tumorigenic LMW-E.

As shown in FIGS. 15A-15C, protein complementation revealed that LMW-E/Cdk2 complexes localize to the cytoplasm. A schematic of IFP protein complementation is shown (FIG. 15A). Cyclin E-IFPN and Cdk2-IFPC expression vectors are co-expressed in mammalian cells. When cyclin E and Cdk2 bind, the IFPN and IFPC fragments are brought into proximity and emit green fluorescence. Cyclin E (EL, T1, or T2)—IFPN and Cdk2-IFPC expression vectors were co-expressed in 293T, MCF7, Her18, and MDA-MB-436 cells (FIG. 15B). Cells expressing nuclear (top panel) or cytoplasmic/perinuclear (bottom panel) green fluorescence are shown. Images were taken with the 20× objective. Scale bar=5 μm. As shown in FIG. 15C, the table shows the ratio of cytoplasmic/perinuclear (C) to nuclear (N) green fluorescent 293T cells that accumulate cyclin E-IFPN/Cdk2-IFPC complexes. Cells that showed ubiquitous green fluorescent were counted towards both the cytoplasmic/perinuclear and nuclear tallies. A total of 606≦n≦767 green fluorescent cells over 5-10 microscopic fields were counted for each condition. The standard deviation was calculated for three biological replicates. Student t-test was performed for cytoplasmic/perinuclear versus nuclear localization for each condition; P<0.05.

FIGS. 16A-16C show that LMW-E are sensitive to proteasomal degradation. MDA-MB-436 breast cancer cells were transfected with EL-, T1-, or T2-IFPN in the absence (pcDNA3.1 empty vector control) or presence of Fbw7α or γ (FIG. 16A). Whole cell lysates were subjected to western blot analysis for cyclin E. Grb2 is a loading control. MCF10A normal immortalized mammary epithelial cells and MDA-MB-157 breast and FUOV1 ovarian cancer cell lines were treated with 200 and/or 300 μg/ml cyclohexamide for 40 or 80 minutes. Control cells were left untreated (“0” minutes; FIG. 16B). Cells were separated into cytoplasmic and nuclear fractions and analyzed by western blots probed with cyclin E antibody. * indicates a darker film exposure. Western blot densitometry values for cyclin E were used to plot the percent degradation for full length (EL) and total LMW-E cyclin E (FIG. 16C). The 0 minute controls for EL and LMW-E for each experiment was set at 100%.

FIGS. 17A-17B shows that Fbw7 reduces cyclin E-IFPN/Cdk2-IFPC complex formation: As shown in FIG. 17A, 293T cells were co-transfected with EL-, T1-, or T2-IFPN and Cdk2-IFPC in the absence (pcDNA3.1 empty vector) or presence of Fbw7α or γ. Twenty-four hours after transfection, cells were fixed for microscopy. Images were taken at 10× magnification. Green fluorescent cyclin E-IFPN/Cdk2-IFPC expressing cells are shown in the top panel. DAPI stained nuclei (bottom panel) are shown to demonstrate that each field has a comparable number of cells. FACS analysis was used to measure IFP fluorescence in co-transfected 293T cells (FIG. 17B). Top graph shows mean fluorescent intensity and the bottom graph is normalized to the empty vector control. Error bars represent standard deviation of three biological replicates.

FIGS. 18A-18B shows that cytoplasmic cyclin E-IFPN/Cdk2-IFPC localization render the LMW-E isoforms less susceptible to Fbw7-mediated degradation. FIG. 18A shows the localization of cyclin E-IFPN/Cdk2-IFPC complexes in 293T cells co-transfected with EL-, T1-, or T2-IFPN and Cdk2-IFPC in the absence (pcDNA3.1 empty vector) or presence of Fbw7α or γ. The bar graph in FIG. 18B shows the percentage of green fluorescent 293T cells that show cyclin E-IFPN/Cdk2-IFPC signal in the cytoplasm/perinuclear membrane or the nucleus. Cells that showed ubiquitous green fluorescent were counted towards both the cytoplasmic/perinuclear and nuclear tallies. A total of 386≦n≦767 green fluorescent cells over 5-10 microscopic fields were counted for each condition. Error bars represent the standard deviation of three biological replicates; P<0.05 (*), P<0.01 (**), P<0.001 (***), and P<0.0001 (****). The table shows the ratio of cytoplasmic/perinuclear versus nuclear IFP signal that was calculated using the mean percentages for each condition.

A proposed model of the effect of subcellular localization on cyclin E stability and activity in normal versus tumor cells is shown in FIGS. 19A-19B. Based on the literature and these data, it can be proposed that in normal cells, full-length cyclin E (EL) shuttles between the nucleus and cytoplasm (black arrows; FIG. 19A). Nuclear import is faster than export causing autophosphorylated EL/Cdk2 complexes to accumulate in the nucleus where nuclear Fbw7 can target EL for degradation. EL shuttling results in a net loss of protein from both subcellular pools that can be observed in the presence of Fbw7 or CHX treatments (FIG. 19B) EL (light blue circle): Based on this data it is likely that in tumors, EL shows similar subcellular distribution patterns and targeting by Fbw7 as outlined for normal cells. However, EL proteins may be more stable in tumor cells because they accumulate at higher levels than in normal cells. LMW-E (dark blue circle): LMW-E and LMW-E/Cdk2 complexes preferentially accumulate in the cytoplasm possibly due to lack of the NLS and altered shuttling dynamics (gray arrows). Nuclear Fbw7 targets nuclear autophosphorylated LMW-E/Cdk2 complexes, however residual LMW-E/Cdk2 complexes localize to the cytoplasm where they are resistant to Fbw7. LMW-E shuttling results in a net loss of protein from both subcellular pools that can be observed in the presence of Fbw7 or CHX treatments. Finally EL/Cdk2 and LMW-E/Cdk2 complexes have higher cytoplasmic activity in tumor cells as compared to normal cells, which do not accumulate EL and/or generate LMW-E.

Cytoplasmic cyclin E-IFPN/Cdk2-IFPC localization may render the LMW-E isoforms less susceptible to Fbw7-mediated degradation (FIG. 20). The bar graph shows the percentage of green fluorescent 293T cells that show cyclin E-IFPN/Cdk2-IFPC signal in the cytoplasm/perinuclear membrane, the nucleus, or in both subcellular compartments. A total of 386<n<767 green fluorescent cells over 5-10 microscopic fields were counted for each condition. Error bars represent the standard deviation of three biological replicates. The images shown represent cells expressing IFP in the cytoplasm/perinuclear membrane, nucleus, or both subcellular compartments.

### Example 4

### Low Molecular Weight (LMW) Cyclin E can Bypass Letrozole-induced G1 Arrest in Human Breast Cancer Cells and Tumors

Although treatment of postmenopausal estrogen receptor-positive breast cancer by aromatase inhibitors (AI) such as letrozole reduces risk of early metastasis, resistance develops with time. Inhibition of cyclin E/CDK2 kinase activity through increased binding of the cell cycle inhibitor p27 to the complex is a key mediator of the antiproliferative effects of letrozole. Overexpression of LMW cyclin E can bypass this process and renders letrozole ineffective in mediating growth arrest. Treatment of the cells with roscovitine overcomes the LMW cyclin E-mediated letrozole resistance. Lastly, the inventors show that breast cancer patients whose tumors overexpress LMW cyclin E are more likely to recur after AI treatment compared to those with low or no expression of LMW cyclin E. These data in this Example suggest clinical utility of CDK2 inhibitor therapy for postmenopausal women with ER-positive, LMW cyclin E expressing breast cancer.

### Materials and Methods

Chemicals. The aromatase inhibitors letrozole, exemestane and anastrozole were provided by Astra Zeneca. These drugs were dissolved in methanol and diluted in tissue culture medium. Androstenedione was obtained from Sigma Chemical Co. (St. Louis, Mo.) and the drug was dliluted in ethanol. Vehicles (methanol or ethanol) alone were used as controls.

Cell culture. MCF-7/Ac1 was cultured in improved modified Eagle's medium with 5% fetal bovine serum, 1% penicillin/streptomycin solution and 600 μg/ml G418.

Cell growth. The effects of the aromatase inhibitors on MCF7/Ac1 cells growth were examined by counting the cells at the indicated time. The cells were detached from their flask using trypsin and counted using a Coulter counter machine.

Cell cycle analysis. MCF-7/Ac1 cells were cultured in estrogen-deprived media with or without 25 nM 4-androstenedione, and treated with different concentrations of aromatase inhibitors. Untreated and drug treated cells were collected 72 hours later for flow cytometry and lysates were prepared for western blot and kinase assays.

Flow cytometry analysis. Cells were pelleted and resuspended in 1.5 ml of PBS, then fixed in 3.5 ml of 95% ethanol overnight at −20° C. After being washed, the pellets were resuspended in a solution of PBS containing 10 μg/ml propidium iodide, 20 μg/ml RNase A, 0.5% Tween 20, and 0.5% BSA and incubated at 37° C. for 30 min. The profiles of cells in the G0-G1, S, and G2-M phases of the cell cycle were analyzed at the M. D. Anderson Cancer Center Cytometry Core Facility on a FACS Caliber machine equipped with Cellquest or ModFit software.

Western blot analysis. Cell lysates were prepared and subjected to western blot analysis as described previously (Akli et al., 2004). Briefly, 50 μg of protein were subjected to electrophoresis on SDS-PAGE and transferred to Immobilon P overnight at 4° C. at 35 mV constant voltage. The blots were blocked overnight at 4° C. in BLOTTO [5% nonfat dried milk in 20 mM Tris, 137 mM NaCl, and 0.05% Tween (pH 7.6)]. After being washed, the blots were incubated in primary antibodies for 3 h. Primary antibodies used were cyclin E (HE-12; Santa Cruz Biotechnology), p21 (0P64; Oncogene Research Products, Boston, Mass.), p27 (K25020; BD Biosciences-Transduction Laboratories, Lexington, Ky.), cyclin-dependent kinase 2 (CDK2; Transduction Laboratories), and actin (Chemicon International, Inc., Temecula, Calif.). Blots were then incubated with goat anti-mouse or anti-rabbit immunoglobulin-horseradish peroxidase conjugate at a dilution of 1:5000 in BLOTTO for 1 h and finally washed and developed by using the Renaissance chemiluminescence system as directed by the manufacturer (Perkin-Elmer Life Sciences, Inc., Boston, Mass.). Western blots were quantitated by densitometric analysis using IPLab Gel software (Scientific Image Processing, Vienna, Va.). Densitometric values of actin were used to standardize for equal protein loading. These values were introduced into the software Graph-Pad Prism version 4.0 (GraphPad Software, Inc., San Diego, Calif.) for statistical analysis.

Immunoprecipitation and Immunoblotting. Two hundred fifty μg of cell extracts were used per immunoprecipitation with polyclonal antibody to cyclin E or polyclonal antibody to CDK2, coupled to protein A beads. After being washed, the immunoprecipitates were subjected to electrophoresis in 13% gels, transferred to Immobolin P, blocked, and incubated with the indicated antibodies as already described.

Protein Kinase Assays. For histone H1 kinase assays, the immunoprecipitates were incubated with kinase assay buffer containing 60 μM cold ATP, 5 μCi of [³²P]ATP, and 5 μg of histone H1 (Roche Diagnostics Corporation, Indianapolis, Ind.) in a final volume of 30 μl at 37° C. for 30 min. The products of the reaction were analyzed on 13% SDS PAGE gels, and the gels were stained, destained, dried, and exposed to X-ray film. For quantitation, the protein bands corresponding to histone H1 were excised, and the radioactivity of each band was measured by Cerenkov counting.

Study Patients. The clinical and pathologic data from 395 breast cancer patients, 390 of whom had data available regarding ER status, were previously reported by Keyomarsi et al. (2002). Another group of patients included 128 women treated for breast cancer at MDACC with aromatase inhibitors (121 with anastrozole, 4 with letrozole, 2 with exemestane and 1 with letrozole followed by exemestane) between 2001 and 2009. This group of AI-treated women were selected from patients cohort enrolled in an IRB approved protocol to study the cyclin E deregulation in breast cancer. From all the patients enrolled in this study at the time of surgery, freshly resected breast cancer tissue samples were collected and subjected to protein extraction and western blot analysis of cyclin E expression.

Statistical Analysis. Overall survival (OS) was calculated from the date of surgical excision of the primary tumor to the date of death or last follow-up. OS survival curves were computed by the Kaplan-Meier method (Kaplan and Meier, 1958). Univariate analyses of OS survival according to levels of ER and LMW cyclin E were performed with the use of a two-sided log-rank test (Cox, 1972). Results are shown as mean±SD. Differences were considered significant when the two tailed Student's t test showed differences at P<0.05.

### Results

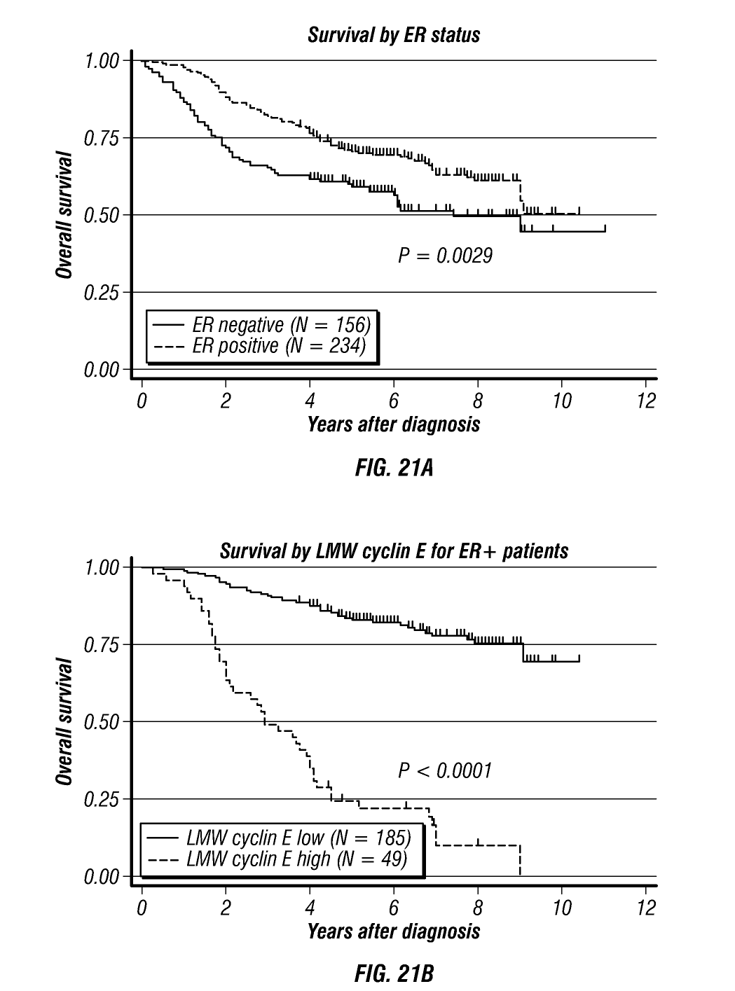

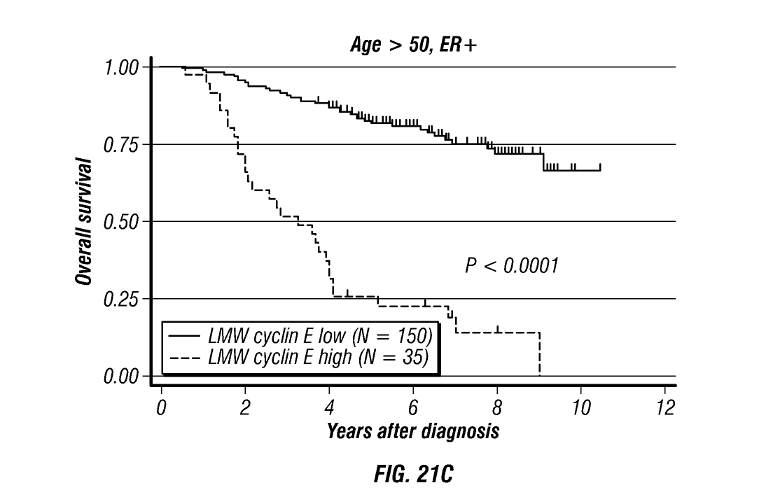

Overexpression of LMW cyclin E in postmenopausal breast cancer patients is indicative of a poor prognosis irrespective of estrogen receptor status. In a retrospective study of 395 patients, the inventors have previously reported on the strong prognostic value of cyclin E in breast cancer (Keyomarsi et al., 2002). The inventors have recently re-analyzed the data to determine the relevance of LMW cyclin E as a prognostic factor based on estrogen receptor status of the tumor (FIGS. 21A-21C). The 5-year overall survival (OS) rates were significantly higher in ER positive patients compared to ER negative patients (p=0.0029) (FIG. 21A). The inventors next stratified the 234 ER positive patients as a function of LMW cyclin E expression and found that those patients who had ER positive tumors who also had high levels of LMW cyclin E had worse outcome compared to those patients whose tumors were ER positive and had low levels of LMW cyclin E (p<0.0001) (FIG. 21B). This relationship held when only the postmenopausal patients were included in the analysis (p<0.0001), and the relationship between OS, ER and LMW cyclin E was maintained in postmenopausal patients (p<0.001) (FIG. 21C). Given this relationship between LMW cyclin E and ER status in this cohort of breast cancer patients, the inventors sought to investigate whether LMW cyclin E may effect responsiveness to hormonal therapy. The inventors specifically chose to investigate the effect on responsiveness to AIs, which currently are the standard of care for postmenopausal patients with HR positive breast cancer.

Effect of aromatase inhibitor treatment on proliferative response and cell cycle distribution of MCF-7/Ac1 cells. The inventors used MCF-7/Ac1 cells, which are MCF7 breast cancer cells that have been transfected with the gene for aromatase, the enzyme responsible for the conversion of androgens to estrogens. These cells can be stimulated to grow using the aromatizable androgen, androstenedione (AD) that is transformed into estrogen by the aromatase activity of the cells as previously reported (Zhou et al., 1990). This model system simulates the postmenopausal breast cancer patient.

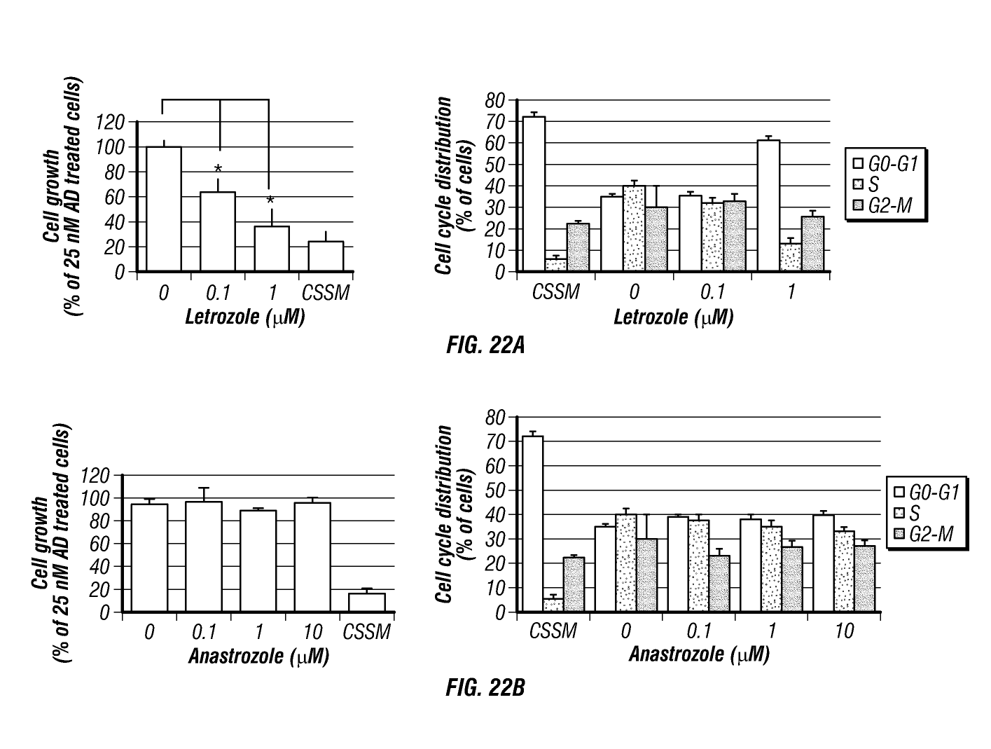

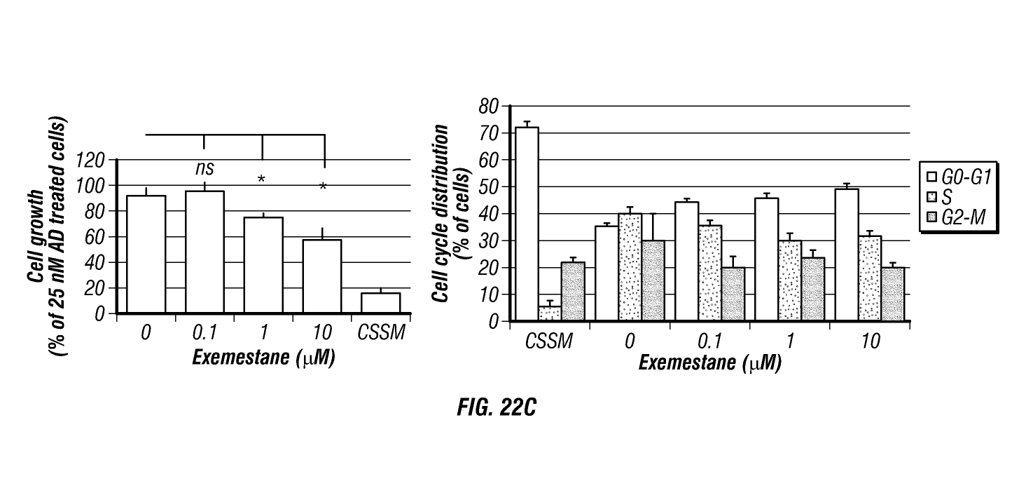

To examine the effects of the three different aromatase inhibitors on the proliferation of MCF-7/Ac1 cells, cells were cultured in estrogen deprived, charcoal stripped serum media (CSSM). The response of the cells to 25 nM of androstenedione (AD) alone or AD plus one of the 3 aromatase inhibitors letrozole (FIG. 22A), anastrozole (FIG. 22B), and exemestane (FIG. 22C) was measured after 3 days of treatment. Cells maintained in CSSM were used as controls. Compared to control cells (CSSM), 25 nM AD treatment resulted in a 3.9+/−1.3-fold increase in cell number. The AD-induced growth of MCF-7/Ac1 cells was inhibited by letrozole by 37.2+/−10.4% at 0.1 μM and 63.5+/−13.8% at 1 μM (FIG. 22A). The antiproliferative effect of letrozole is comparable to that in cells cultured in estrogen deprived media (CSSM). Anastrozole did not inhibit the AD-induced growth of MCF-7/Ac1 cells at any of the concentrations tested while exemestane partially inhibited their growth by 25+/−3% at 1 μM and 42+/−8% at 10 μM (P<0.05 vs untreated cells). These results show that MCF-7/Ac1 cells are more responsive to letrozole than to exemestane or anastrozole.

To investigate the causes of the antiproliferative effects of aromatase inhibitors, cells were stained with PI and cell cycle analysis was performed by flow cytometry (FIGS. 22A-22C, right panels). AD treatment increased the fraction of cells in S phase by 7.3-fold compared to vehicle treated cells (40.3+/−1.9% versus 5.5+/−1.7%) with a concomitant decrease in G0/G1. Among all the treatments, letrozole caused the greatest accumulation of cells (61.2+/−1.5%) in G0/G1 compared to control cells (72.1+/−1.9%) and a significant decrease in the number of cells in S phase (13.1+/−2.3%) compared to control cells (5.5+/−1.7%). Exemestane at 10 μM caused an increase in G0-G1 cells from 35.1+/−0.8% to 49.2+/−0.7% with a decrease in the number of cells in S phase from 40.3+/−1.9% to 31.7+/−2.5% while anastrozole at 10 μM had a more subtle effect. The flow cytometry data correlate with the effect observed on cell number with letrozole having the strongest antiproliferative effects on MCF-7/Ac1 cells due to disruption of cell cycle progression by causing growth arrest at the G1 phase of the cell cycle.

FIGS. 22A-22C show the effect of aromatase inhibitor treatment on proliferative response and cell cycle distribution of MCF-7/Ac1 cells. Cells were cultured in IMEM with 10% charcoal-stripped serum medium (CSSM) without phenol red and with 600 μg/ml of G418 for 4 days before plating. Cells (100,000) were seeded in 100 mm dishes and, 24 hrs later, were exposed for 3 days to the specific treatment. As shown in FIG. 22A, antiproliferative effect of increasing concentrations of letrozole in the presence of 25 nM of androstenedione (AD) on MCF-7/Ac1 cell growth (left) and cell cycle distribution (right). Cell growth is expressed as the percentage of the cells compared with the control cells (25 nM AD treated cells, 575,000 cells at day 3). Columns, mean of two to three experiments, each in triplicates; bars, S.D. *, P<0.05, when compared to cells only treated with 25 nM AD; n.s, not significant. CSSM, untreated cells cultured in charcoal-stripped serum medium without phenol red and with 600 μg/ml of G418. FIG. 22B shows the antiproliferative effect of increasing concentrations of anastrozole in the presence of 25 nM of androstenedione (AD) on MCF-7/Ac1 cell growth (left) and cell cycle distribution (right). FIG. 22C shows an antiproliferative effect of increasing concentrations of exemestane in the presence of 25 nM of androstenedione (AD) on MCF-7/Ac1 cell growth (left) and cell cycle distribution (right).

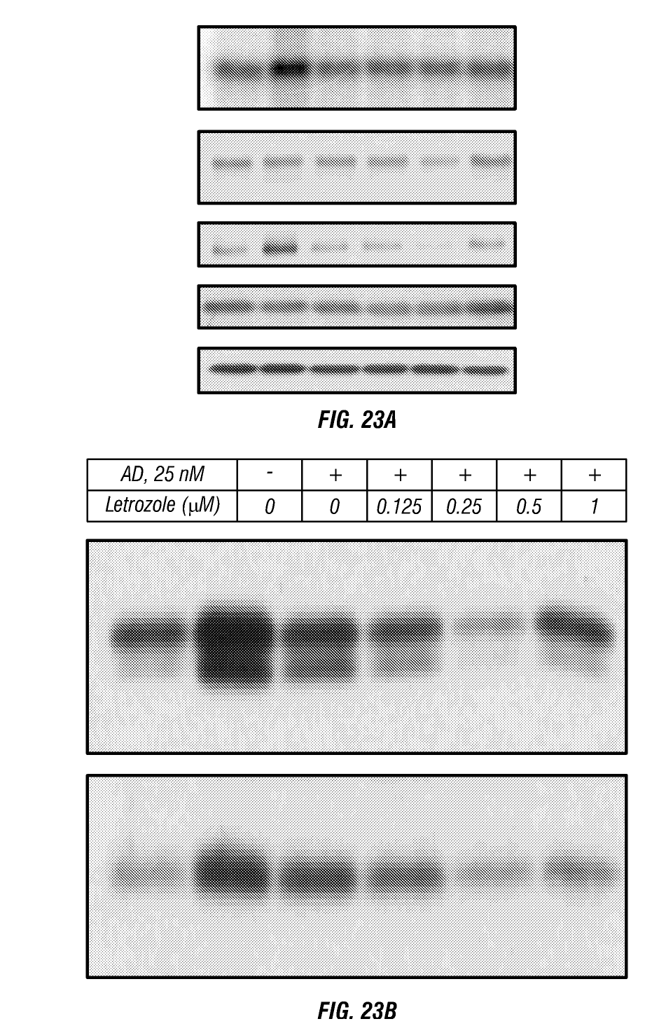

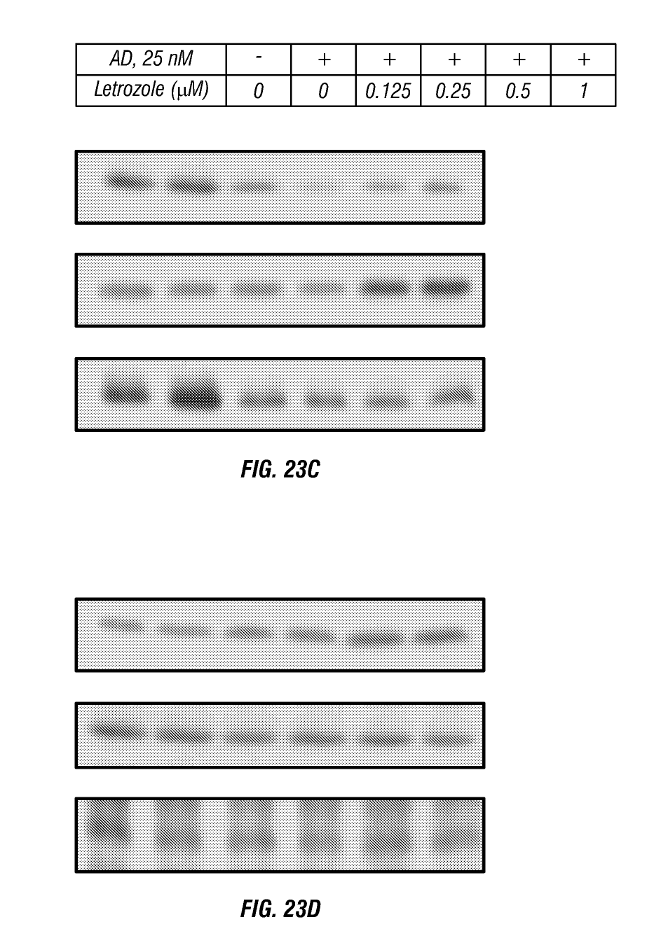

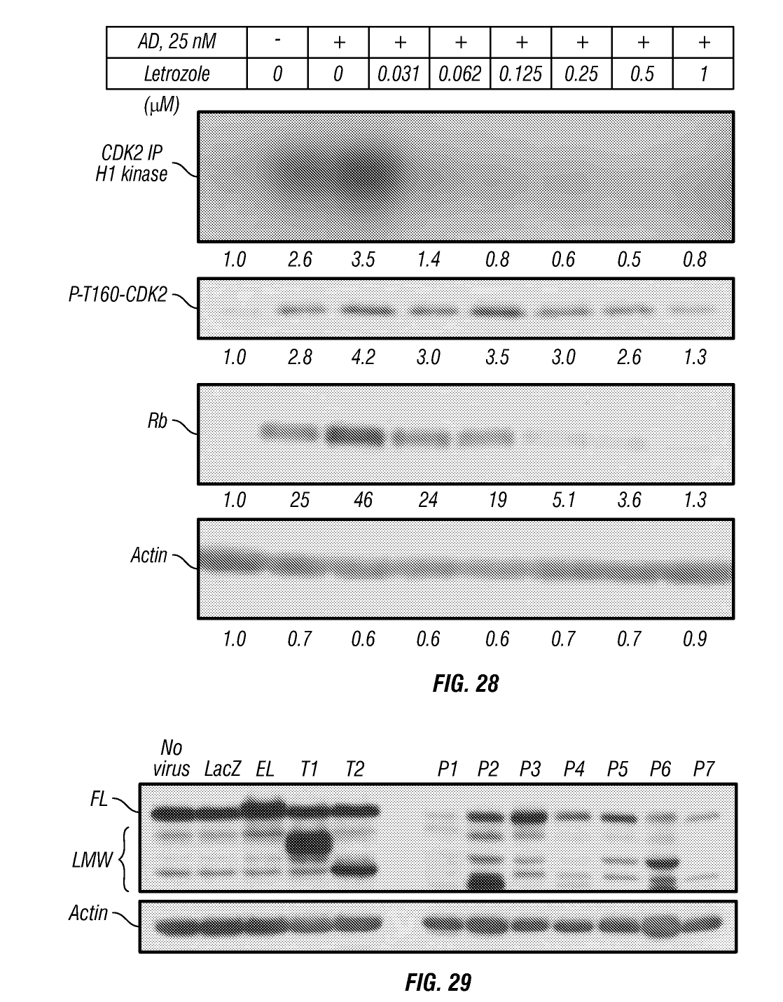

Mechanism of letrozole induced G1 arrest. After observing induction of G1 arrest by aromatase inhibitors, the inventors set out to examine the mechanism involved. Because letrozole was the most effective of the 3 inhibitors, the inventors tested this drug in subsequent experiments. The inventors investigated the effect of increasing concentrations of letrozole on the cyclin E-associated kinase activity (FIG. 23A) and on the CDK2-associated kinase activity (FIG. 28). AD treatment increased the cyclin E-associated kinase activity by 1.6-fold and the CDK2-associated kinase activity by 2.6 to 3.5-fold compared to vehicle treated cells. Letrozole blocked this increase in cyclin E- and CDK2-associated kinase activity at a concentration as low as 0.125 μM (FIG. 23A and FIG. 28). Western blot analysis showed that AD treatment increased the CDK2 protein levels by 2.3-fold when compared to vehicle treated cells while letrozole treatment blocked the increase in CDK2 protein levels in a dose dependent manner (FIG. 23B). Active CDK2 is depicted by an increase in phospho-CDK2 band shown both in western blot analysis using pan CDK2 antibody or using a phospho-specific CDK2 antibody which increased by 3.5 fold in AD treated cells. The inventors also show that increasing concentration of letrozole leads to a block of AD-induced increase in CDK2 kinase activity that parallel decreased phospho-T160-CDK2 (FIG. 28). Additionally, letrozole treatment also results in the decreased phosphorylation of the endogenous CDK2 substrate, pRb (FIG. 28). Cyclin E protein levels were not affected by AD treatment and slightly decreased at 0.5 μM letrozole. P27 protein levels remained stable and were independent of drug treatments. In order to define the molecular basis of the cyclin E and CDK2 kinase inhibition, the inventors performed immunoprecipitation with cyclin E (FIG. 23C) and CDK2 (FIG. 23D) antibodies followed by western blot for p21 and p27. While AD treatment did not affect p21 binding to cyclin E, it slightly increased the binding to CDK2 by 1.5-fold while letrozole treatment slightly decreased p21 binding to both cyclin E and CDK2. In contrast, even though p27 protein levels remained unchanged after drug treatments, p27 binding to both cyclin E and CDK2 increased in a dose-dependent manner following letrozole treatment by up to 2-fold greater than the levels in AD treated cells. These results suggest that AD induced cell proliferation and G1 exit are mediated by an increase in phospho-CDK2 activity and that letrozole inhibits these effects by preventing the AD induced increase in CDK2 activity and by inducing increased binding of p27 to cyclin E and CDK2 complexes.

FIGS. 23A-23D show in vitro antiproliferative effects of increasing concentrations of letrozole in the presence of 25 nM AD on MCF-7/Ac1 human breast cancer cells. As shown in FIG. 23A, Letrozole blocks the AD-induced increase in cyclin E-associated kinase activity in MCF-7/Ac1 cells. MCF-7/Ac1 cells were treated with the indicated concentrations of letrozole and AD for 3 days. Cyclin E kinase assays were performed by immunoprecipitating equal amounts of cell lysate (250 μg) with monoclonal antibodies to cyclin E coupled to protein A beads, using histone H1 as substrate. Letrozole blocks the AD-induced increase in CDK2 protein levels (FIG. 23B). The same cell lysates were subjected to western blot analysis (50 μg of protein) with the indicated antibodies. Letrozole induced increased binding of p27 to cyclin E complexes (FIG. 23C). The same cell lysates were first immunoprecipitated with cyclin E followed by western blot for p21 and p27. Letrozole induced increased binding of p27 to CDK2 complexes (FIG. 23D). The same cell lysates were first immunoprecipitated with CDK2 followed by western blot for p21 and p27. The levels of proteins were measured by densitometric scanning of the corresponding bands and normalized using actin values. The values indicated at the bottom were compared to the values obtained with untreated cells.

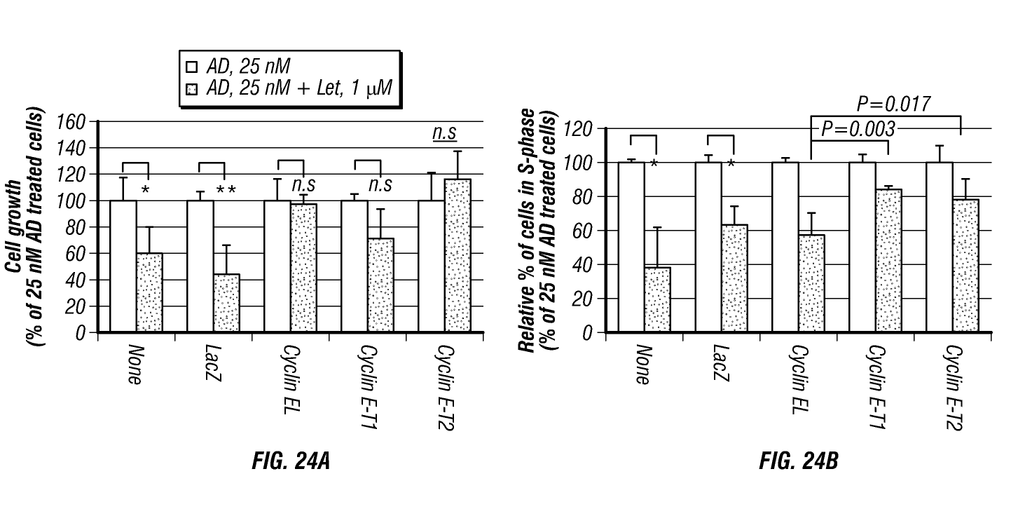

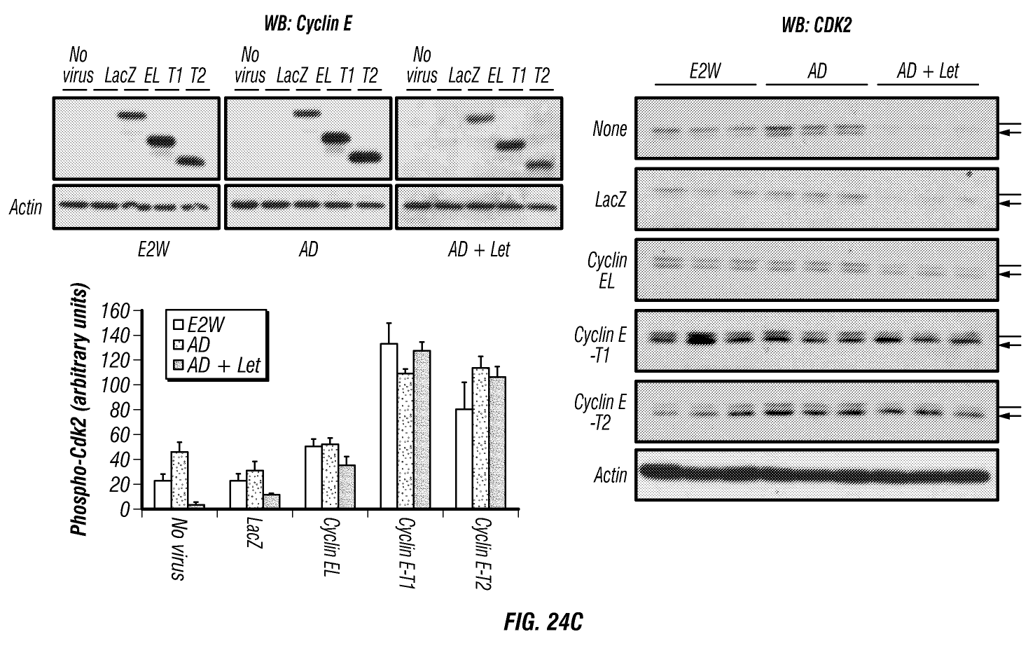

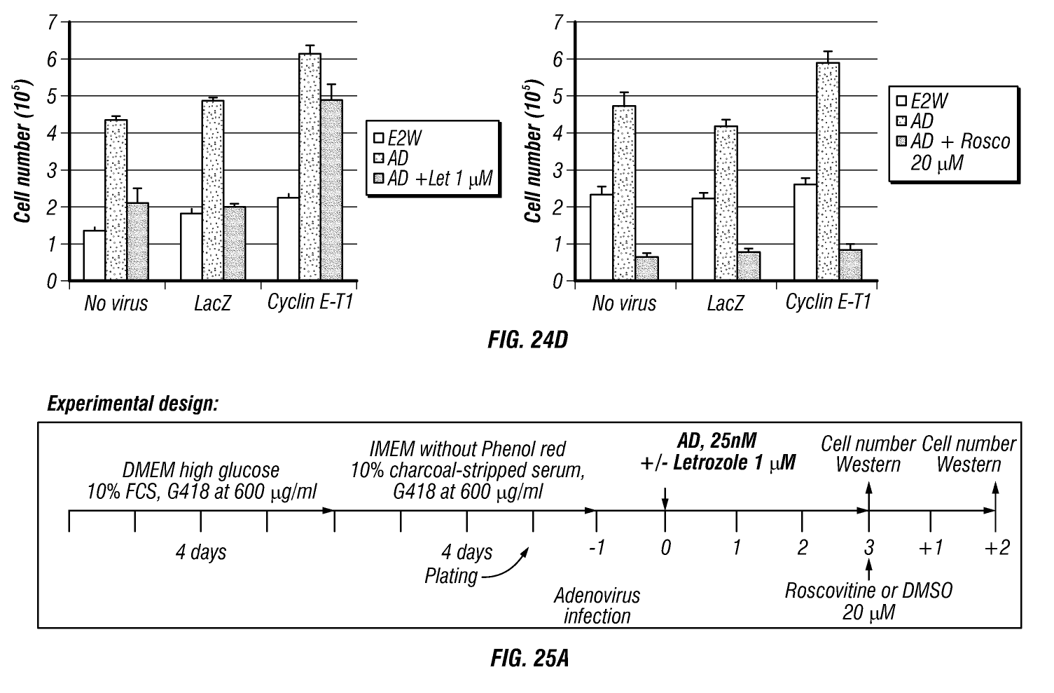

LMW cyclin E, but not full-length cyclin E, overexpressing MCF-7/Ac1 cells partially override the letrozole inhibition of AD-induced S-phase entry and AD-induced CDK2 protein levels. Since overexpression of LMW cyclin E deregulates the G1 to S transition, the inventors interrogated the role of full-length and LMW cyclin E in letrozole response. To this end, the inventors examined the sensitivity of cyclin E overexpressing MCF-7/Ac1 cells to the growth inhibitory effect of letrozole using adenoviruses to overexpress full-length and LMW cyclin E (FIGS. 24A-24D). MCF-7/Ac1 cells were cultured in charcoal stripped serum media (CSSM) for 4 days before infecting them with 4000 m.o.i. of either LacZ, full-length cyclin E (cyclin EL) or LMW cyclin E (cyclin E-T1 and cyclin E-T2). Twenty-four hours later, cells were left either untreated, treated with 25 nM AD alone, or treated with 25 nM AD plus 1 μM letrozole for an additional 3 days (FIGS. 24A-24B). Following the treatment, cells were enumerated or subjected to flow cytometry analysis. The results revealed that AD-induced growth of MCF-7/Ac1 cells was inhibited by letrozole by 40.8% (P=0.029) in uninfected cells and by 56.1% (P<0.01) in LacZ infected cells, while no significant growth inhibition was observed in cyclin EL, T1, and T2 infected cells (P>0.05) (FIG. 24A). However, flow cytometric analysis revealed that letrozole treatment caused a significant decrease in the number of cells in S phase in uninfected (62%), LacZ-infected cells (37%), and cyclin EL-infected cells (42%) while cyclin E-T1 and T2-infected cells were partially resistant to letrozole induced decrease in S phase fraction (16% and 21%, P<0.01 versus cyclin EL) (FIG. 24B). These results demonstrate that while cyclin E overexpressing cells could override the letrozole inhibition of AD-induced increase in cell number (FIG. 24A), only the LMW cyclin E overexpressing cells could override the letrozole inhibition of AD-induced S-phase entry (FIG. 24B).

FIGS. 24A-24D shows LMW but not full-length cyclin E overexpressing MCF-7/Ac1 cells could partially override the letrozole inhibition of AD-induced G1 exit and AD-induced CDK2 protein levels. As shown in FIG. 24A, cyclin E overexpression overrides the letrozole inhibition of AD-induced proliferation. MCF-7/Ac1 cells were cultured in IMEM with 10% charcoal-stripped serum medium (CSSM) without phenol red and with 600 μg/ml of G418 for 4 days before plating. Triplicate wells of 6-well plates were then infected with the indicated adenoviruses (at 4000 m.o.i.) 24 hours before drug treatment. Cells were then left untreated (E2W, estrogen withdrawal) or treated with 25 nM AD (AD) or treated with 25 nM AD and 1 μM letrozole (AD+Let) and collected 3 days later for cell number. Cell growth is expressed as the percentage of the cells compared with the control cells (25 nM AD treated cells). As shown in FIG. 24B, LMW cyclin E overexpressing MCF-7/Ac1 cells could partially override the letrozole inhibition of AD-induced G1 exit. MCF-7/Ac1 cells were treated as described in A, and collected for flow cytometry analysis. Histograms represent the S-phase fraction expressed as the percentage of the cells in S-phase compared with the control cells (25 nM AD treated cells). FIG. 24C shows that Cyclin E overexpression prevented the block by letrozole of AD-induced CDK2 protein levels. The same cell lysates as in A and B were subjected to western blot analysis (50 μg of protein) with cyclin E and CDK2 antibodies. The bar graph represents the densitometric values of the phosphorylated CDK2 bands. As shown in FIG. 24D, LMW cyclin E overexpressing MCF-7/Ac1 cells cannot bypass the block by roscovitine of AD-induced increase in cell number. Left, MCF-7/Ac1 cells were cultured in IMEM with 10% charcoal-stripped serum medium (CSSM) without phenol red and with 600 μg/ml of G418 for 4 days before plating. Cells were then infected with the indicated adenoviruses (at 4000 m.o.i.) 24 hours before drug treatment. Cells were then treated with 25 nM AD and 1 μM letrozole and collected 3 days later for cell number. Right, cells were treated as in A except that letrozole was replaced by 20 μM of roscovitine. Columns, mean of two to three experiments, each in triplicates; bars, S.D.

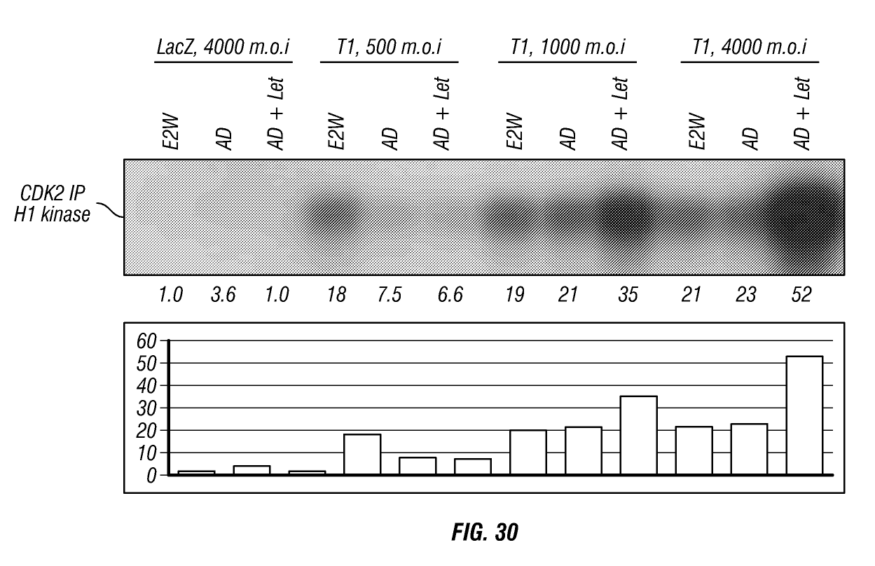

To determine if cyclin E overexpression could rescue the block by letrozole of AD-induced CDK2 protein levels, the same samples were used to determine the cyclin E and CDK2 protein levels by western blot analysis. The exogenous forms of cyclin E were expressed at 2-fold to 4-fold higher levels than endogenous cyclin E and the western blot in FIG. 24C demonstrates that the cyclin E protein levels were not affected by the drug treatments. The inventors also show that the LMW cyclin E protein levels achieved by adenoviral expression is comparable to the levels seen in human breast tumor samples (FIG. 29). AD treatment induced a 1.8-fold increase in total CDK2 protein levels in uninfected and LacZ infected cells while letrozole treatment downregulated the total CDK2 protein levels to 10% of the level in uninfected cells. Letrozole treatment of cyclin EL overexpressing cells downregulated the total CDK2 protein levels to only 50% of the level found in untreated, uninfected cells. In sharp contrast, in untreated, LMW cyclin E overexpressing cells, the CDK2 protein levels were already 3.6-fold (for cyclin E-T1) and 1.6-fold (for cyclin E-T2) higher than in untreated uninfected cells and did not drop following letrozole treatment. Densitometric scanning of the western blots revealed a 1.5 to 2-fold increase in the amount of phosphorylated, active CDK2 bands (lower band) in LMW cyclin E overexpressing cells compared to cyclin EL overexpressing cells consistent with higher CDK2 kinase activity that is resistant to letrozole inhibition (FIG. 24C, bar graph). Furthermore, increasing concentrations of the cyclin E-T1 virus increase the CDK2 kinase activity in a dose-dependent manner, 1.8- to 5-fold at 500 m.o.i, 5- to 9.7-fold at 1000 m.o.i and 5.8- to 14.4-fold at 4000 m.o.i when compared to the CDK2 kinase activity in AD-treated LacZ cells (FIG. 30). Lastly, the inventors show that 1 μM Letrozole treatment of LMW cyclin E (T1) expressing cells cannot block the CDK2 kinase activity at any of the cyclin E-T1 adenovirus m.o.i while in LacZ expressing cells letrozole completely blocks the AD-induced increase in CDK2 kinase activity (FIG. 30). These results demonstrate that cyclin E overexpression can prevent the block by letrozole of AD-induced CDK2 partially for cyclin EL and completely for cyclin E-T1 and cyclin E-T2 overexpressing cells. Cyclin E-T1 overexpression can also completely prevent the block by letrozole of AD-induced CDK2 kinase activity (FIG. 30).

LMW cyclin E overexpressing MCF-7/Ac1 cells cannot bypass the block by roscovitine of AD-induced increase in cell number. Since the inventors' results thus far demonstrated that AD and LMW cyclin E induced cell proliferation and that G1 exit is mediated by increased CDK2 protein levels and activity, the inventors questioned if a cyclin-dependent kinase inhibitor such as roscovitine could block this effect. To directly address this question, MCF-7/Ac1 cells were cultured in charcoal stripped serum media (CSSM) for 4 days before adding medium with no virus or with 4000 m.o.i. of either LacZ, or LMW cyclin E (cyclin E-T1) adenoviruses. 24 hours later, cells were left untreated, treated with 25 nM AD alone, or treated with 25 nM AD plus 1 μM letrozole for 3 days (FIG. 24D). The AD-induced growth of MCF-7/Ac1 cells was inhibited by letrozole by 51% in uninfected cells and by 58% in LacZ infected cells while the growth of cyclin E-T1 infected cells was inhibited by only 20% (FIG. 24D, left). In sharp contrast, 20 uM of Roscovitine completely inhibited the AD-induced increase in cell number in uninfected, LacZ or cyclin E-T1 infected cells (FIG. 24D, right). These results demonstrate that LMW cyclin E overexpressing MCF-7/Ac1 cells cannot bypass the block by roscovitine of AD-induced increase in cell number.

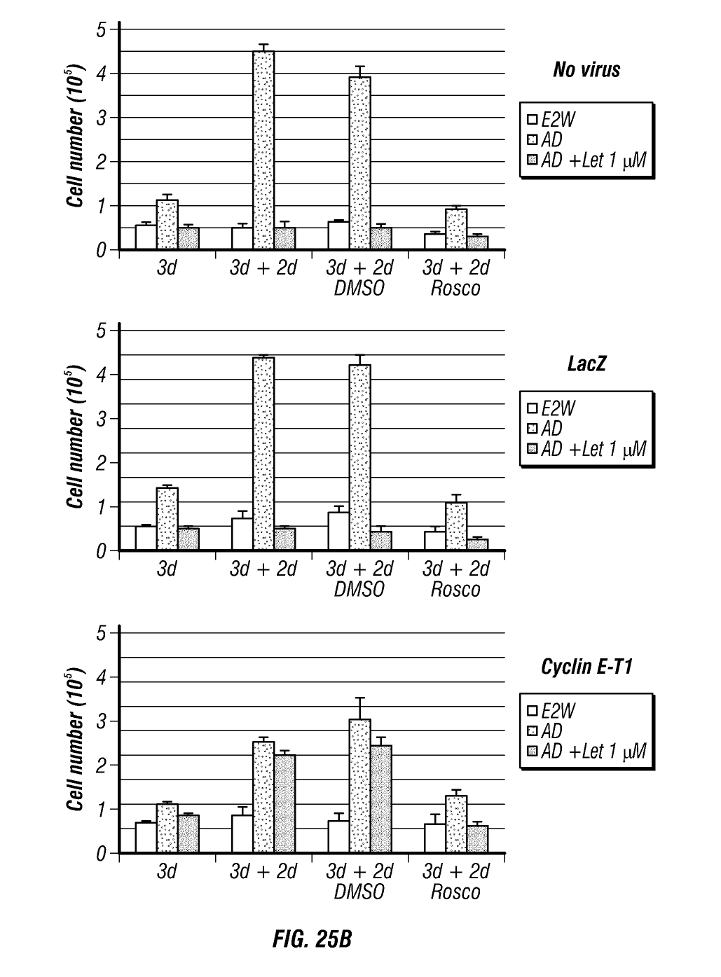

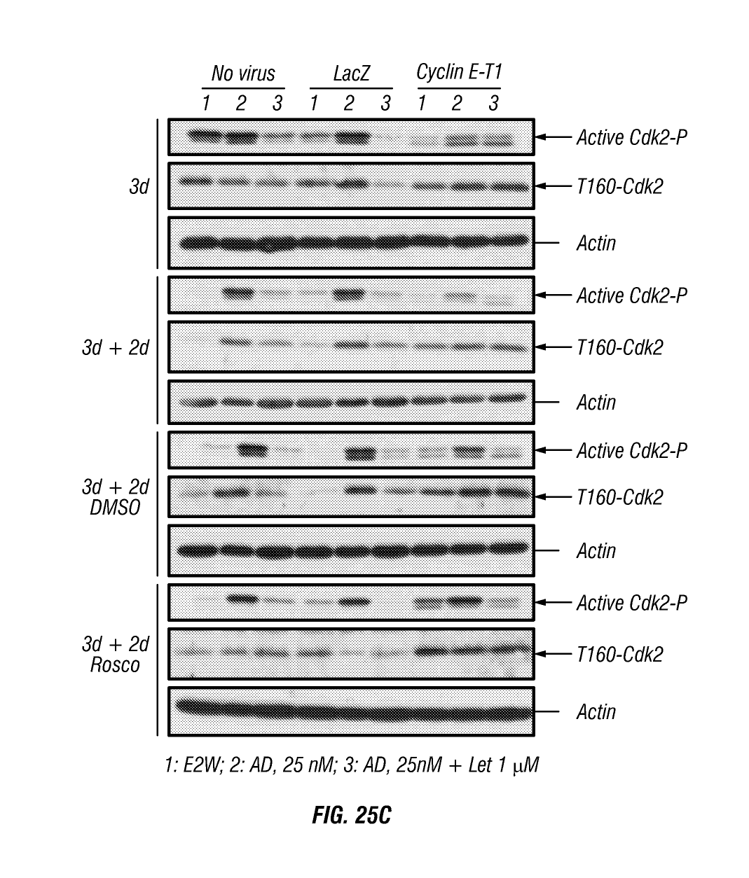

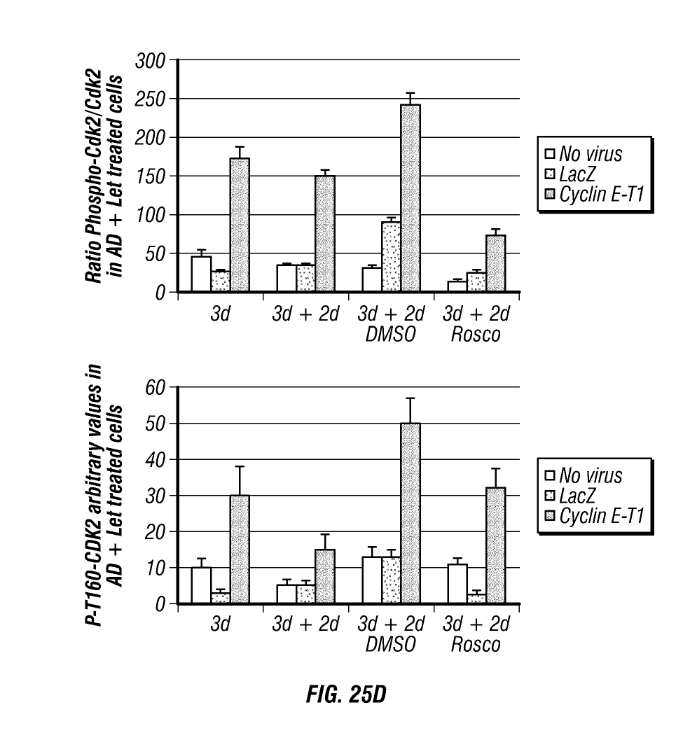

Roscovitine blocks the AD-induced increase in active (phosphorylated) CDK2 and LMW cyclin E overexpression cannot bypass this effect. The inventors next examined if roscovitine could also block the growth of letrozole-resistant LMW cyclin E overexpressing MCF-7/Ac1 cells. To this end, cyclin E-T1 (i.e. LMW) infected MCF-7/Ac1 cells were sequentially treated with AD in the presence or absence of letrozole for 3 days, followed by 20 μM of Roscovitine for an additional 2 days (FIGS. 25A-25D). Medium alone or medium plus DMSO were used as controls. A schematic of the treatment strategy is depicted in FIG. 25A. At the conclusion of the treatment, cells were enumerated and subjected to western blot analysis with CDK2 (total) and phospho-CDK2 antibodies. The results revealed that three days of letrozole treatment blocked the AD-induced increase in cell number in uninfected and LacZ infected MCF-7/Ac1 cells while LMW cyclin E overexpressing cells were resistant to letrozole inhibition (FIG. 25B). Culturing of cells for an additional two days in charcoal stripped serum medium or medium plus DMSO led to a 4- and 3.5-fold increase in AD-induced proliferation for uninfected and LacZ infected cells, respectively and a 2.3 and 2.8-fold increase in AD-induced proliferation for LMW cyclin E overexpressing cells. This AD-induced proliferation was blocked by roscovitine concomitant with the disappearance or decrease in phosphorylated, active CDK2 protein as shown by western blot analysis (FIG. 25C, lower band). In LMW cyclin E overexpressing cells, letrozole treatment did not prevent a 2.5-fold and 2.8-fold increase in AD-induced proliferation nor did it decrease the phosphorylated/unphosphorylated CDK2 ratio (134-234%) (FIG. 25D, upper). On the other hand, treatment of cells with 20 uM of roscovitine was sufficient to completely block the proliferation of letrozole resistant cells concomitant with a decrease in phosphorylated/unphosphorylated CDK2 ratio to 69% (FIG. 25D, lower). These results show that roscovitine blocks the AD-induced increase in active (phosphorylated) CDK2 and LMW cyclin E overexpression cannot bypass this effect. These results also suggest that roscovitine treatment of breast cancer cells can reverse intrinsic or acquired resistance to letrozole as a result of LMW cyclin E expression.

FIGS. 25A-25D show that Roscovitine blocks the AD-induced increase in active (phosphorylated) CDK2 and LMW cyclin E overexpression cannot bypass this effect. FIG. 25A shows a schematic representation of the experimental design. As shown in FIG. 25B, MCF-7/Ac1 cells were cultured in IMEM with 10% charcoal-stripped serum medium (CSSM) without phenol red and with 600 μg/ml of G418 for 4 days before plating at a density of 100,000 cells for a 100 mm dish. Cells were then infected with the indicated adenoviruses (at 4000 m.o.i.) 24 hours before drug treatment. Cells were then treated with 25 nM AD and 1 μM letrozole and collected 3 days later for cell number (3d) followed by 2 days in medium (CSSM) alone (3d+2d) or medium (CSSM) plus DMSO (3d+2d DMSO) or medium (CSSM) plus 20 μM of roscovitine (3d+2d Rosco). As shown in FIG. 25C, the same cells used for counting were collected and lysates were subjected to western blot analysis (25 μg of protein) with either the CDK2 (D-12) or phospho-T160-CDK2 antibody. FIG. 25D shows ratio of densitometric values of phosphorylated CDK2/total CDK2 (Upper) and densitometric values of the phospho-T160-CDK2 bands in AD+Let treated cells (Lower).

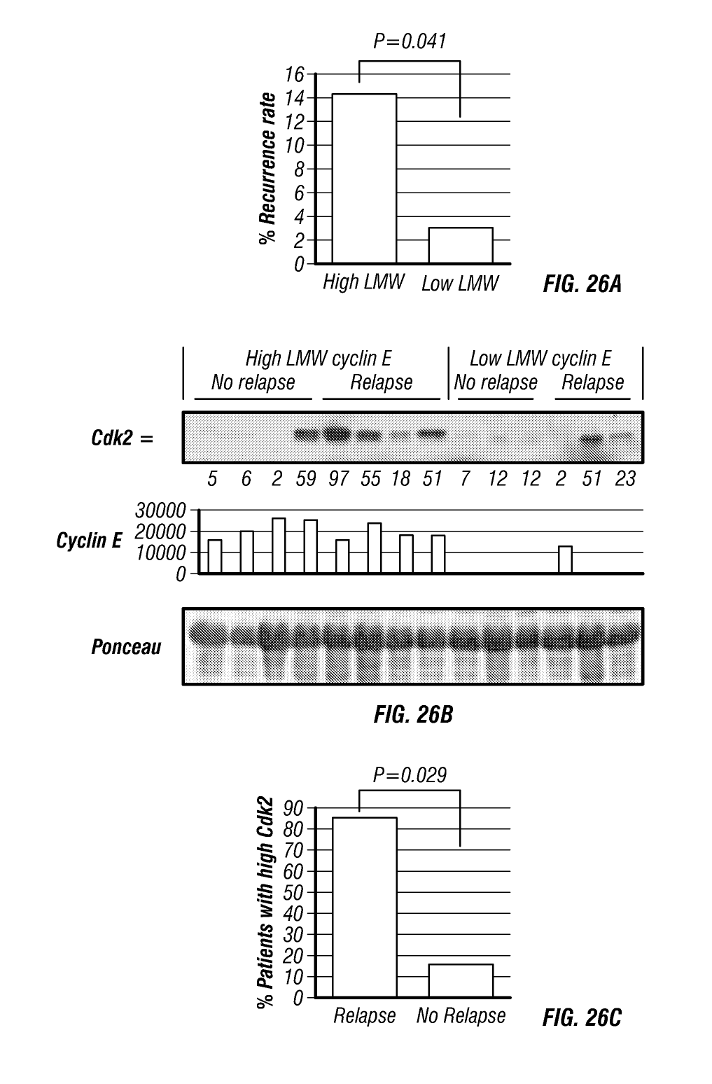

Increased risk of recurrence in AI-treated patients with high LMW levels in tumors. To determine the relationship between levels of LMW cyclin E in breast cancer tissues and resistance to AI treatment, the inventors performed an analysis of recurrence rate in 128 AI-treated breast cancer patients with high (28/128) and low (100/128) LMW levels in tumors. (FIG. 26A). The inventors found that AI-treated patients with high LMW tumors have increased frequency of recurrence (4/28, 14.3%) when compared to patients with low LMW tumors (3/100, 3.0%; Fisher's exact test P=0.041) The relative risk of disease relapse in AI-treated patients with high LMW tumors was 4.76 higher than in women with low LMW tumors. Furthermore, the inventors measured the levels of CDK2 in breast cancer tissues from patients resistant to AI based on disease relapse (n=7) as well as in tumor samples from patients being disease-free after AI treatment at the time of the last contact (n=7) (FIG. 26B). These results revealed that 6 out of 7 patients with recurrent disease had increased CDK2 protein levels compared to 1 out of 7 patients with no relapse (P=0.0291, Fisher's exact test, FIG. 26C). Among the high LMW cyclin E group, 4 out of 4 AI-resistant patients (who had relapse) had increased CDK2 protein levels compared to 1 out of 4 patients with no relapse (P=0.1429, Fisher's exact test). These results suggest that overexpression of LMW cyclin E and increased CDK2 protein levels not only can predict potential AI treatment failure, but also provide a rational basis of treatment of these patients with CDK inhibitors.

FIGS. 26A-26C show AI-treated patients with high LMW tumors have increased frequency of recurrence and increase levels of CDK2. As shown in FIG. 26A, AI-treated patients with high LMW tumors have increased frequency of recurrence (4/28, 14.3%) when compared to patients with low LMW tumors (3/100, 3.0%; Fisher's exact test P=0.041). FIG. 26B shows Western blot analysis to measure CDK2 protein levels in breast cancer tissues from patients with high LMW cyclin E levels but did not relapse (n=4), from patients with high LMW cyclin E levels who relapsed (n=4), from patients with low LMW cyclin E levels but did not relapse (n=3), and from patients with low LMW cyclin E levels who relapsed (n=3). Lysates were subjected to western blot analysis (50 μg of protein) with CDK2 (D-12, sc-6248). Total cyclin E levels were determined by western blot analysis and densitometry was used to quantitate full length and LMW forms for each sample. The densitometric values of LMW cyclin E are presented in the bar graph. Units used are arbitrary. As shown in FIG. 26C, AI-resistant tumors have increased levels of CDK2. 6 out of 7 patients with recurrent disease had increased CDK2 protein levels compared to 1 out of 7 patients with no relapse (P=0.0291, Fisher's exact test).

### Discussion

The above results show that overexpression of the LMW forms of cyclin E render letrozole therapy ineffective in breast cancer cells which express both aromatase and estrogen receptor. The mechanism of this effect is through LMW cyclin E-mediated induction of CDK2 activity. When LMW cyclin E is present, it results in higher CDK2 activity and resistance to p21 and p27 inhibition. Treatment of cells with letrozole leads to increased binding of p27 to CDK2 resulting in inactivation of CDK2. An event such as overexpression of LMW cyclin E, which can bypass this process will render letrozole ineffective in mediating a growth arrest in these cells. The inventors also show that treatment of cells with roscovitine can overcome this LMW cyclin E-mediated letrozole resistance. As such, the inventors' data provides an alternative treatment option for those postmenopausal breast cancer patients whose tumors are ER positive, but express the LMW forms of cyclin E. The inventors show that this subgroup of patients has a poor prognosis, with a median survival time of only 3.25 years. The inventors provide in vitro evidence, that if these patients were to be treated with letrozole, it is likely that they will not respond effectively to this treatment.

A major issue in the treatment of hormone receptor positive breast cancer is resistance to endocrine therapy. This resistance is intrinsic in up to 50% of patients and acquired in all patients with metastatic disease. Mechanisms of resistance to letrozole include a genetic polymorphism in the aromatase gene CYP19 (Colomer et al., 2008), high levels of ER expression driving transcription (Kuske et al., 2006) or a constitutively active estrogen receptor ER that does not require estrogen for activation (Masri et al., 2008). Cancer cells can also acquire resistance to letrozole by activation of the HER-2/MAPK pathway and in these cases trastuzumab plus letrozole has been shown to be more effective than either drug alone in letrozole-refractory tumors (Sabnis et al., 2009). Other growth factor pathways, including IGF receptor and the PI3K/AKT/mTOR pathways, have been demonstrated to play a role in resistance to endocrine agents and combination treatments targeting multiple pathways are more effective (Lisztwan et al., 2008; Beeram et al., 2007). Here the inventors show that activation of CDK2 by overexpression of the LMW forms of cyclin E is a novel mode of letrozole resistance; one that can be circumvented with CDK inhibitors.

Cyclin E protein is overexpressed and post-translationally cleaved by elastase into LMW isoforms (Porter et al., 2001). LMW cyclin E accumulation is tumor-specific and these isoforms have been found in multiple tumor types including breast, ovarian and colorectal cancers, and melanomas (Bales et al., 2005; Bedrosian et al., 2004; Corin et al., 2006; Davidson et al., 2007; Keyomarsi and Pardee, 1993). Furthermore, LMW cyclin E proteins have been shown to be strong correlative biomarkers in breast and ovarian cancers (Keyomarsi et al., 2002; Davidson et al., 2007). The LMW cyclin E isoforms have a more profound effect on cell cycle deregulation than the full-length cyclin E (EL) protein (Akli et al., 2004; Porter et al., 2001; Bedrosian et al., 2004; Corin et al., 2006; Wingate et al., 2003; Wingate et al., 2005) and transgenic mice expressing the LMW cyclin E isoforms have more mammary tumor development and metastasis than transgenic mice with the full-length cyclin E (EL) (Akli et al., 2007) Thus the LMW cyclin E isoforms appear more aggressive than EL in cell cycle abrogation and mammary tumor initiation and maintenance. Cyclin E has also been implicated in anti-estrogen resistance. A study found that the association between cyclin E and disease outcome was restricted to patients who were treated with tamoxifen in the adjuvant setting (Span et al., 2003). Another study using MCF-7 cells reported that overexpression of cyclin E could counteract tamoxifen-mediated growth arrest in human breast cancer patients (Dhillon and Mudryj, 2002). The inventors have previously shown that overexpression of LMW cyclin E in breast cancer cells is associated with resistance to fulvestrant (Akli et al., 2004). Here, the inventors describe a novel mechanism of letrozole resistance through overexpression of LMW cyclin E leading to sustained activation of CDK2. Patients with high LMW cyclin E levels and ER positive tumors would likely not respond to letrozole treatment but could benefit from a therapy targeting the cyclin E/CDK2 complexes such as Roscovitine (i.e., Seliciclib or CYC202).

Until now, the use of CDK inhibitors in human malignancies has been of limited success. This may be due to suboptimal selection of the group of patients that would benefit the most from the therapy. The inventors show in their model system that the conversion of androstenedione into estrogen by the aromatase enzyme activity strongly stimulates the growth of breast cancer cells by increasing the CDK2 kinase activity leading to increase in the S-phase fraction. The inventors' study shows that letrozole treatment blocks the AD-induced increase in S-phase fraction which would be translated to a low Ki67 labeling index in a responding tumor. The Ki67 labeling index before and after neoadjuvant endocrine therapy could identify the non-responding ER-positive, LMW cyclin E positive tumors that could benefit from a CDK2 targeted therapy. Additionally, this Example suggests that there is a need to identify the population of patients that may benefit from CDK inhibitors (i.e., overcoming the weaknesses of prior studies that were limited by poor patient selection) and that these data suggest that tumors from patients with ER positive disease should be assessed for expression of LMW cyclin E in an effort to predict who may respond to letrozole and who could also benefit from CDK2 targeted therapy.

### Example 5

### Synergistic Effect of Trastuzumab Therapy and Roscovitine

Having demonstrated decreased proliferation after trastuzumab treatment, the inventors investigated the effect of further overexpression of LMW cyclin E on this therapy. HER2-overexpressing SKBr3 cells were infected with a LMW cyclin E adenovirus resulting in overexpression of the T2 LMW isoforms. Cells were treated with trastuzumab and proliferation assessed using an MTT assay. Further expression of LMW cyclin E by infection with the adenovirus resulted in inhibition of the anti-proliferative effect seen after trastuzumab treatment, however this did not reach statistical significance. The inventors attribute this in part to the fact that the SKBr3 cells already express the LMW forms and the additional increase in expression achieved by infection with the adenovirus does not further rescue the cells from the treatment effect of trastuzumab.

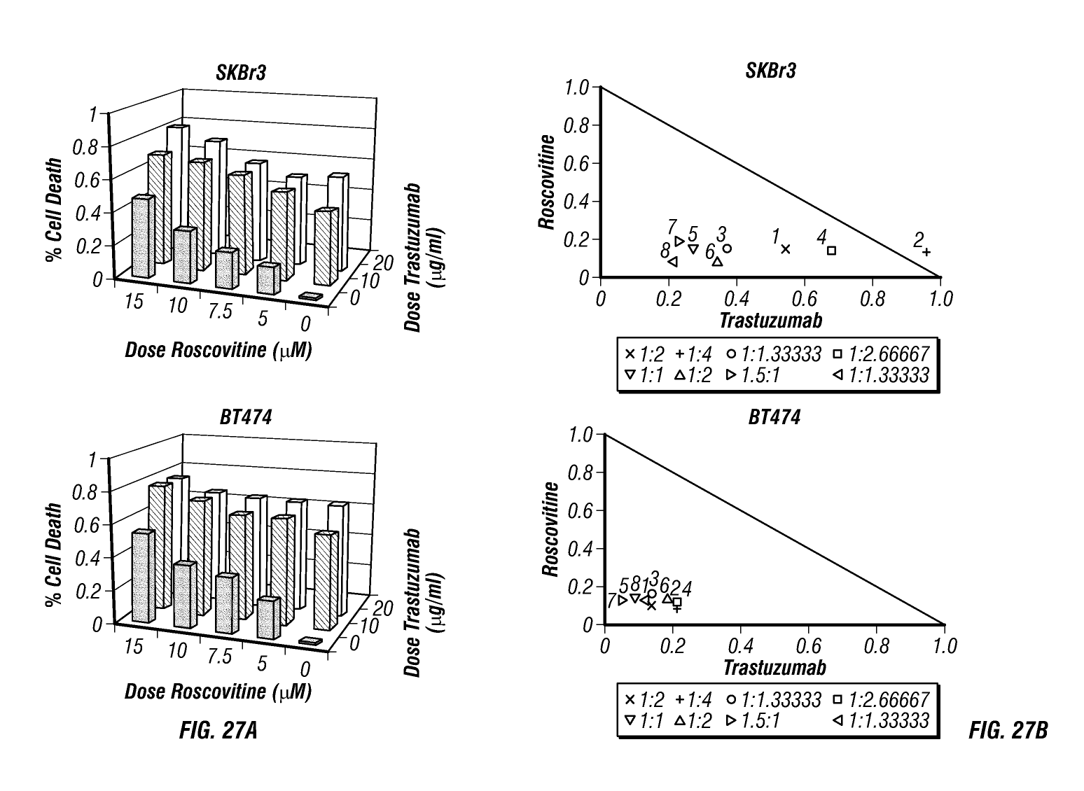

These data suggested potential utility in targeting cyclin E and HER2. The inventors therefore investigated the effects of combining trastuzumab with roscovitine, an olomucine-related purine that inhibits CDK2/cyclin E-associated kinase activity. Highthroughput clonogenic assays were used to compare cytotoxic effects of trastuzumab alone, roscovitine alone, or the combination in SKBr3 and BT474 cells. When given individually, both agents showed a dose-dependent reduction in cell viability (FIG. 27A). With the combination, there was significantly decreased cell viability compared with either agent alone. The combination index showed synergistic cytotoxicity between the two agents (FIG. 27B).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Akli and Keyomarsi, Cancer Biol. Ther., 2(4 Suppl 1):S38-47, 2003.
Akli et al., Cancer Res., 64:3198-208, 2004.
Akli et al., Cancer Res., 67:7212-22, 2007.
Bacus et al., Cancer Res., 52(9):2580-9, 1992.
Bales et al., Cancer Res., 65:692-7, 2005.
Band and Sager, Proc. Natl. Acad. Sci. USA, 86:1249-53, 1989.
Band et al., Proc. Natl. Acad. Sci. USA, 87:463-467, 1990.
Bedrosian et al., Oncogene, 23:2648-57, 2004.
Beeram et al., Ann. Oncol., 18:1323-8, 2007.
Ben-Bassat et al., Cancer Res., 57(17):3741-50, 1997.
Buckley et al., Oncogene, 8(8):2127-33, 1993.
Cam et al., Dig. Dis. Sci., 46:2187-98, 2001.
Chen et al., Nature, 374(6520):386-8, 1995.
Chen et al., Oncogene, 18:5691-702, 1999.
Coates et al., J. Clin. Oncol., 25:486-92, 2007.
Cobleigh et al., J. Clin. Oncol., 17(9):2639-48, 1999.
Colomer et al., Clin. Cancer Res., 14:811-6, 2008.
Coombes et al., Lancet, 369:559-70, 2007.
Corin et al., Cancer Biol. Ther., 5:198-203, 2006.
Cox, J. R. Stat. Soc., 34:187-220, 1972.
Davidson et al., Cancer, 110:1264-71, 2007.
Ding et al., Proc. Natl. Acad. Sci. USA, 103:15014-9, 2006.
Dong et al., Clin. Cancer Res., 6:4253-8, 2000.
Donnellan and Chetty, Faseb J., 13:773-80, 1999.
Dou et al., Nat. Med., 2:254, 1996.
Dulic et al., Proc. Natl. Acad. Sci. USA, 90(23):11034-8, 1993.
Ellis and Ma, Breast Cancer Res. Treat., 105(Suppl 1):33-43, 2007.
Forbes et al., Lancet Oncol., 9:45-53, 2008.
Furstenthal et al., J. Cell Biol., 152:1267-78 2001.
Geisen and Moroy, J. Biol. Chem., 277:39909-18, 2002.
Generali et al., J. Clin. Oncol., 27:227-34, 2009.
Gray-Bablin Ding et al., Proc. Natl. Acad. Sci. USA, 93:15215-20, 1996.
Gusterson et al., J. Clin. Oncol., 10(7):1049-56, 1992.
Hanahan and Weinberg, Cell, 100: 57-70, 2000.
Harwell et al., Cancer Res., 60(2):481-4, 2000.
Harwell et al., Cancer Res., 60:481-9, 2000.
Honda et al., Embo J., 24:452-63, 2005.
Jackman et al., Mol. Biol. Cell, 13:1030-45, 2002.
Johnson and Walker, Annu. Rev. Pharmacol. Toxicol., 39:295-312, 1999.
Jong et al., Eur. J. Surg. Oncol., 25:157-63, 1999.
Kaplan and Meier, J. Am. Stat. Assoc., 53:457-81, 1958.
Kelly et al., Proc. Natl. Acad. Sci. USA, 95:2535-40, 1998.
Keyomarsi and Herliczek, Prog. Cell Cycle Res., 3:171-91, 1997.
Keyomarsi and Pardee, Proc. Natl. Acad. Sci. USA, 90(3):1112-6, 1993.
Keyomarsi et al., Cancer Res., 54(2):380-5, 1994.
Keyomarsi et al., N. Engl. J. Med., 347(20):1566-75, 2002.
Kiyokawa et al., Cell, 85:721-32, 1996.
Koepp et al., Science, 294:173-7, 2001.
Koutsami et al., J. Pathol., 209:512-21, 2006.
Kuske et al., Endocr. Relat., Cancer, 13:1121-33, 2006.
Le et al., Cell Cycle, 5(15):1654-61, 2006.
Lee et al., Mol. Cell. Biol., 20(2):672-83, 2000.
Lew et al., Cell, 66:1197-206, 1991.
Lindahl et al., Carcinogenesis, 25:375-80, 2004.
Lisztwan et al., Breast Cancer Res., 10:R56, 2008.
Masri et al., Cancer Res., 68:4910-8, 2008.
Matsumoto and Maller, Science, 306:885-8, 2004.
McCann et al., Cancer Res., 51(12):3296-303, 1991.
McGahren-Murray et al., Cancer Res., 66:9744-53, 2006.
Melck et al., Ann. Surg. Oncol., 14:3403-11, 2007.
Milne et al., J. Clin. Pathol., 61:311-6, 2008.
Moore et al., Mol. Biol. Cell, 13:4388-400, 2002.
Moroy and Geisen, Int. J. Biochem. Cell Biol., 36:1424-39, 2004.
Nagai et al., Nature Biotechnology, 20:87-90, 2002.
Nishimura et al., Genes Cells, 10:75-86, 2005.
Normanno et al., Endocr. Relat. Cancer, 12:721-47, 2005.
Ohtsubo and Roberts, Science, 259(5103):1908-12, 1993.
Ohtsubo et al., Mol. Cell. Biol., 15(5):2612-24, 1995.
Peng et al., Cancer Res., 56(16):3666-9, 1996.
Porter et al., Mol. Cell. Biol., 21(18):6254-69, 2001.
Potemski et al., J. Exp. Clin. Cancer Res., 25:59-64, 2006.
Rao et al., Oncogene, 17(18):2393-402, 1998.
Remington's Pharmaceutical Sciences, 2005.
Remy and Michnick, Methods, 32:381-8, 2004.
Sabnis et al., Cancer Res., 69(4):1416-28, 2009.
Salani et al., Clin. Cancer Res., 13:5805-9, 2007.
Salon et al., Oncogene, 26:6927-36, 2007.
Scuderi et al., Blood, 87:3360-7, 1996.
Singer et al., Genes Dev., 13:2375-87, 1999.
Slamon et al., Science, 235(4785):177-82, 1987.
Slamon et al., Science, 244(4905):707-12, 1989.
So et al., J. Surg. Res., 94:56-60, 2000.
Span et al., Oncogene, 22:4898-904, 2003.
Spruck et al., Nature, 401(6750):297-300, 1999.
Sui et al., Gynecol. Oncol., 83:56-63, 2001.
Tannoch et al., Adv. Exp. Med. Biol., 465:127-40, 2000.
Thurlimann et al., N. Engl. J. Med., 353:2747-57, 2005.
Timms et al., Oncogene, 21(43):6573-86, 2002.
van Drogen et al., Mol. Cell, 23:37-48, 2006.
Viale et al., J. Clin. Oncol., 26:5569-75, 2008.
Vogel et al., J. Clin. Oncol., 20(3):719-26, 2002.
Watai et al., Genes Cells, 12:1163-78, 2007.
Welcker et al., Proc. Natl. Acad. Sci. USA, 101:9085-90, 2004.
Welker et al., Mol. Cell, 12:381-92, 2003.
Willmarth et al., Breast Cancer Res., 6:R531-9, 2004.
Wingate et al., Cell Cycle, 2:461-6, 2003.
Wingate et al., J. Biol. Chem., 280(15):15148-57, 2005.
Wright et al., Cancer Res., 49(8):2087-90, 1989.
Yang et al., J. Biol. Chem., 275(32):24735-9, 2000.
Yarden and Sliwkowski, Nat. Rev. Mol. Cell. Biol., 2(2):127-37, 2001.
Yasui et al., Virchows Arch., 429:13-9, 1996.
Ye et al., Mol. Cell. Biol., 23:8586-600, 2003.
Yu and Hung, Oncogene, 19(53):6115-21, 2000.
Zhao et al., Genes Dev., 14:2283-97, 2000.
Zhou et al., Cancer Res., 50:6949-54, 1990.
Zhou et al., Nat. Cell Biol., 3(3):245-52, 2001.

1. An ex vivo method of assessing a cancer patient's responsiveness to an anti-Her2 therapy or an anti-aromatase therapy, the method comprising subjecting cells of the patient's cancer, or cellular material of such cells, to an assay that determines the level of LMW-E wherein:
a) an overexpression of LMW-E in such cells relative to a normal control is an indication that the patient will more likely exhibit a favorable response to an anti-Her2 therapy;
b) an overexpression of LMW-E in such cells relative to a normal control is an indication that the patient is less likely to exhibit a favorable response to an anti-aromatase therapy; and/or
c) an expression level of LMW-E in such cells that is not overexpressed relative to a normal control is an indication that the patient is more likely to exhibit a favorable response to an anti-aromatase therapy.

2. The method of claim 1, further defined as a method of assessing a cancer patient's responsiveness to anti-Her2 therapy, wherein an overexpression of LMW-E in such cells relative to a normal control is an indication that the patient will more likely exhibit a favorable response to anti-Her2 therapy.

3. The method of claim 1, wherein:
a) an overexpression of LMW-E in such cells relative to a normal control is an indication that the patient is less likely to exhibit a favorable response to an anti-aromatase therapy; and/or
b) an expression level of LMW-E in such cells that is not overexpressed relative to a normal control is an indication that the patient is more likely to exhibit a favorable response to an anti-aromatase therapy.

4. The method of claim 1, further comprising providing a report indicative of the results of such an assessment in a tangible medium.

5. The method of claim 4, wherein the report is generated by a densitometer in a computer-readable format.

6. The method of claim 1, wherein said method comprises using an immunologic reaction device.

7. The method of claim 6, wherein the device detects immunological reactions through a photo-detection reader.

8. The method of claim 2, wherein the cancer patient has a Her-2 positive cancer.

9. The method of claim 1, wherein the normal control level of LMW-E expression is the level of LMW-E in normal cells.

10. (canceled)

11. The method of claim 1, wherein the assay comprises using an antibody that binds LMW-E, and determining the level of antibody binding.

12. The method of claim 11, wherein the assay comprises using an antibody that binds both cyclin E and LMW-E.

13. The method of claim 12, wherein the assay further comprises:
a) separating cyclin E and LMW-E in the cells or cellular material, into essentially distinct preparations stratified by one or more of subcellular localization, molecular mass, net molecular charge, and topology; and
b) measuring levels of antibody binding to LMW-E in the cells or the cellular material as compared to the normal control.

14. The method of claim 12, wherein the method comprises determining LMW-E expression by contacting the cells or cellular material thereof with a first antibody that binds cyclin E and does not bind LMW-E, and a second antibody that binds cyclin E and LMW-E.

15. The method of claim 14, wherein the first antibody carries a first label and the second antibody carries a second label.

16. The method of claim 15, wherein the assay is carried out in a device comprising a plurality of reaction chambers, and the report is generated by a device that permits assessment of such an assay.

17. The method of claim 2, wherein if the patient has the LMW-E overexpression, then the method further comprises treating the patient with an anti-Her2 therapy.

18. The method of claim 17, wherein if the patient has the LMW-E overexpression, then the method further comprises treating an anti-Her2 therapy in combination with an anti-CDK2 therapy.

19. The method of claim 3, wherein if the patient has the LMW-E overexpression, then the method further comprises treating the patient with an anti-CDK2 therapy.

20. The method of claim 3, wherein if the patient does not have the LMW-E overexpression, then the method further comprises treating the patient with an anti-aromatase therapy.

21. The method of claim 3, wherein the anti-aromatase therapy is letrozole, exemestane, or anastrozole.

22. A method of treating a cancer patient having Her2-expressing cells, wherein said patient has been determined to have a higher expression of LMW-E in Her2-expressing cells of the patient's cancer relative to a normal control, the method comprising treating the patient with an anti-Her2 therapy or an anti-CDK2 therapy.

23-27. (canceled)

28. A method for treating a cancer patient whose cancer cells do not have LMW-E overexpression relative to a normal control, comprising treating such a patient with an anti-aromatase therapy.

29. (canceled)

30. A method for treating a cancer patient whose cancer cells have a higher LMW-E expression relative to a normal control or who has an unfavorable response to an anti-aromatase therapy, comprising treating the patient with an anti-CDK2 therapy.

31-33. (canceled)

34. An ex vivo method of assessing the aggressiveness and metastatic potential of a tumor, the method comprising determining in cells of the tumor LMW-E cytoplasmic expression, and wherein higher LMW-E cytoplasmic accumulation in the cells of the tumor relative to a normal control is an indication that the tumor is aggressive and potentially metastatic.

35-57. (canceled)
